(19) United States
(12) Patent Application Publication
(10) Pub. No.: US 2025/0331853 A1
(43) Pub. Date: Oct. 30, 2025

(54) SURGICAL STAPLER WITH FIRING LOCKOUT FEATURE COUPLED TO END EFFECTOR KNIFE

(71) Applicant: Cilag GmbH International, Zug (CH)

(72) Inventors: Joseph T. Mozloom, JR., Cincinnati, OH (US); Mark D. Overmyer, Cincinnati, OH (US); Adam D. Hensel, Cincinnati, OH (US); Grant Nair, Cincinnati, OH (US); Seth D. Holdmeyer, Sharonville, OH (US)

(21) Appl. No.: 18/650,562

(22) Filed: Apr. 30, 2024

(51) Int. Cl. A61B 17/072; A61B 17/00; A61B 17/068

(52) U.S. Cl. CPC A61B 17/07207; A61B 17/0684; A61B 2017/00353; A61B 2017/00477; A61B 2017/07257; A61B 2017/07271; A61B 2017/07278; A61B 2017/07285

(57) ABSTRACT

An apparatus (1000) includes an end effector (1200, 2200, 2300, 3200, 4200, 5200), a first flexible coil (508, 1508), a second flexible coil (510, 1510), and a pivoting lock (1270, 2270, 2370, 3270, 4270, 5270). The end effector includes a first jaw (1202, 2202, 2302, 3202, 4202, 5202), a second jaw (1202), and a knife (1206, 2206, 2306, 3206, 4206, 5206). The first and second flexible coils are operatively attached to an upper portion (1238, 2238, 2338, 3238, 4238, 5238) and a lower portion (1246, 2246, 2346, 3246, 4246, 5246) of the knife, respectively. The pivoting lock is pivotable from an unlocked configuration to a locked-out configuration about a pivot axis located vertically between the first and second flexible coils to inhibit distal actuation of the knife. The knife can move the pivoting lock relative to the first and second jaws along the firing stroke.

### BACKGROUND

In some settings, endoscopic surgical instruments may be preferred over traditional open surgical devices to minimize the size of the surgical incision as well as post-operative recovery time and complications. Consequently, some endoscopic surgical instruments may be suitable for placement of a distal end effector at a desired surgical site through the cannula of a trocar. These distal end effectors may engage tissue in a number of ways to achieve a diagnostic or therapeutic effect (e.g., endocutter, grasper, cutter, stapler, clip applier, access device, drug/gene therapy delivery device, and energy delivery device using ultrasound, RF, laser, etc.). Endoscopic surgical instruments may include a shaft that extends proximally from the end effector to a handle portion that is manipulated by the clinician, or alternatively to a robot. Such a shaft may enable insertion to a desired depth and rotation about the longitudinal axis of the shaft, thereby facilitating positioning of the end effector within the patient. Positioning of an end effector may be further facilitated through inclusion of one or more articulation joints or features, enabling the end effector to be selectively articulated or otherwise deflected relative to the longitudinal axis of the shaft.

Examples of endoscopic surgical instruments include surgical staplers. Some such staplers are operable to clamp down on layers of tissue, cut through the clamped layers of tissue, and drive staples through the layers of tissue to substantially seal the severed layers of tissue together near the severed ends of the tissue layers. Such endoscopic surgical staplers may also be used in open procedures and/or other non-endoscopic procedures. By way of example only, a surgical stapler may be inserted through a thoracotomy and thereby between a patient's ribs to reach one or more organs in a thoracic surgical procedure that does not use a trocar as a conduit for the stapler. Such procedures may include the use of the stapler to sever and close a vessel leading to an organ, such as a lung. For instance, the vessels leading to an organ may be severed and closed by a stapler before removal of the organ from the thoracic cavity. Of course, surgical staplers may be used in various other settings and procedures.

The surgical stapling features of the present disclosure seek to inhibit firing of a surgical stapler end effector when the end effector is loaded with a spent staple cartridge that has already been fired, and/or when a staple cartridge is entirely absent from the end effector. Specifically, such features of the present disclosure place the end effector in a lockout state that inhibits firing in either of such scenario. While various kinds of surgical staplers and associated components have been made and used, it is believed that no one prior to the inventor(s) has made or used the invention described in the appended claims.

### BRIEF DESCRIPTION OF THE DRAWINGS

The accompanying drawings, which are incorporated in and constitute a part of this specification, illustrate examples of the invention, and, together with the general description of the invention given above, and the detailed description of the examples given below, serve to explain the principles of the present invention.

FIG. 1 is a perspective view of an illustrative surgical instrument having a housing, a shaft assembly, an articulation joint, and an end effector;

FIG. 2 is a partial perspective view of the surgical instrument of FIG. 1, with select components omitted from view to reveal portions of a cable articulation subsystem, a knife firing subsystem, and a roll subsystem of the surgical instrument;

FIG. 3 is an enlarged perspective view of the end effector and the articulation joint of the surgical instrument of FIG. 1;

FIG. 4 is an exploded view of a distal end portion of the surgical instrument of FIG. 1;

FIG. 5 is an enlarged perspective view of a knife of the end effector of the surgical instrument of FIG. 1;

FIG. 6 is an end view of the end effector of FIG. 3;

FIG. 7 is an enlarged perspective view of the end effector and the articulation joint of FIG. 3, with an anvil of the end effector omitted;

FIG. 8A is a side cross-sectional view of a distal end portion of the surgical instrument of FIG. 1, depicting the anvil in an open position;

FIG. 8B is a side cross-sectional view of the distal end portion of the surgical instrument of FIG. 1, depicting the anvil in a grasping position with the knife partially advanced;

FIG. 8C is a side cross-sectional view of the distal end portion of the surgical instrument of FIG. 1, depicting the anvil in a clamping position with the knife partially advanced;

FIG. 8D is a side cross-sectional view of the distal end portion of the surgical instrument of FIG. 1, depicting the anvil in the clamping position with the knife fully advanced;

FIG. 9A is an enlarged side cross-sectional view of a proximal end portion of the end effector of the surgical instrument of FIG. 1, depicting the anvil in the open position;

FIG. 9B is an enlarged side cross-sectional view of the proximal end portion of the end effector of the surgical instrument of FIG. 1, depicting the anvil in a grasping position with the knife partially advanced;

FIG. 9C is an enlarged side cross-sectional view of the proximal end portion of the end effector of the surgical instrument of FIG. 1, depicting the anvil in a clamping position with the knife partially advanced;

FIG. 9D is an enlarged side cross-sectional view of the proximal end portion of the end effector of the surgical instrument of FIG. 1, depicting the anvil in the clamping position with the knife fully advanced;

FIG. 10 is an exploded perspective view of the articulation joint of the surgical instrument of FIG. 1;

FIG. 11 is an end view of the articulation joint of FIG. 10;

FIG. 12 is a cross-sectional view of a portion of the articulation joint of FIG. 10, taken along line 12-12 in FIG. 11;

FIG. 13 is a cross-sectional view of a portion of the articulation joint of FIG. 10, taken along line 13-13 in FIG. 11;

FIG. 14 is a perspective view of the distal end of the surgical instrument of FIG. 1, depicting the end effector articulated vertically and laterally with the anvil open;

FIG. 15 is a side view of the distal end of the surgical instrument of FIG. 1, depicting the end effector articulated vertically with the anvil closed;

FIG. 16 is a top view of the distal end of the surgical instrument of FIG. 1, depicting the end effector articulated laterally with the anvil closed;

FIG. 17 is an exploded perspective view of a portion of the surgical instrument of FIG. 1, depicting portions of the cable articulation subsystem, the knife firing subsystem, and the roll subsystem;

FIG. 18 is a top view of a proximal end of the surgical instrument of FIG. 1, depicting portions of the cable articulation subsystem, the knife firing subsystem, and the roll subsystem;

FIG. 19 is a perspective view of a shaft assembly, a differential, and a firing rod of the surgical instrument of FIG. 1;

FIG. 20 is an exploded view of an alternative end effector having a knife lockout assembly;

FIG. 21 is a perspective view of a knife of the end effector of FIG. 20;

FIG. 22 is a perspective view of a pivoting lockout body of the knife lockout assembly of FIG. 20;

FIG. 23A is a cross-sectional view of the end effector for FIG. 20 with an unfired staple cartridge loaded into a cartridge jaw of the end effector, where the knife of FIG. 21 is in a proximal position;

FIG. 23B is a cross-sectional view of the end effector of FIG. 20 with the staple cartridge of FIG. 23A loaded into the cartridge jaw of FIG. 23A, where the knife of FIG. 21 is actuated distally from the proximal position such that the pivoting lockout body of FIG. 22 engages a cartridge sled of the staple cartridge;

FIG. 23C is a cross-sectional view of the end effector of FIG. 20 with the staple cartridge of FIG. 23A loaded into the cartridge jaw of FIG. 23A, where the knife of FIG. 21 is actuated distally while the pivoting lockout body of FIG. 22 remains engaged with the cartridge sled of FIG. 23B;

FIG. 24A is a cross-sectional view of the end effector of FIG. 20 without the staple cartridge of FIG. 23A loaded into the cartridge jaw of FIG. 23A, where the knife of FIG. 21 is in the proximal position;

FIG. 24B is a cross-sectional view of the end effector of FIG. 20 without the staple cartridge off FIG. 23A loaded into the cartridge jaw of FIG. 23A, where the knife of FIG. 21 is actuated distally such that the pivoting lockout body contacts a recessed pocket defined by the cartridge jaw, thereby inhibiting further distal actuation of the knife;

FIG. 25 is a perspective view of another alternative knife;

FIG. 26 is a perspective view of another pivoting lockout body that may readily couple with the knife of FIG. 25;

FIG. 27A is a cross-sectional view of another alternative end effector with an unfired staple cartridge loaded into a cartridge jaw of the end effector, where the end effector includes the knife of FIG. 25 and an alternative lockout assembly including the pivoting lockout body of FIG. 26, where the knife is in a proximal position;

FIG. 27B is a cross-sectional view of the end effector of FIG. 27A with the staple cartridge of FIG. 27A loaded into the cartridge jaw of FIG. 27A, where the knife of FIG. 25 is actuated distally from the proximal position such that the pivoting lockout body of FIG. 26 engages a cartridge sled of the staple cartridge;

FIG. 27C is a cross-sectional view of the end effector of FIG. 27A with the staple cartridge of FIG. 27A loaded into the cartridge jaw of FIG. 27A, where the knife of FIG. 25 is actuated distally while the pivoting lockout body of FIG. 26 remains engaged with the cartridge sled of FIG. 27B;

FIG. 28A is a cross-sectional view of the end effector of FIG. 27A without the staple cartridge of FIG. 27A loaded into the cartridge jaw of FIG. 27A, where the knife of FIG. 25 is in the proximal position;

FIG. 28B is a cross-sectional view of the end effector of FIG. 27A without the staple cartridge of FIG. 27A loaded into the cartridge jaw of FIG. 27A, where the knife of FIG. 25 is actuated distally such that the pivoting lockout body contacts a recessed pocket defined by the cartridge jaw, thereby inhibiting further distal actuation of the knife;

FIG. 29 is a perspective view of another alternative knife;

FIG. 30 is a perspective view of another pivoting lockout body that may readily couple with the knife of FIG. 29;

FIG. 31A is a cross-sectional view of another alternative end effector with an unfired staple cartridge loaded into a cartridge jaw of the end effector, where the end effector includes the knife of FIG. 29 and an alternative lockout assembly including the pivoting lockout body of FIG. 30, where the knife is in a proximal position;

FIG. 31B is a cross-sectional view of the end effector of FIG. 31A with the staple cartridge of FIG. 31A loaded into the cartridge jaw of FIG. 31A, where the knife of FIG. 29 is actuated distally from the proximal position such that the pivoting lockout body of FIG. 30 engages a cartridge sled of the staple cartridge;

FIG. 31C is a cross-sectional view of the end effector of FIG. 31A with the staple cartridge of FIG. 31A loaded into the cartridge jaw of FIG. 31A, where the knife of FIG. 29 is actuated distally while the pivoting lockout body of FIG. 31 remains engaged with the cartridge sled of FIG. 31B;

FIG. 32A is a cross-sectional view of the end effector of FIG. 31A without the staple cartridge of FIG. 31A loaded into the cartridge jaw of FIG. 31A, where the knife of FIG. 29 is in the proximal position;

FIG. 32B is a cross-sectional view of the end effector of FIG. 31A without the staple cartridge of FIG. 31A loaded into the cartridge jaw of FIG. 31A, where the knife of FIG. 29 is actuated distally such that the pivoting lockout body contacts a recessed pocket defined by the cartridge jaw, thereby inhibiting further distal actuation of the knife;

FIG. 33 is a perspective view of another alternative knife;

FIG. 34 is a perspective view of another pivoting lockout body that may readily couple with the knife of FIG. 33;

FIG. 35A is a cross-sectional view of another alternative end effector with an unfired staple cartridge loaded into a cartridge jaw of the end effector, where the end effector includes the knife of FIG. 33 and an alternative lockout assembly including the pivoting lockout body of FIG. 34, where the knife is in a proximal position;

FIG. 35B is a cross-sectional view of the end effector of FIG. 35A with the staple cartridge of FIG. 35A loaded into the cartridge jaw of FIG. 35A, where the knife of FIG. 33 is actuated distally from the proximal position such that the pivoting lockout body of FIG. 34 engages a cartridge sled of the staple cartridge;

FIG. 35C is a cross-sectional view of the end effector of FIG. 35A with the staple cartridge of FIG. 35A loaded into the cartridge jaw of FIG. 35A, where the knife of FIG. 33 is actuated distally while the pivoting lockout body of FIG. 34 remains engaged with the cartridge sled of FIG. 35B;

FIG. 36A is a cross-sectional view of the end effector of FIG. 35A without the staple cartridge of FIG. 35A loaded into the cartridge jaw of FIG. 35A, where the knife of FIG. 33 is in the proximal position;

FIG. 36B is a cross-sectional view of the end effector of FIG. 35A without the staple cartridge of FIG. 35A loaded into the cartridge jaw of FIG. 35A, where the knife of FIG. 33 is actuated distally such that the pivoting lockout body contacts a recessed pocket defined by the cartridge jaw, thereby inhibiting further distal actuation of the knife;

FIG. 37 is a perspective view of another knife and pivoting lockout body that may be readily incorporated into the end effector of FIG. 35A in replacement of the knife of FIG. 33 and pivoting lockout body of FIG. 34;

FIG. 38 is a perspective view of another knife and pivoting lockout body that may be readily incorporated into the end effector of FIG. 35A in replacement of the knife of FIG. 33 and pivoting lockout body of FIG. 34;

FIG. 39 is a perspective view of another knife and pivoting lockout body that may be readily incorporated into the end effector of FIG. 35A in replacement of the knife of FIG. 33 and pivoting lockout body of FIG. 34;

FIG. 40 is a perspective view of another pivoting lockout body that may be readily incorporated into the end effector of FIG. 35A in replacement of the pivoting lockout body of FIG. 34;

FIG. 41 is a perspective view of another pivoting lockout body that may be readily incorporated into the end effector of FIG. 35A in replacement of the pivoting lockout body of FIG. 34;

FIG. 42 is a perspective view of another alternative knife;

FIG. 43 is a perspective view of another pivoting lockout body that may readily couple with the knife of FIG. 42;

FIG. 44A is a cross-sectional view of another alternative end effector with an unfired staple cartridge loaded into a cartridge jaw of the end effector, where the end effector includes the knife of FIG. 42 and an alternative lockout assembly including the pivoting lockout body of FIG. 43, where the knife is in a proximal position;

FIG. 44B is a cross-sectional view of the end effector of FIG. 44A with the staple cartridge of FIG. 44A loaded into the cartridge jaw of FIG. 44A, where the knife of FIG. 42 is actuated distally from the proximal position such that the pivoting lockout body of FIG. 43 engages a cartridge sled of the staple cartridge;

FIG. 44C is a cross-sectional view of the end effector of FIG. 44A with the staple cartridge of FIG. 44A loaded into the cartridge jaw of FIG. 44A, where the knife of FIG. 42 is actuated distally while the pivoting lockout body of FIG. 43 remains engaged with the cartridge sled of FIG. 44B;

FIG. 45A is a cross-sectional view of the end effector of FIG. 44A without the staple cartridge of FIG. 44A loaded into the cartridge jaw of FIG. 44A, where the knife of FIG. 42 is in the proximal position;

FIG. 45B is a cross-sectional view of the end effector of FIG. 44A without the staple cartridge of FIG. 44A loaded into the cartridge jaw of FIG. 44A, where the knife of FIG. 42 is actuated distally such that the pivoting lockout body of FIG. 43 contacts a recessed pocket defined by the cartridge jaw, thereby inhibiting further distal actuation of the knife;

FIG. 46 is a perspective view of another alternative knife;

FIG. 47 is a perspective view of another pivoting lockout body that may readily couple with the knife of FIG. 46;

FIG. 48A is a cross-sectional view of another alternative end effector with an unfired staple cartridge loaded into a cartridge jaw of the end effector, where the end effector includes the knife of FIG. 46 and an alternative lockout assembly including the pivoting lockout body of FIG. 47, where the knife is in a proximal position;

FIG. 48B is a cross-sectional view of the end effector of FIG. 48A with the staple cartridge of FIG. 48A loaded into the cartridge jaw of FIG. 48A, where the knife of FIG. 46 is actuated distally from the proximal position such that the pivoting lockout body of FIG. 47 engages a cartridge sled of the staple cartridge;

FIG. 48C is a cross-sectional view of the end effector of FIG. 48A with the staple cartridge of FIG. 48A loaded into the cartridge jaw of FIG. 48A, where the knife of FIG. 46 is actuated distally while the pivoting lockout body of FIG. 47 remains engaged with the cartridge sled of FIG. 48B;

FIG. 49A is a cross-sectional view of the end effector of FIG. 48A without the staple cartridge of FIG. 48A loaded into the cartridge jaw of FIG. 48A, where the knife of FIG. 46 is in the proximal position;

FIG. 49B is a cross-sectional view of the end effector of FIG. 48A without the staple cartridge of FIG. 48A loaded into the cartridge jaw of FIG. 48A, where the knife of FIG. 46 is actuated distally such that the pivoting lockout body of FIG. 47 contacts a recessed pocket defined by the cartridge jaw, thereby inhibiting further distal actuation of the knife;

FIG. 50 is a perspective view of another alternative knife and lockout body attached to knife;

FIG. 51A is a cross-sectional view of another alternative end effector with an unfired staple cartridge loaded into the cartridge jaw of the end effector, where the end effector includes the knife of FIG. 50 and an alternative lockout assembly including the lockout body of FIG. 50, where the knife is in a proximal position;

FIG. 51B is a cross-sectional view of the end effector of FIG. 51A with the staple cartridge of FIG. 51A loaded into the cartridge jaw of FIG. 51A, where the knife of FIG. 50 is actuated distally from the proximal position such that the lockout body of FIG. 50 engages a carriage sled of the staple cartridge;

FIG. 51C is a cross-sectional view of the end effector of FIG. 51A with the staple cartridge of FIG. 51A loaded into the cartridge jaw of FIG. 51A, where the knife of FIG. 50 is actuated distally while the lockout body of FIG. 50 remains engaged with the cartridge sled of FIG. 51B;

FIG. 52A is a cross-sectional view of the end effector of FIG. 51A without the staple cartridge of FIG. 51A loaded into the cartridge jaw of FIG. 51A, where the knife of FIG. 50 is in the proximal position;

FIG. 52B is a cross-sectional view of the end effector of FIG. 51A without the staple cartridge of FIG. 51A loaded into the cartridge jaw of FIG. 51A, where the knife of FIG. 50 is actuated distally such that the lockout body of FIG. 50 contacts a recessed pocket defined by the cartridge jaw, thereby inhibiting further distal actuation of the knife;

FIG. 53 is an exploded view of an alternative end effector having a knife lockout assembly;

FIG. 53A is a cross-sectional view of an anvil jaw of the end effector of FIG. 53;

FIG. 54A is a cross-sectional view of the end effector of FIG. 53 with an unfired staple cartridge loaded into the cartridge jaw of the end effector, where a knife of the end effector is in a proximal position;

FIG. 54B is a cross-sectional view of the end effector of FIG. 53 with the staple cartridge of FIG. 54A loaded into the cartridge jaw of FIG. 54A, where the knife of FIG. 54A is actuated distally from the proximal position such that a lockout body of the knife lockout assembly of FIG. 53 engages a cartridge sled of the staple cartridge and such that the lockout body releases the knife;

FIG. 54C is a cross-sectional view of the end effector of FIG. 53 with the staple cartridge of FIG. 54A loaded into the cartridge jaw of FIG. 54A, wherein the knife of FIG. 54A is actuated distally past the lockout body of FIG. 54B;

FIG. 55A is a cross-sectional view of the end effector of FIG. 53 without the staple cartridge of FIG. 54A loaded into the cartridge jaw of FIG. 54A, where the knife of FIG. 54A is in the proximal position;

FIG. 55B is a cross-sectional view of the end effector of FIG. 53 without the staple cartridge of FIG. 54A loaded into the cartridge jaw of FIG. 54A, where the knife of FIG. 54A is actuated distally such that the lockout body of FIG. 54B engages the knife, thereby inhibiting further distal actuation off the knife;

FIG. 56 is an exploded view of an alternative end effector having a knife lockout assembly;

FIG. 57 is a perspective view of the knife lockout assembly of FIG. 56;

FIG. 58 is another perspective view of the knife lockout assembly of FIG. 56;

FIG. 59A is a cross-sectional view of the end effector of FIG. 56 with an unfired staple cartridge loaded into the cartridge jaw of the end effector, where a knife of the end effector is in a proximal position;

FIG. 59B is a cross-sectional view of the end effector of FIG. 56 with the staple cartridge of FIG. 59A loaded into the cartridge jaw of FIG. 59A, where the knife of FIG. 59A is actuated distally from the proximal position such that a lever of the knife lockout assembly of FIG. 56 engages a cartridge sled of the staple cartridge and such that a lockout hook of the knife lockout assembly is disengaged with the knife;

FIG. 59C is a cross-sectional view of the end effector of FIG. 56 with the staple cartridge of FIG. 59A loaded into the cartridge jaw of FIG. 59A, where the knife of FIG. 59A is actuated distally past the lockout hook of FIG. 59B;

FIG. 60A is a cross-sectional view of the end effector of FIG. 56 without the staple cartridge of FIG. 59A loaded into the cartridge jaw of FIG. 59A, where the knife of FIG. 9A is in the proximal position; and

FIG. 60B is a cross-sectional view of the end effector of FIG. 56 without the staple cartridge of FIG. 59A loaded into the cartridge jaw of FIG. 59A, where the knife of FIG. 59A is actuated distally such that the lockout hook of FIG. 59B engages the knife, thereby inhibiting further distal actuation off the knife.

### DETAILED DESCRIPTION

The following detailed description should be read with reference to the drawings, in which like elements in different drawings are identically numbered. The drawings, which are not necessarily to scale, depict selected versions and are not intended to limit the scope of the invention. The detailed description illustrates by way of example, not by way of limitation, the principles of the invention. This description will clearly enable one skilled in the art to make and use the invention, and describes several versions, adaptations, variations, alternatives and uses of the invention, including what is presently believed to be the best mode of carrying out the invention.

Numerous specific details are set forth to provide a thorough understanding of the overall structure, function, manufacture, and use of the versions as described in the specification and illustrated in the accompanying drawings. Well-known operations, components, and elements have not been described in detail so as not to obscure the versions described in the specification. The reader will understand that the versions described and illustrated herein are non-limiting examples, and thus it can be appreciated that the specific structural and functional details disclosed herein may be representative and illustrative. Variations and changes thereto may be made without departing from the scope of the claims.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a surgical system, device, or apparatus that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

The terms “proximal” and “distal” are used herein with reference to a robotic platform manipulating the housing portion of the surgical instrument. The term “proximal” refers to the portion closest to the robotic platform and the term “distal” refers to the portion located away from the robotic platform. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Furthermore, the terms “about,” “approximately,” “substantially,” and the like as used herein in connection with any numerical values, ranges of values, and/or geometric/positional quantifications are intended to encompass the exact value(s) or quantification(s) referenced as well as a suitable tolerance that enables the referenced feature or combination of features to function for the intended purpose described herein. For example, “substantially parallel” encompasses nominally parallel structures, and “substantially equal” values encompass nominally equal values.

Furthermore, the use of “couple”, “coupled”, or similar phrases should not be construed as being limited to a certain number of components or a particular order of components unless the context clearly dictates otherwise.

### I. Overview of Illustrative Surgical Instrument

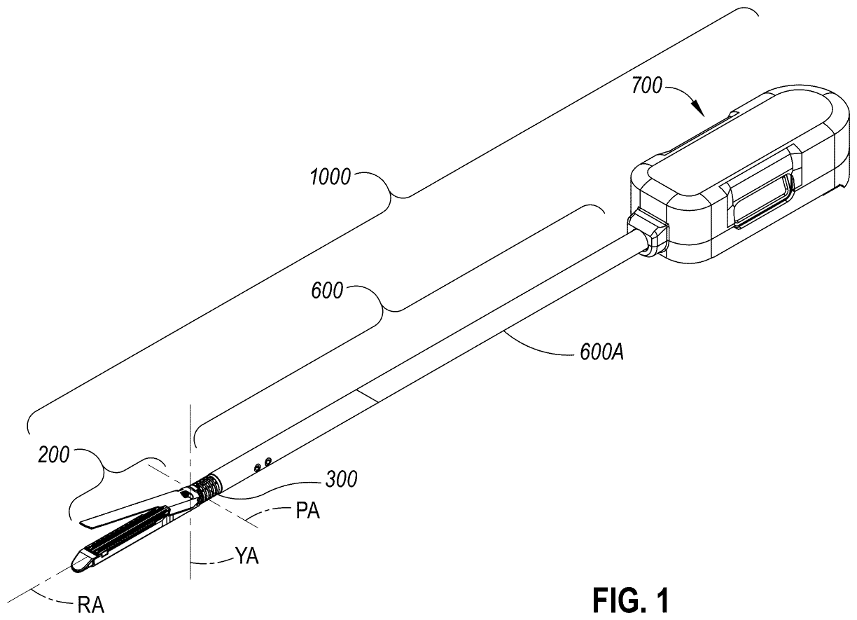

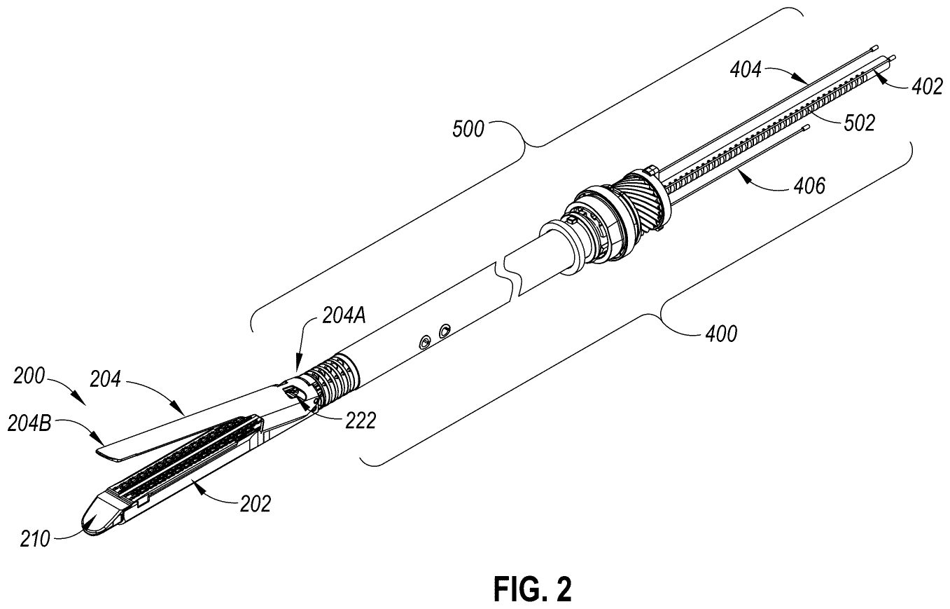

FIGS. 1-2 show an illustrative surgical instrument 1000 that is configured to grasp, clamp, incise, and seal patient tissue with staples. The surgical instrument 1000 comprises an end effector 200, an articulation joint 300 (also referred to as a “continuum joint”), an articulation drive subsystem 400 configured to articulate the end effector 200 via the articulation joint 300, a knife firing subsystem 500 configured to actuate the end effector 200 between various positions (e.g., an open position, a grasping position, and a clamping position) and to incise and staple patient tissue, a roll subsystem 600 configured to rotate the end effector 200 about a roll axis RA, and a housing 700.

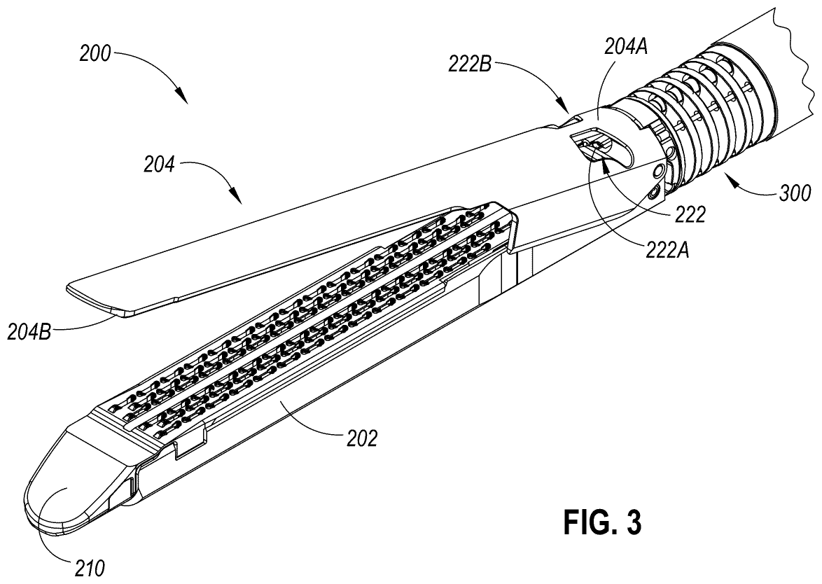

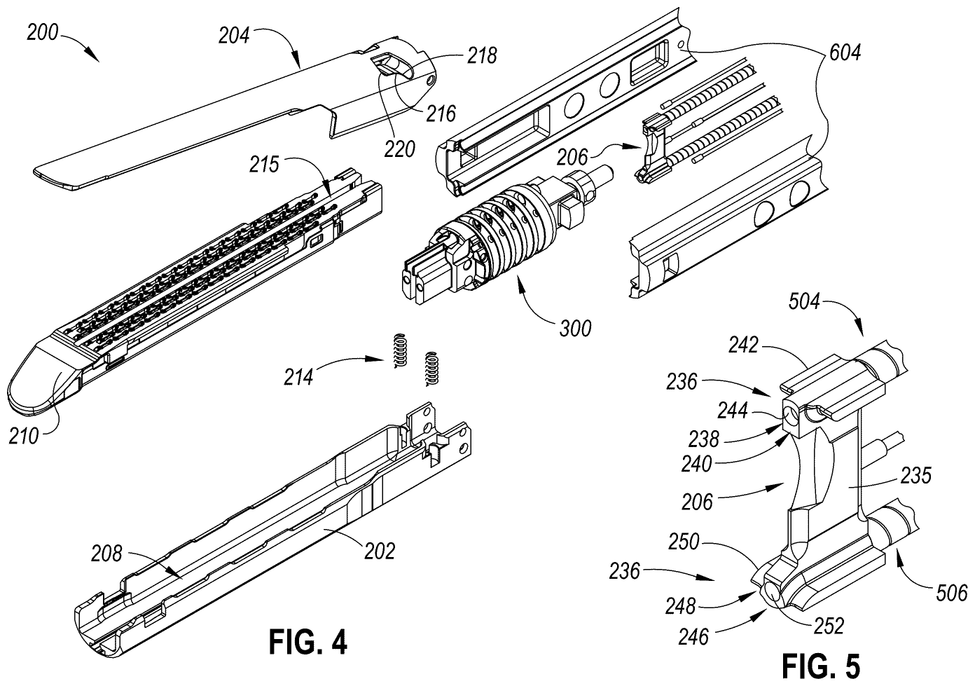

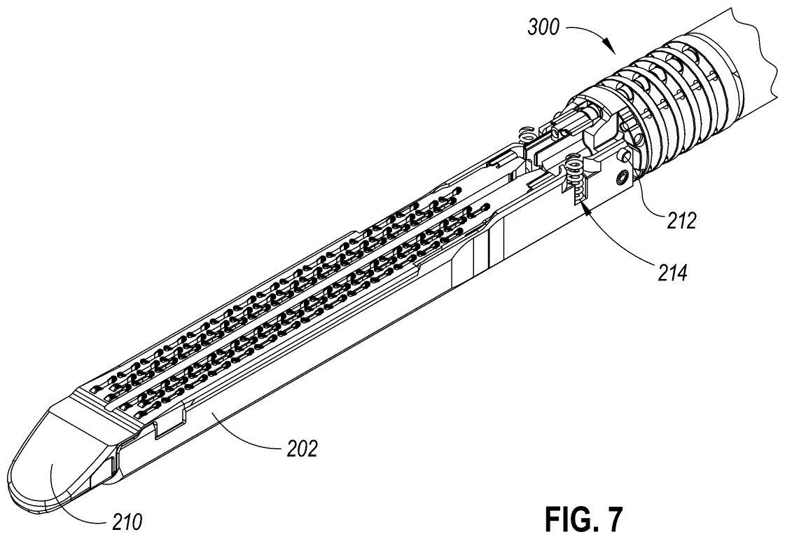

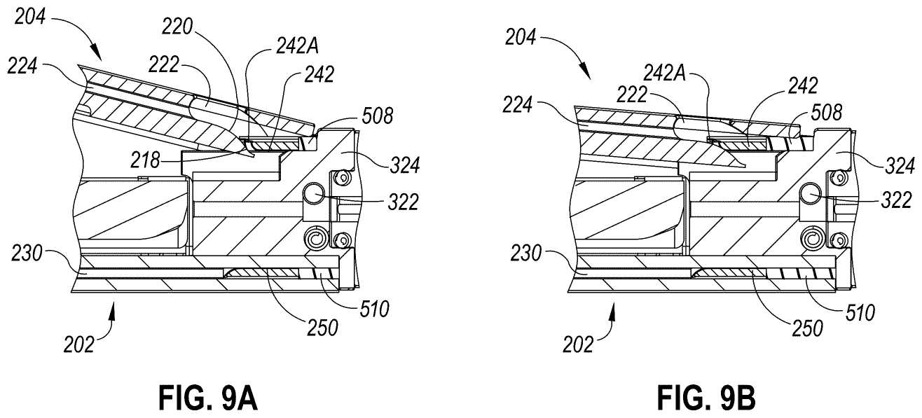

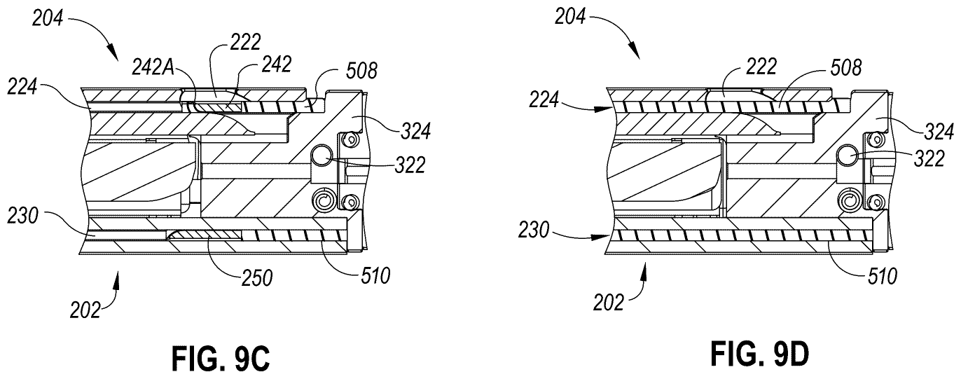

As shown best in FIGS. 3-4, the end effector 200 comprises a first jaw 202 (also known as a “cartridge jaw” or a “channel”) and a second jaw 204 (also known as an “anvil jaw” or just “anvil”) movable relative to the cartridge jaw 202 between an open position and a closed position. The cartridge jaw 202 and anvil jaw 204 may be elongated in form. The cartridge jaw 202 defines an elongated channel 208 for receiving a staple cartridge 210 (also known as a “reload”). The anvil jaw 204 has a proximal end 204A, a distal end 204B, and a ramp surface 216 defined at the proximal end 204A, which is described in greater detail below with respect to FIGS. 4 and 9A-9D. The cartridge jaw 202 and anvil jaw 204 are pivotally coupled via a pivot pin 212 that extends through the cartridge jaw 202 and the anvil jaw 204. As seen in FIG. 7, one or more biasing springs 214 extend between the cartridge jaw 202 and anvil jaw 204 to bias the anvil jaw 204 to the open position.

The ramp surface 216 may be visible via a kidney bean-shaped opening 222 (which may be formed as part of the manufacturing process to make the ramp surface 216) that has a first lateral end 222A and a second lateral end 222B. In other words, the kidney bean-shaped opening may be open at its lateral ends 222A, 222B (FIG. 3). As seen in FIG. 4, the ramp surface 216 forms a lower surface of the kidney bean-shaped opening 222. The ramp surface 216 can be arcuately shaped. For example, as shown particularly in FIGS. 4 and 9A-9D, it may be upwardly sloped at a first angle 218 and arcuately taper, in a distal direction, to a substantially horizontal second angle 220.

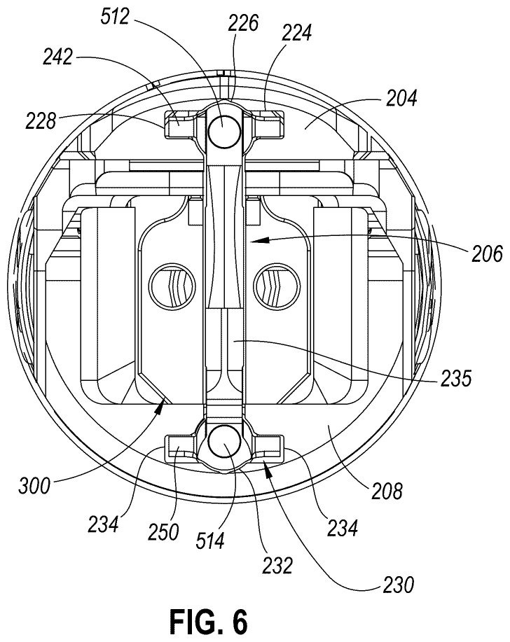

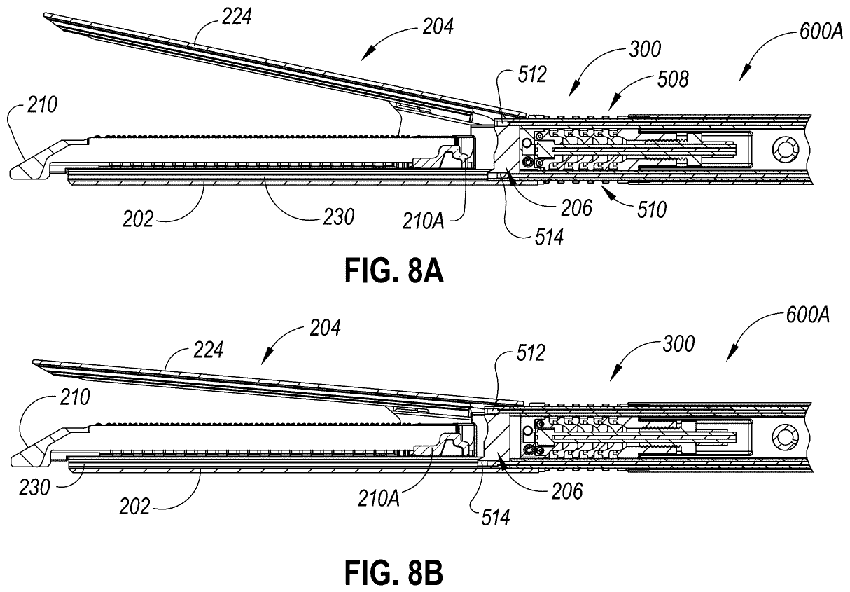

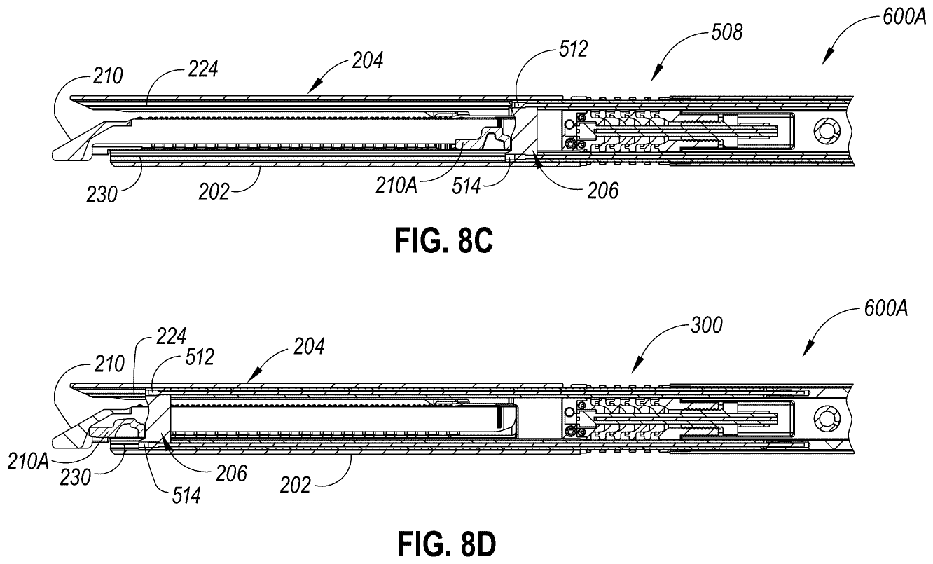

The anvil jaw 204 further defines a longitudinally extending upper knife channel 224 (see FIG. 8A, etc.). As shown particularly in FIG. 6, the upper knife channel 224 includes a centrally disposed cylindrical upper knife channel portion 226 and at least one lateral upper knife channel wing 228 that extends away from the upper knife channel portion 226. While the term ‘cylindrical’ is used, the channel portion 226 need not resemble a perfect cylinder.

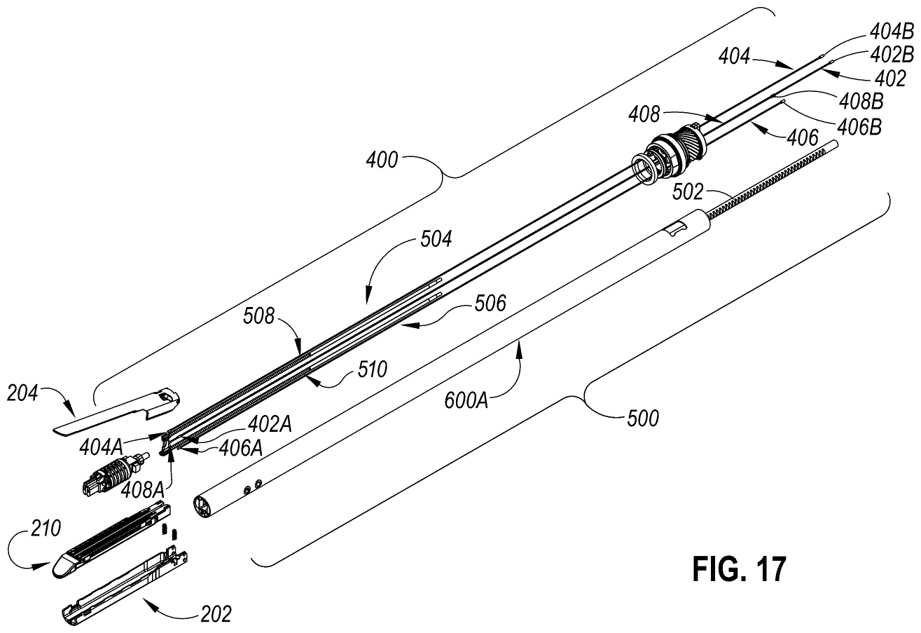

As shown in FIGS. 2 and 17, the surgical instrument 1000 further comprises a knife firing subsystem 500 operable to close the anvil jaw 204 during a closure stroke. After the end effector 200 is closed, the knife firing subsystem 500 is operable to incise and staple, with staples from the staple cartridge 210, the patient tissue captured between the staple cartridge 210 (which is retained by the cartridge jaw 202) and anvil jaw 204 during a firing stroke.

As shown best in FIGS. 4-6, the knife firing subsystem 500, explained further below in greater detail, includes a knife 206 having a knife sled 236. The knife sled 236 functions as a firing driver by driving cartridge sled 210A distally through a firing stroke, as described below. In some instances, knife sled 236 may be referred to as an I-beam. The knife sled 236 includes an upper knife tab 238, a lower knife tab 246, and a vertical column 235 coupling and extending between upper knife tab 238 and lower knife tab 246. The upper knife tab 238 includes a centrally disposed cylindrical upper knife tab portion 240 and at least one upper knife tab lateral wing 242 that extends away from the upper knife tab portion 240. While the term ‘cylindrical’ is used, the tab portion need not resemble a perfect cylinder.

The upper knife tab 238 may include a pair of lateral wings 242 configured to slidably ride in the upper knife channel 224 to move the anvil jaw 204 between the open position, the grasping position, and the clamping position. Accordingly, the end effector 200 employs “knife-based closure” in which closure of the anvil jaw 204 relative to the cartridge jaw 202 is driven by distal advancement of the knife 206. Each lateral wing 242 may include a ramped surface 242A that engages the anvil ramp surface 216. The upper knife tab portion 240 defines an upper knife tab opening 244 that is configured to receive a barrel crimp coupled to a center cable 512, which is described in greater detail below. The lower knife tab 246 includes a centrally disposed cylindrical lower knife tab portion 248 and at least one lower knife tab lateral wing 250 that extends away from the lower knife tab portion 248. While the term ‘cylindrical’ is used, the lower knife tab portion 248 need not resemble a perfect cylinder. In some versions, the lower knife tab 246 includes a pair of lateral wings 250. The lower knife tab portion 248 defines a lower knife tab opening 252 that is configured to receive a barrel crimp coupled to a center cable 514, as described in greater detail below.

The staple cartridge 210 may be generally constructed and operable in accordance with the teachings of U.S. patent application Ser. No. 18/588,684, entitled “Methods of Surgical Stapling,” filed on Feb. 27, 2024, the disclosure of which is incorporated by reference herein in its entirety. In use, the end effector 200 is positioned relative to patient tissue such that the staple cartridge 210 is disposed on a first side of the tissue and the anvil jaw 204 is positioned on an opposed second side of the tissue. The anvil jaw 204 is then approximated toward the staple cartridge 210 to compress and clamp the tissue against the deck of the staple cartridge 210. Thereafter, the surgical instrument 1000 is fired so that the knife 206 advances distally through the staple cartridge 210 to both cut the clamped tissue and simultaneously actuate staple drivers housed within the staple cartridge 210 to drive an array of staples into the clamped tissue on either side of the cut line. Staple cartridge 210 defines an elongate knife channel 215 dimensioned to receive a portion of vertical column 235 in order to accommodate advancement of knife 206 through staple cartridge 210. A portion of cartridge sled 210A is slidably housed within elongate knife channel 215 such that vertical column 235 drives cartridge sled 210A distally as knife 206 advances distally in accordance with the description herein (see FIGS. 8C-8D). In some instances, cartridge sled 210A remains in the distal position (see FIG. 8D) relative to the rest of staple cartridge 210, even after knife 206 is retracted proximally after firing staple cartridge 210 in accordance with the description herein.

As mentioned above, cartridge jaw 202 defines an elongated channel 208 for receiving staple cartridge 210. Additionally, cartridge jaw 202 also defines a lower knife channel 230 (see FIGS. 4, 6, and 8A-9D) dimensioned to slidably receive lower knife tab 246. Referring to FIG. 6, the lower knife channel 230 includes a centrally disposed cylindrical lower knife channel portion 232 and at least one lateral lower knife channel wing 234 that extends away from the lower knife channel portion 232. Cylindrical lower knife channel portion 232 is in communication with elongated channel 208 such that when staple cartridge 210 is suitably coupled to cartridge jaw 202, elongate knife channel 215 of staple cartridge 210 and centrally disposed cylindrical lower knife channel portion 232 are aligned to accommodate actuation of knife sled 236 within both channels 215, 230. Lateral lower knife channel wings 234 are dimensioned to slidably house a respective lower knife tab lateral wing 250. Lower knife tab lateral wings 250 are configured to slidably contact lateral lower knife channel wings 234 as knife 206 is advanced in accordance with the description herein. Contact between lower knife tab lateral wings 250 and lateral lower knife channel wings 234 cooperatively assists lateral wings 242 and upper knife channel 224 to close anvil jaw 204 relative to channel 208 in accordance with the description herein. While the term ‘cylindrical’ is used, the channel portion 232 need not resemble a perfect cylinder. Other arrangements of staple cavities and staples may be possible. For example, in some versions, a lower knife channel 230 can be defined in the cartridge jaw 202.

Further to the above, the knife sled 236 is moved distally and proximally by a firing rod 502. The firing rod 502 is configured to apply an indirect force to the knife sled 236, via push coils 508, 510 that directly engage the knife sled 236 (discussed in greater detail below), and push the knife sled 236 toward the distal end of the end effector 200 through a firing stroke. As the firing rod 502 is advanced distally, knife sled 236 rides in the lower knife channel 230 and the upper knife channel 224. At the onset of travel, the upper knife tab 238 rides along the anvil ramp surface 216. Specifically, as particularly seen in the sequence of FIGS. 8A-8D and 9A-9D, movement of the knife sled 236 distally causes the upper knife tab ramped surface 242A to slide along the anvil ramp surface 216. This movement first urges the anvil jaw 204 closed to a position (e.g., FIGS. 8B and 9B) where a compressive force is applied to the tissue sufficient to grasp it (referred to as the grasping position). Continued movement of the knife sled 236 up the ramp surface 216 (e.g., see FIGS. 8C and 9C) results in a compressive force being applied to the tissue (referred to as the clamping position). As the anvil ramp surface 216 transitions to its substantially horizontally angled surface 220 (e.g., see FIGS. 8D and 9D), the upper knife tab 238 can slide within the upper knife channel 224 to drive the stapling and transection of the tissue.

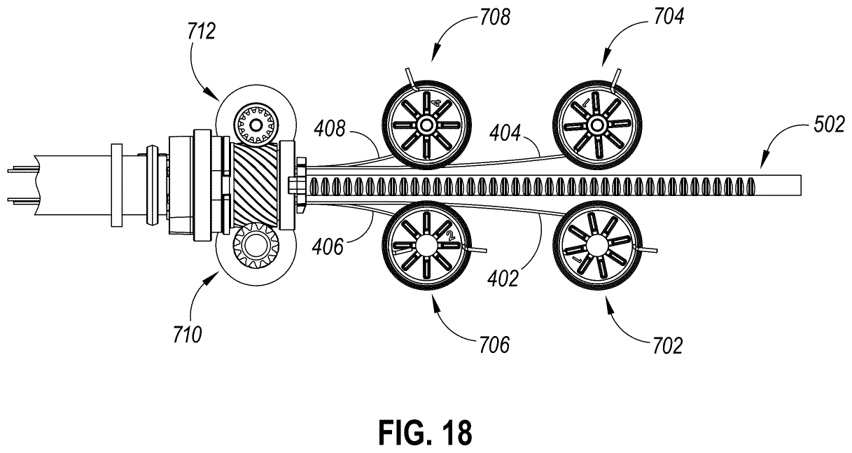

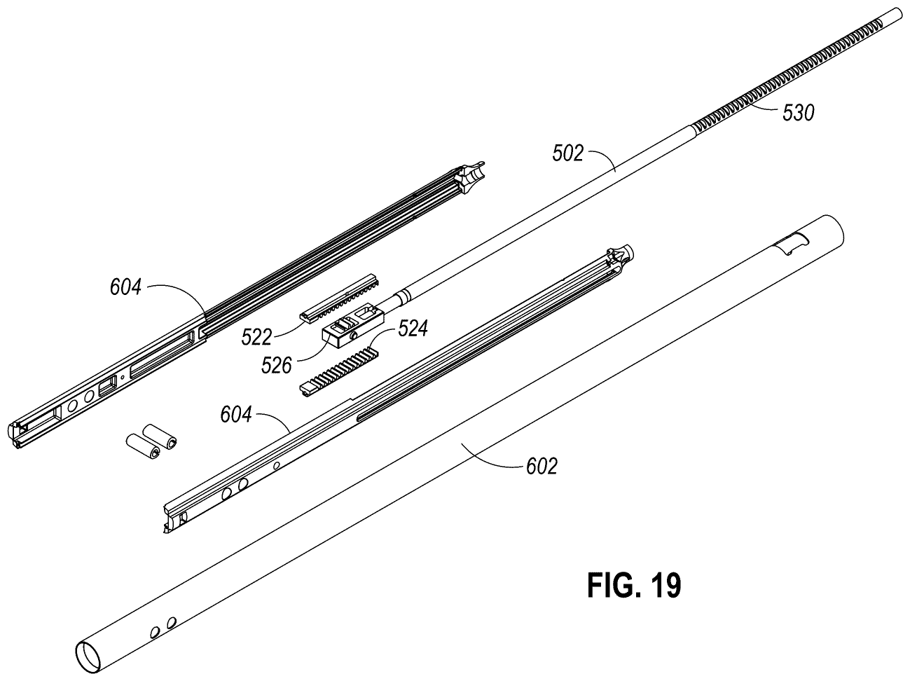

As shown in FIG. 1, the surgical instrument 1000 further comprises a body exemplified as a housing 700 configured to engage a robotic platform (not shown). In other versions, the body may be configured as a handle configured to be gripped and manipulated by a clinician. As best shown in FIGS. 1 and 19, a shaft assembly 600A extends distally from the housing 700 and includes a rotatable outer shaft 602 and an inner shaft 604 arranged in two clamshell halves, with the outer shaft 602 being rotatably mounted to the housing 700 about a rotation joint (not shown), which may include one or more bearings. The inner shaft 604 is rotationally fixed to the outer shaft 602 and is configured such that articulation cables 402, 404, 406, 408 can be partially wound therearound without becoming tangled. As shown in FIG. 18, the housing 700 may house (1) a firing puck assembly 712 as part of the knife firing subsystem 500 operable to close the end effector 200, fire staples, and transect tissue, (2) a set of articulation puck assemblies 702, 704, 706, 708 as part of the articulation subsystem 400 operable to articulate the end effector 200 relative to the shaft assembly 600A, and (3) a shaft roll puck assembly 710 as part of the roll subsystem 600 configured to roll the outer shaft 602.

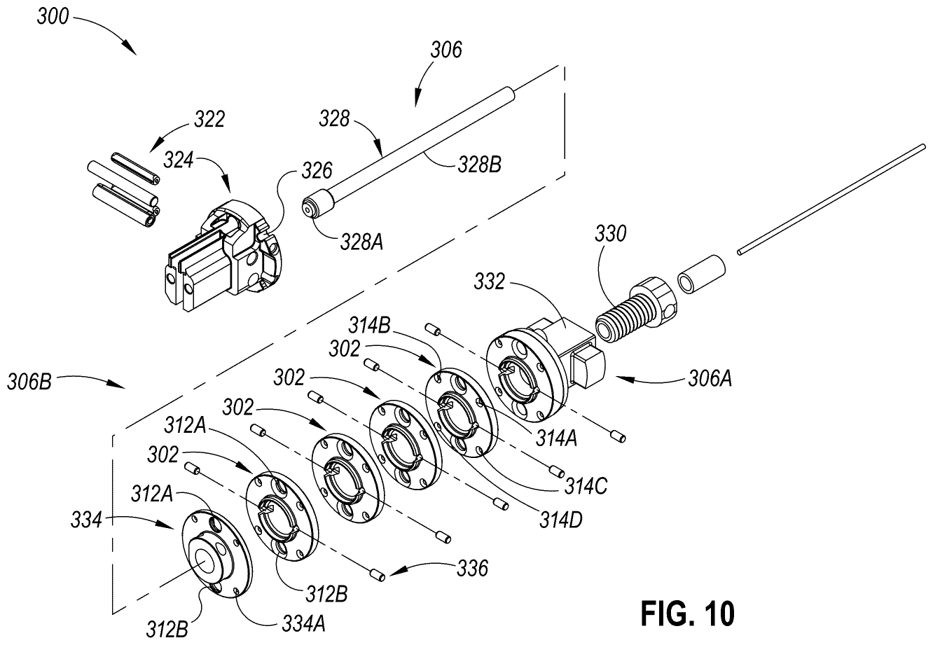

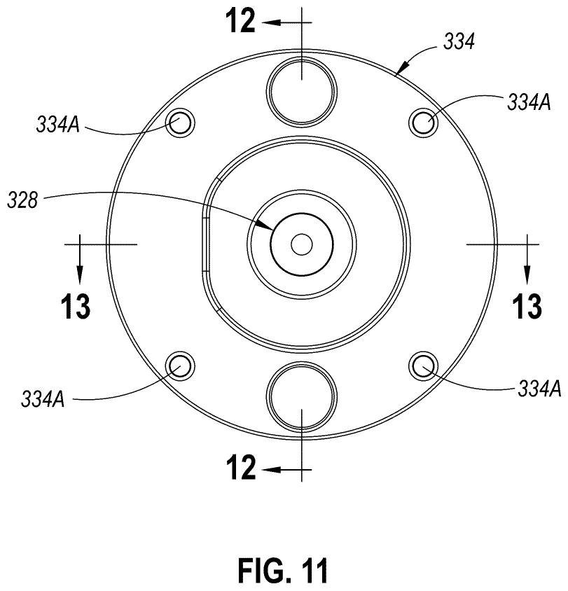

Referring to FIGS. 10-13, the articulation joint 300 comprises an array of joint discs 302 arranged longitudinally, and a center beam assembly 306 that cooperates with the joint discs 302 to provide articulation of the end effector 200 with at least two degrees of freedom (e.g., yaw and pitch), as described further below. Each joint disc 302 includes a central opening 304 that is configured to align coaxially with the central opening 304 of the other joint discs when the articulation joint 300 is in a straight, non-articulated state. The center beam assembly 306 extends longitudinally through the central openings 304 of joint discs 302 and applies a compressive axial force to the array of joints discs 302 to couple the joint discs 302 with one another. The joint discs 302 are nestably stacked with one another along the center beam assembly 306 such that longitudinally adjacent joint discs 302 movably interface with one another.

As seen in FIGS. 9A-10, a distal end 306B of the center beam assembly 306 includes a distal retainer 324 that couples the distal end of the articulation joint 300 with a proximal end of the cartridge jaw 202 via one or more fasteners 322, thereby mechanically grounding and retaining the cartridge jaw 202 and thus the end effector 200 relative to the articulation joint 300. The distal retainer 324 includes a plurality of clearance pockets 326 that receive distal ends of articulation cables 402, 404, 406, 408. The distal end 306B further includes a distal retention disc 334 that defines a plurality of cable retention openings 334A. A proximal end 306A of the center beam assembly 306 includes a proximal retainer 332 that couples the proximal end of the articulation joint 300 with a distal end of the shaft assembly 600A.

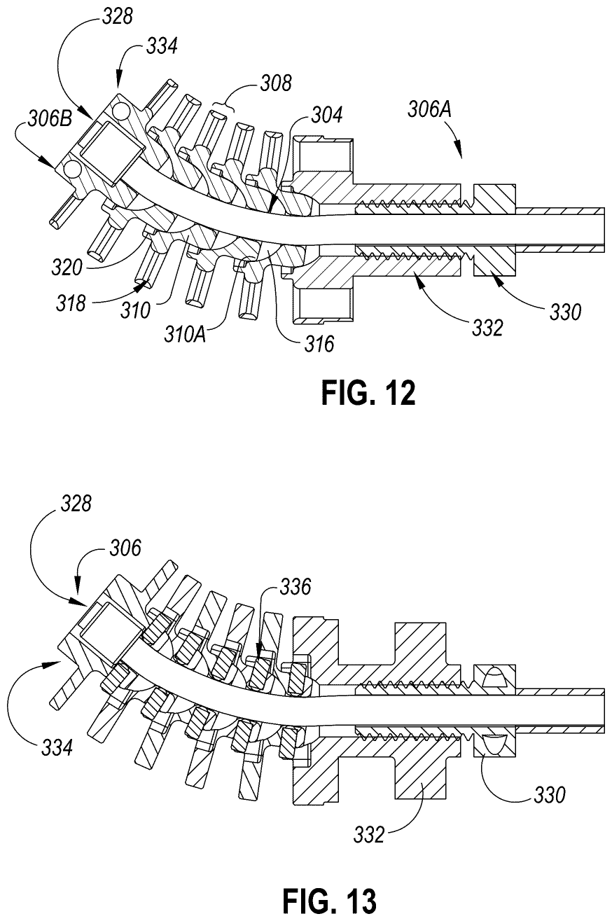

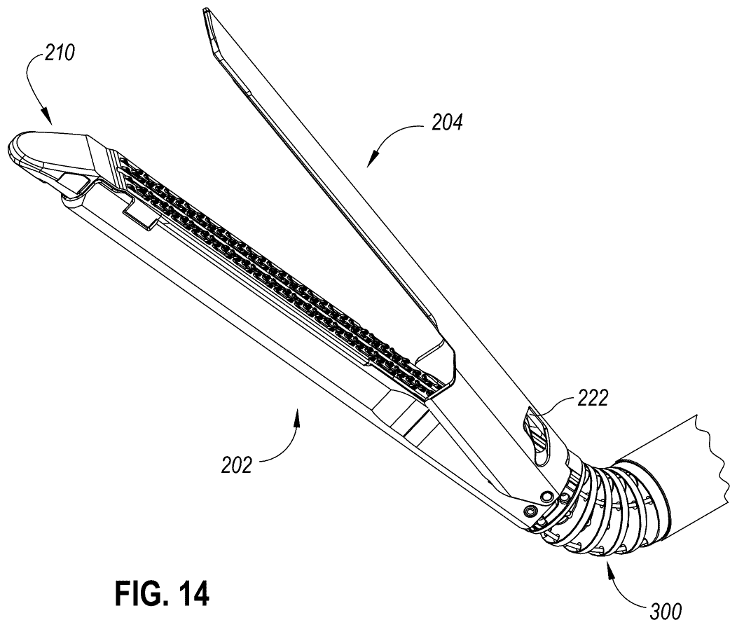

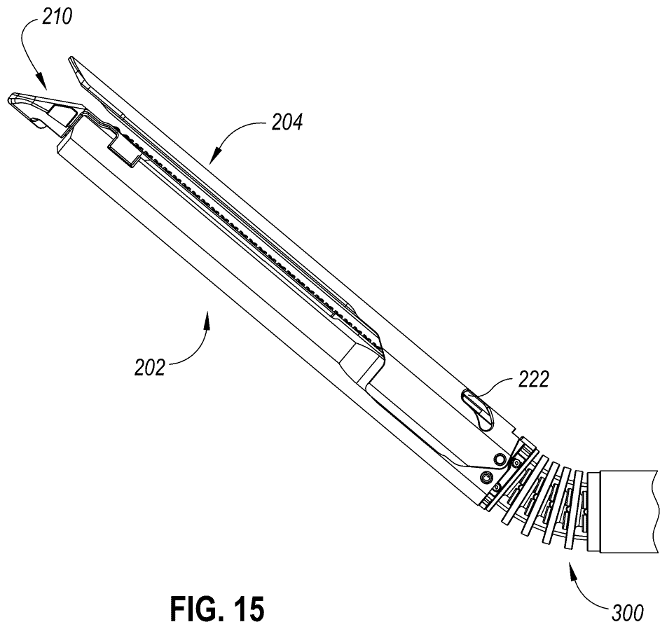

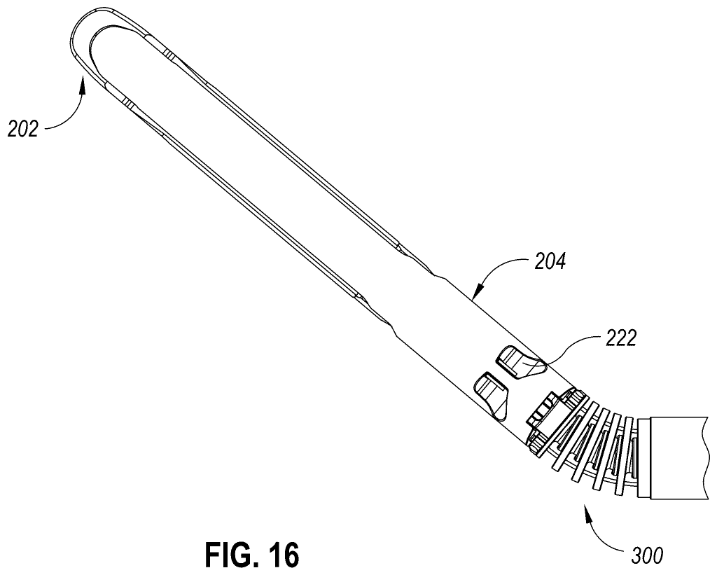

As shown particularly in FIGS. 10, 12, and 13, each joint disc 302 includes an articulation socket 308, an articulation pin 310 protruding outwardly from the articulation socket 308, a first push coil opening 312A defined through the articulation socket 308 and configured to receive a first push coil 508 therethrough, a second push coil opening 312B defined through the articulation socket 308 and configured to receive a second push coil 510 therethrough, and a plurality of articulation cable openings 314A-314D (e.g., a first articulation cable opening 314A, a second articulation cable opening 314B, a third articulation cable opening 314C, and a fourth articulation cable opening 314D) defined through the articulation socket 308 and configured to receive a respective articulation cable 402, 404, 406, 408 (e.g., a first articulation cable 402, a second articulation cable 404, a third articulation cable 406, and a fourth articulation cable 408) therethrough, and discussed in greater detail below. As shown in FIGS. 12 and 13, the central opening 304 is defined in the articulation pin 310 of each joint disc 302. In some versions, three articulation cable openings 314A, 314B, 314C are provided to correspond to three articulation cables 402, 404, 406, while in other versions, four articulation cable openings 314A, 314B, 314C, 314D are provided to correspond to four articulation cables 402, 404, 406, 408.

Each joint disc 302 further includes a rounded articulation pin proximal end 310A and a semi-spherical pin-receiving opening 316 defined in the articulation socket 308. As shown particularly in FIGS. 12 and 13, each rounded articulation pin proximal end 310A pivotally engages in an adjacent pin-receiving opening 316 of an adjacent joint disc 302, with the exception of a proximal-most end 310A that engages with the proximal retainer 332. The articulation pin proximal end 310A and pin-receiving opening 316 interface functions in a similar manner as a swivel bearing. Moreover, the articulation socket 308 includes a socket disc 318 and a pin retention socket 320. A pair of pins 336 are used to provide rotational coupling about a primary axis of the shaft assembly 600A from one disc 302 to the next. In other words, the pins constrain a rotational degree of freedom between adjacent joint discs 302 about the roll axis RA of the instrument 1000. In alternative versions, this feature can be integral to the joint disc 302.

The center beam assembly 306 further includes a center beam 328 that extends longitudinally through the central openings 304 of the joint discs 302. The center beam 328 includes a nitinol core 328A and a stainless-steel collar 328B wound over the nitinol core 328A that allows the center beam 328 to resiliently flex during deflection of the articulation joint 300. The wound stainless-steel collar 328B may have clockwise braiding and counterclockwise braiding to prevent unwinding thereof. The center beam assembly further includes a jack screw 330 that is threadably coupled with the proximal retainer 332 to adjust an axial compression force exerted by the center beam 328 on the array of joint discs 302, thereby enabling adjustment of a pre-load of the articulation joint 300.

The above-described articulation joint 300 forms a portion of the cable articulation subsystem 400 which allows for precise 360-degree movement of the end effector 200 about the articulation joint 300 with at least two degrees of freedom. In some versions, and as dictated by the roll subsystem 600 as well as a need to limit the amount of wrap of the articulation cables 402, 404, 406, 408, the articulation joint 300 is permitted about 320 degrees of roll within the overall system. The cable articulation subsystem 400 also includes a plurality of articulation cables 402, 404, 406, 408 each having a distal end 402A, 404A, 406A, 408A, coupled to the distal end 306B of the center beam assembly 306, and a proximal end 402B, 404B, 406B, 408B. More specifically, each distal end 402A, 404A, 406A, 408A can include a crimp that engages a cable retention opening 334A of the distal retention disc 334 to maintain its positioning. Each articulation cable is discretely manipulable to cause rotation of the articulation joint 300 and end effector 200 about at least one of a pitch axis PA and a yaw axis YA.

In some versions, three articulation cables may be provided rather than the four cables 402, 404, 406, 408 depicted herein. However, four articulation cables 402, 404, 406, 408 circumferentially spaced approximately ninety degrees from one another (as shown) provide load splitting. Additionally, in alternative versions, three and fourth articulation cable configurations may be spaced non-symmetrically relative to one another.

The shaft assembly 600A and housing 700 also form portions of the cable articulation subsystem 400. More specifically, each articulation cable 402, 404, 406, 408 extends from the articulation joint 300 and through the shaft assembly 600A to the housing 700. The proximal end 402B, 404B, 406B, 408B of each articulation cable (402, 404, 406) is movably mounted in the housing 700 which causes the above-mentioned rotation of the articulation joint 300 and end effector 200. The housing 700 includes articulation puck assemblies 702, 704, 706, 708 with rotatable capstans (not shown) about which corresponding proximal ends 402B, 404B, 406B, 408B of the articulation cables 402, 404, 406, 408 are windably mounted.

The articulation cables 402, 404, 406, 408 are routed through the shaft assembly 600A such that they are disposed between the outer shaft 602 and the inner shaft 604, with the articulation cables 402, 404, 406, 408 being able to partially wind therearound without becoming tangled. The inner shaft 604 also prevents the articulation cables 402, 404, 406, 408 from interfering with other components running down the center of the instrument 1000 (through the inner shaft 604).

The articulation cables 402, 404, 406, 408 are routed and coupled to the end effector 200 via the articulation joint 300 such that movement thereof in a proximal direction (via winding about the capstans of the housing 700) causes the end effector 200 to articulate in a predetermined manner via the articulation joint 300. For example, actuation of the first articulation cable 402 in the proximal direction causes articulation of the end effector 200 upwards and to the left, actuation of the second articulation cable 404 in the proximal direction causes rotation of the end effector 200 upwards and to the right, actuation of the third articulation cable 406 in the proximal direction causes rotation of the end effector 200 downwards and to the left, and actuation of the fourth articulation cable 408 in the proximal direction causes rotation of the end effector 200 downwards and to the right. Similarly, movement of two articulation cables simultaneously will result in blended articulation of the end effector 200. As will be appreciated by those skilled in the art, this configuration provides for the above-mentioned precise 360-degree articulation of the end effector 200 via the articulation joint 300 with at least two degrees of freedom and about 320 degrees of roll.

As shown throughout FIGS. 2, 4, 5, 8A-8D, 9A-9D, 17 and 19, the knife firing subsystem 500 includes the aforementioned knife 206, the aforementioned knife sled 236, a firing rod 502 that drives the knife 206 and/or knife sled 236, a first push rod 504, and a second push rod 506. The firing rod 502 includes a firing rod rack 530 and is driven by a firing puck assembly 712 of the housing 700. The first push rod 504 has a first push rod distal end 504A coupled to the knife sled 236 and a first push rod proximal end 504B coupled to the firing rod 502. Similarly, the second push rod has a second push rod distal end 506A coupled to the knife sled 236 and a second push rod proximal end 506B coupled to the firing rod 502. The distal ends 504A, 506A are coupled to respective upper and lower portions of the knife sled 236 (e.g., the upper knife tab 238 and the lower knife tab 246), which enables the knife 206 to be pushed evenly at its ends. In some versions, the proximal ends 504B, 506B of the push rods 504, 506 are coupled to the firing rod 502 via a differential 520.

The knife firing subsystem 500 is configured in a manner to enable articulation of the end effector 200 while still enabling proper functionality of the knife 206. To that end, the first push rod 504 includes a first flexible section in the form of a first push coil 508 and the second push rod 506 comprises a second flexible section in the form of a second push coil 510. The push coils 508, 510 route through the articulation joint 300 via the respective push coil openings 312A, 312B, and the push rods 504, 506 engage the respective tab openings 244, 252 in the knife sled 236. A first center cable 512 extends through the first push coil 508 to engage the knife sled 236 via a barrel crimp, and a second center cable 514 extends through the second push coil 510 to engage the knife sled 236 via a barrel crimp. The push coils 508, 510 provide the push rods 504, 506 sufficient stability to deliver an axial firing force to the knife 206, while not being too stiff that would prevent articulation at the joint 300. The cables 512, 514, which are engaged with the knife sled 236 as discussed above (see, e.g., FIG. 8A), prevent the push coils 508, 510 from stretching and/or elongating and serve as retraction cables when the rods 504, 506 are retracted towards the proximal end of the surgical instrument 1000. The entirety of each push rod 504, 506 does not extend through the articulation joint 300, and therefore does not need to be flexible. Accordingly, a proximal section of each push rod 504, 506 can be less flexible than the push coils 508, 510.

### II. Illustrative Lockouts for Knife Firing Subsystem

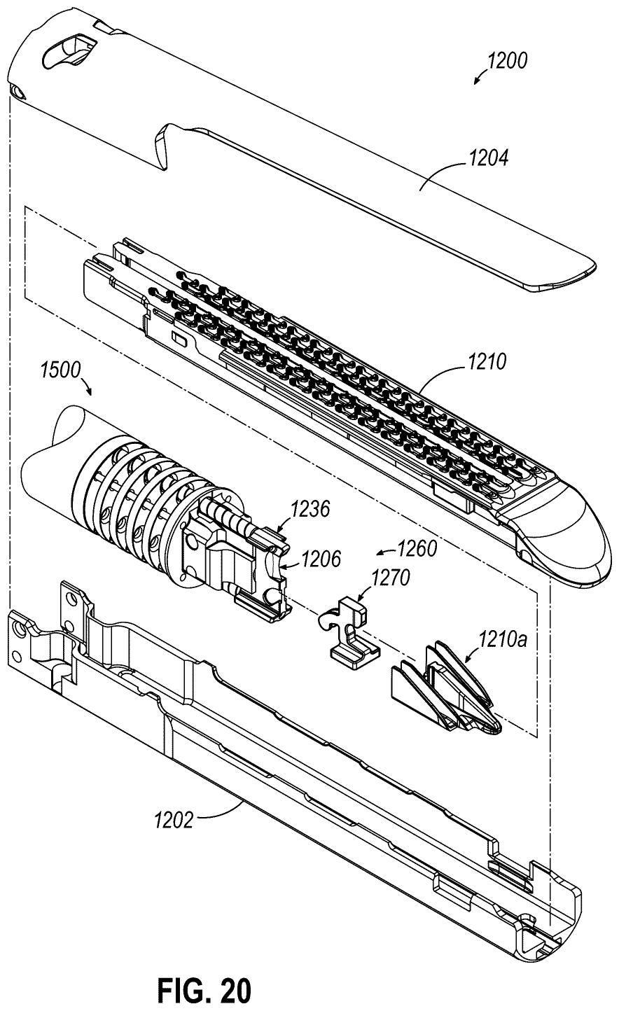

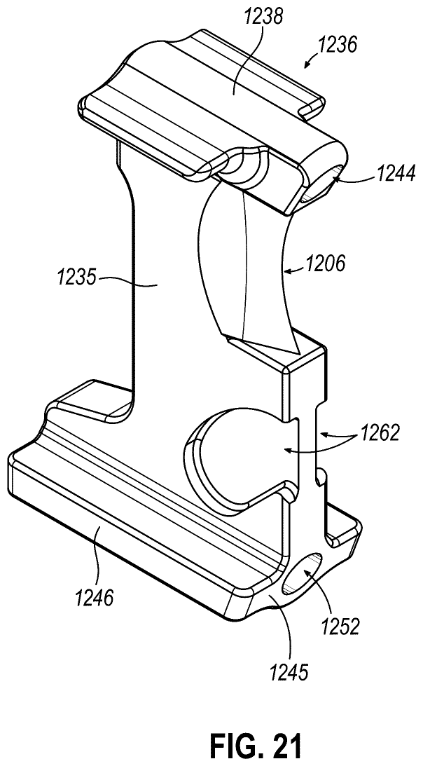

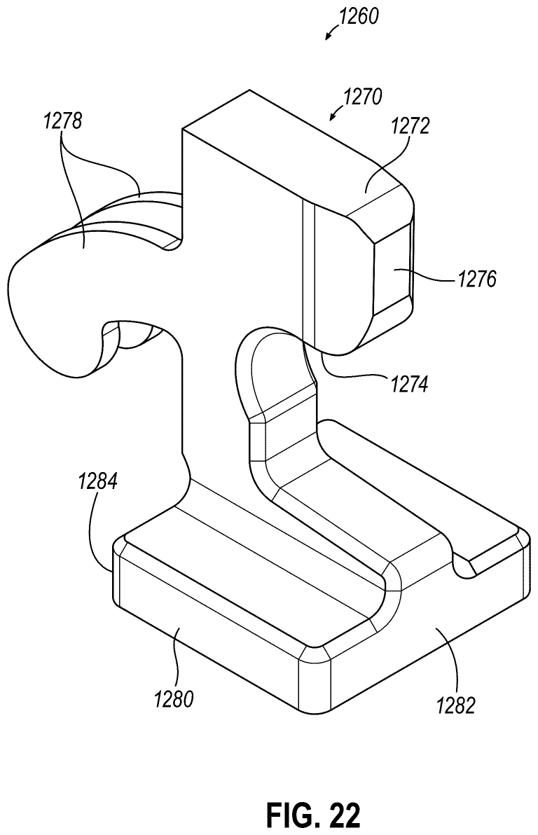

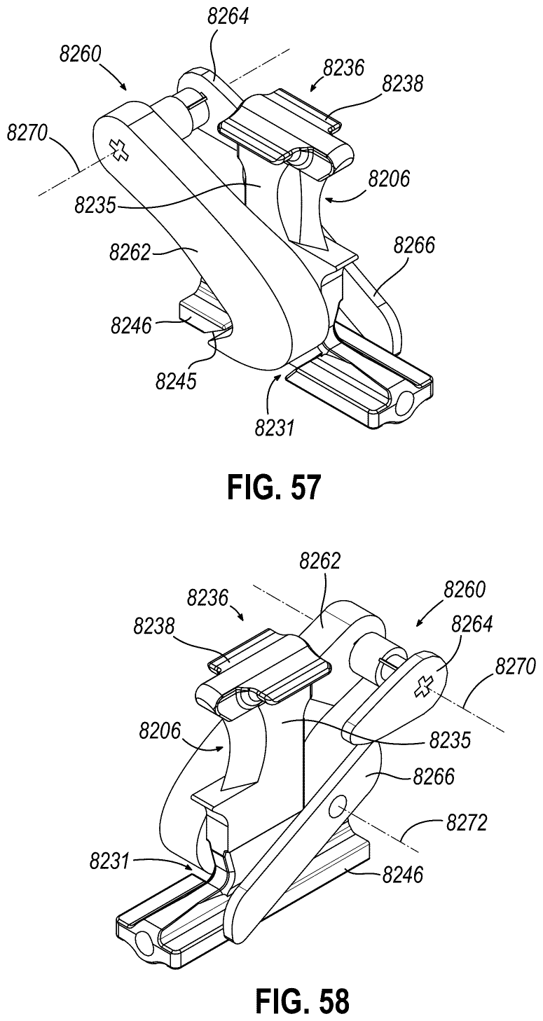

It may be desirable to inhibit firing of the surgical instrument 1000 in instances when the end effector 200 is loaded with a staple cartridge 210 that has already been fired (i.e., a “spent-cartridge” condition), and also when the end effector 200 is not loaded with any staple cartridge at all (i.e., an “absent-cartridge” condition). Attempted firings during such conditions are the result of user error and could result in unintended action on patient tissue; namely, severing the tissue without simultaneously sealing it with staples. The illustrative configurations shown and described below in connection with FIGS. 20-58B are effective to inhibit firing in each of a spent-cartridge condition and an absent-cartridge condition, and thereby protect against such unintended action on patient tissue.

A. First Illustrative End Effector with Lockout Assembly

FIGS. 20-24B show an illustrative end effector 1200 that may be readily incorporated into surgical instrument 1000 in replacement of end effector 200 described above. Therefore, end effector 1200 may be substantially similar to end effector 200 described above, with differences elaborated herein. In particular, as will be described in greater detail below, end effector 1200 includes a lockout assembly 1260 configured to inhibit firing of end effector 1200 in the spent-cartridge condition and the absent-cartridge condition.

End effector 1200 includes a cartridge jaw 1202, an anvil jaw 1204, a staple cartridge 1210, a cartridge sled 1210A, a knife 1206, and knife sled 1236; which may be substantially similar to cartridge jaw 202, anvil jaw 204, staple cartridge 210, cartridge sled 210A, knife 206, and knife sled 236 described above, respectively, with differences elaborated herein. Therefore, cartridge jaw 1202 and anvil jaw 1204 are pivotally coupled to each other and may pivot relative to each other between an open position (similar to end effector 200 shown in FIG. 8A) and a clamping position (similar to end effector 200 shown in FIGS. 8C-8D), in response to movement of knife sled 1236 in accordance with the description herein.

Knife 1206 and knife sled 1236 are suitably attached to a knife firing subsystem 1500, which is substantially similar to knife firing subsystem 500 described above. Therefore, knife firing subsystem 1500 includes first push rod 1504, a second push rod 1506, push coils 1508, 1510, and center cables 1512, 1514; which may be substantially similar to first push rod 504, second push rod 506, push coils 508, 510, and center cables 512, 514 described above.

Anvil jaw 1204 includes a longitudinally extending upper knife channel 1224 that may be substantially similar to longitudinally extending upper knife channel 224 described above. Similarly, cartridge jaw 1202 includes an elongated channel 1208 and a lower knife channel 1230 having a centrally disposed lower knife channel portion 1232 and at least one lateral lower knife channel wing 1234, which may be substantially similar to elongate channel 208, lower knife channel 230, centrally disposed lower knife channel portion 232, and lateral lower knife channel wings 234 described above, respectively, with differences elaborated below.

Knife sled 1236 includes a vertical column 1235, an upper knife tab 1238 defining an upper knife tab opening 1244, and lower knife tab 1246 defining a lower knife tab opening 1252; which may be substantially similar to vertical column 235, upper knife tab 238, upper knife tab opening 244, lower knife tab 246, and lower knife tab opening 252 described above, respectively, with differences elaborated below. Therefore, upper knife tab 1238 and lower knife tab 1246 may actuate within upper knife channel 1224 and lower knife channel 1230, respectively, as knife sled 1236 acutes relative to jaws 1202, 1204 in accordance with the description herein, thereby assisting in pivoting anvil jaw 1204 from the open position into the clamping position.

Staple cartridge 1210 slidably houses cartridge sled 1210A. Staple cartridge defines an elongated knife channel 1215 that is substantially similar to elongated knife channel 215 described above, with differences elaborated herein. Elongated channel 1208 of cartridge jaw 1202 may selectively receive a first staple cartridge 1210 such that an unfired staple cartridge 1210 may be loaded into cartridge jaw 1202 and fired in accordance with the description herein. Next, the spent staple cartridge 1210 may be removed from cartridge jaw 1202 such that a new, unfired staple cartridge staple cartridge 1210 may be loaded into cartridge jaw 1202.

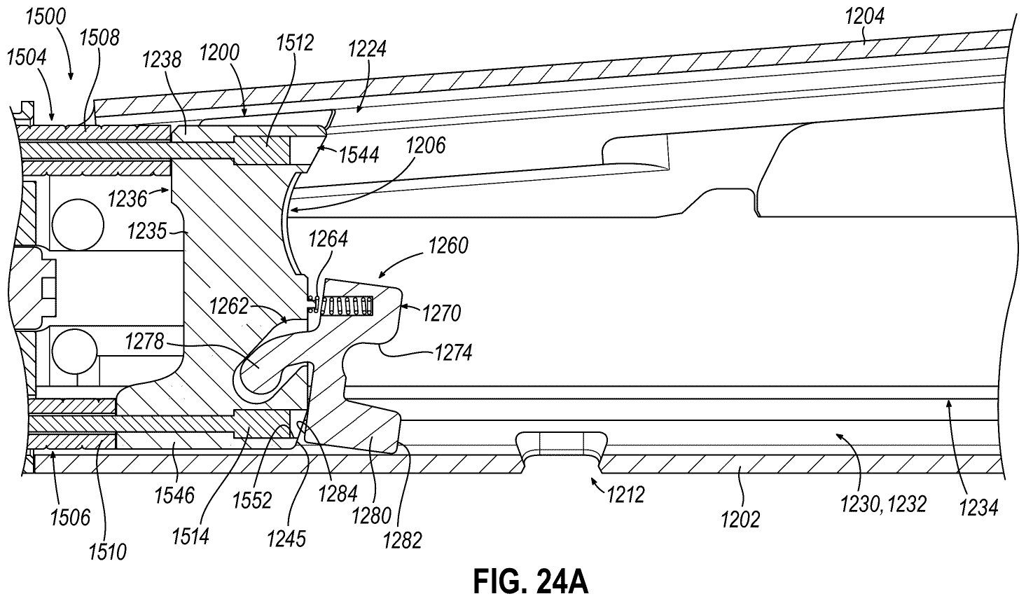

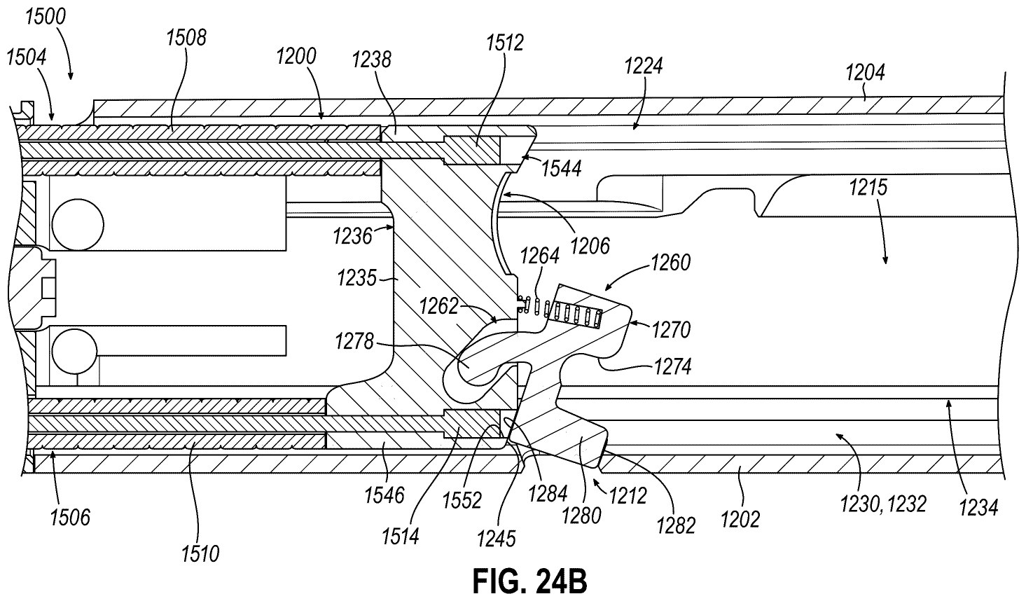

As mentioned above, end effector 1200 includes a lockout assembly 1260 configured to inhibit firing of end effector 1200 in both the spent-cartridge condition and the absent-cartridge condition. In the current illustrative example, lockout assembly 1260 includes a pivoting lockout body 1270 pivotally attached to knife sled 1236, and a biasing element, in the form of a spring 1264, that biases pivoting lockout body 1270 to pivot relative to knife sled 1236 toward a locked-out configuration (see FIG. 24B). Cartridge jaw 1202 also defines a recessed pocket 1212. Recessed pocket 1212 is located distally relative to a pre-fired cartridge sled 1210A when staple cartridge 1210 is suitably loaded within cartridge jaw 1202. While in the current example, recessed pocket 1212 is shown extending into an exterior surface of cartridge jaw 1202 (e.g., a through hole), this is merely optional. In some instances, recessed pocket 1212 has a floor such that pocket 1212 does not extend into an exterior surface of cartridge jaw 1202.

As will be described in greater detail below, when a pre-fired staple cartridge 1210 is suitably loaded into cartridge jaw 1202, pivoting lock out body 1270 is configured to engage a pre-fired cartridge sled 1210A as knife 1206 actuates distally from a proximal position, thereby bypassing recessed pocket 1212 defined by cartridge jaw 1202. As will also be described in greater detail below, if a spent-cartridge 1210 is loaded into cartridge jaw 1202 (i.e., a spent-cartridge condition), or a cartridge 1210 is absent from cartridge jaw 1202 (i.e., an absent-cartridge condition), spring 1264 will drive pivoting lockout body 1270 relative to knife sled 1236 into recessed pocket 1212, thereby inhibiting further distal actuation of knife 1206.

Spring 1264 is interposed between a proximally facing surface of pivoting lockout body 1270 and a distally facing surface of vertical column 1235 of knife sled 1236. Pivoting lockout body 1270 and spring 1264 actuate with knife 1206 and knife sled 1236 as knife 1206 is fired relative to cartridge jaw 1202 and/or staple cartridge 1210 in accordance with the description herein.

Spring 1264 biases the proximally facing surface of pivoting lockout body 1270 away from the distally facing surface of vertical column 1235 of knife sled 1236, thereby biasing pivoting lockout body 1270 toward the locked-out position (see FIG. 24B). While a linear spring 1264 is used in the current example, any other suitable biasing element may be utilized in order to bias pivoting lockout body 1270 toward the locked-out configuration as would be apparent to one skilled in the art in view of the teachings herein. For example, a torsion spring may be utilized to bias pivoting lockout body 1270. Such alternative biasing elements may have any suitable spatial relationship with other components of lockout assembly 1260 and end effector 1200 as would be apparent to one skilled in the art in view of the teachings herein.

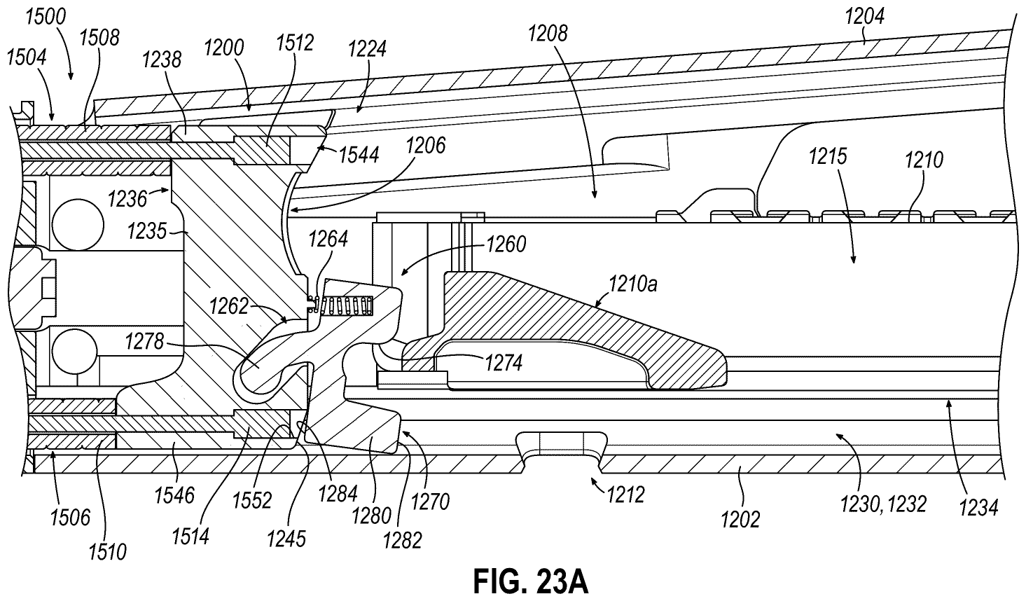

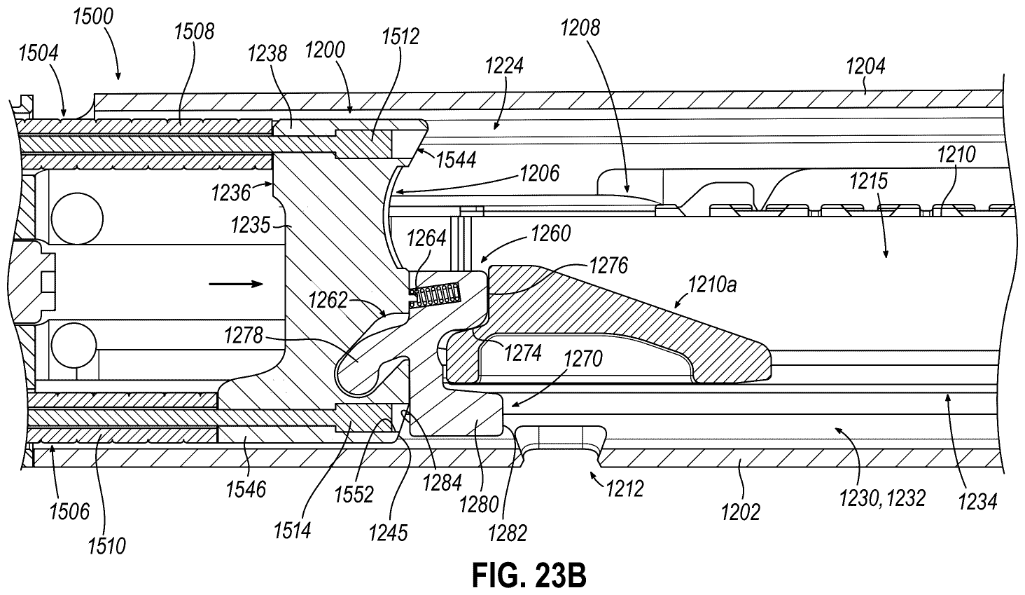

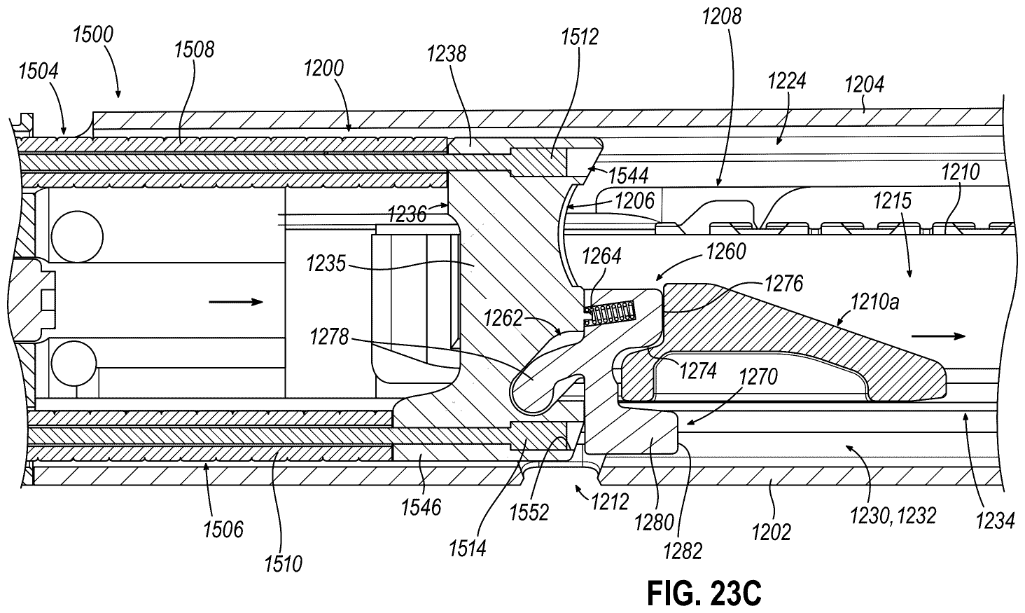

Knife sled 1236 defines a pair of curved cutouts 1262 that are on opposite lateral sides of vertical column 1235. Curved cutouts 1262 slidably house a respective proximal projection 1278 of pivoting lockout body 1270. Curved cutouts 1262 constrain movement of their respective proximal projections 1278 to at least partially guide movement of pivoting lockout body 1270 relative to knife sled 1236 as proximal projections 1278 actuate along the path defined by curve cutouts 1262. In particular, sliding engagement between curved cutouts 1262 and proximal projections 1278 guide pivoting lockout body 1270 to pivot relative to knife sled 1236 between a pre-fired configuration (see FIGS. 23A and 24A), an unlocked configuration (see FIGS. 23B-23C), and a locked-out configuration (see FIG. 24B) in accordance with the teachings herein. In the current illustrative example, the pivot axis of pivoting lockout body 1270 is located vertically between push coil 1508, 1510 of knife firing system.

While knife sled 1236 defines cutouts 1262 and pivoting lockout body 1270 includes proximal projection 1278, this is merely optional, as any other suitable structures may be utilized in order to guide pivoting lockout body 1270 relative to knife sled 1236 between the various suitable positions as would be apparent to one skilled in the art in view of the teachings herein. For instance, knife sled 1236 may include laterally presented arched projections that receive and guide complementary arched pockets defined by pivoting lockout body 1270.

Pivoting lockout body 1270 includes an upper section 1272 and a lower section 1280. Upper section 1272 of pivoting lockout body 1270 is housed within elongate channel 1208 of cartridge jaw 1202, while lower section 1280 of pivoting lockout body 1280 is housed within lower knife channel 1230 of cartridge jaw 1202. Upper section 1272 of pivoting lockout body 1270 is dimensioned to slidably fit within elongated knife channel 1215 of staple cartridge 1210. As will be described in greater detail below, when a pre-fired staple cartridge 1210 is suitably loaded, upper section 1272 is configured to suitably engage pre-fired cartridge sled 1210A in order to suitably bypass recessed pocket 1212 of cartridge jaw 1202. Additionally, as will be described in greater detail below, when either an absent-cartridge condition or a spent cartridge condition is present, lower section 1280 of pivoting lockout body 1280 is configured to engage recessed pocket 1212 of cartridge jaw 1202 to inhibit firing of knife 1206 in accordance with the description herein.

Upper section 1272 of pivoting lockout body 1270 includes a lockout bypass surface 1274, a distal sled driving surface 1276, and proximal projections 1278. As mentioned above, proximal projections 1278 are slidably contained within curved cutouts 1262 of vertical column 1235 of knife sled 1236 in order to guide pivoting lockout body 1270 to pivot between a pre-fired configuration (see FIGS. 23A and 24A), an unlocked configuration (see FIGS. 23B-23C), and a locked-out configuration (see FIG. 24B).

Lockout bypass surface 1274 includes a suitable distally presented profile that is configured to engage a complementary profile of a pre-fired cartridge sled 1210A when a pre-fired staple cartridge 1210 is suitably loaded. Engagement between lockout bypass surface 1274 and the complementary surface of a pre-fired cartridge sled 1210A inhibits spring 1264 from driving pivoting lockout body 1270 into recessed pocket 1212 as lower section 1280 of pivoting lockout body 1270 actuates over recessed pocket 1212, effectively allowing knife 1206 to bypass recessed pocket 1212.

Proximally facing surfaces of upper section 1272 suitably engages distally facing surface of vertical column 1235 of knife sled 1236 as knife 1206 is actuated distally in the unlocked configuration (see FIGS. 23B-23C), such that knife sled 1236 distally drives pivoting lockout body 1270 while knife 1206 is fired in accordance with the description herein. Further, distal sled driving surface 1276 includes a suitable distally presented profile that is configured to engage a complementary profile of cartridge sled 1210A. Engagement between the distal sled driving surface 1276 and cartridge sled 1210A is configured to drive cartridge sled 1210A distally as knife 1206 is actuated distally in accordance with the description herein. Distal actuation of cartridge sled 1210A0 is configured is drive a plurality of staples out of staple cartridge 1210, through tissue grasped between carriage 1210 and anvil 1204, and against staple forming pockets of anvil 1204 in order to suitably staple grasped tissue while knife 1206 simultaneously servers grasped tissue.

As mentioned above, in the current example, lower section 1280 of pivoting lockout body 1270 is housed within lower knife channel 1230 of cartridge jaw 1202. Lower section 1280 may include a suitable profile configured to slidably fit within lower knife channel 1230. Therefore, lower section 1280 may include a central portion that slidably fits within centrally disposed knife channel portion 1232; while also including laterally presented wings that slidably fit within lateral lower knife channel wing 1234. Lower knife channel 1230 is suitably large enough to allow lower section 1280 to pivot from the pre-fired configuration (see FIG. 24A) into the locked-out configuration (see FIG. 24B).

Lower section 1280 includes a distal lockout surface 1282 and a proximal lockout surface 1284. When pivoting lockout body 1270 is driven into the locked-out configuration in accordance with the description herein, distal lockout surface 1282 engages a section of cartridge jaw 1202 defining recessed pocket 1212 while proximal lockout surface 1284 engages a lockout engagement surface 1245 of lower knife tab 1246. Therefore, lower section 1280 of pivoting lockout body 1270 may transfer the lockout force from engagement with recessed pocket 1212 in the locked-out configuration (see FIG. 24B) onto knife sled 1236, thereby inhibiting further distal actuation of knife sled 1236 in the locked out configuration.

Engagement surfaces 1245, 1284 are angled such that as knife 1206 attempts to actuate distally in the locked-out configuration, engagement between lockout engagement surface 1245 of lower knife tab 1246 and proximal lockout surface 1284 of pivoting lockout body 1270 suitably engage each other while pivoting lockout body 1270 is pivoted into the locked-out configuration (see FIG. 24B) The lockout forces generated via engagement of pivoting lockout body 1270 and recessed pocket 1212 of cartridge jaw 1202 may be transferred onto knife sled 1236 and lower knife channel 1230 of cartridge jaw 1202 to thereby inhibit further distal actuation of knife 1206 in the locked-out configuration. A portion of the lockout forces may be transmitted axially through cartridge jaw 1202 (e.g., a proximally oriented compressive force). In some instances, the generated lockout forces may create a moment which rotates knife sled 1236 clockwise (with respect to FIG. 24B). Such a moment may encourage knife tabs (1238, 1246) to contact portions of knife channels (1224, 1230), respectively.

FIG. 23A-23C show an illustrative firing of knife 1206 while an unfired staple cartridge 1210 is suitably loaded into end effector 1200. As shown in FIG. 23A, cartridge sled 1210A is located proximally relative to recessed pocket 1212 of cartridge jaw 1202. Further, knife 1206, knife sled 1236, and pivoting lockout body 1270 are all located proximally relative to cartridge sled 1210A. Pivoting lockout body 1270 is in the pre-fired configuration. Therefore, spring 1264 biases pivoting lockout body 1270 such that a section of lower portion 1280 engages a lower surface of lower knife channel 1230. It should be understood that as knife 1206 actuates distally from the proximal position (see FIGS. 23A and 24A) toward either the unlocked configuration (see FIG. 23B) or the locked-out configuration (see FIG. 24B), lower section 1280 may ride against the lower surface of lower knife channel 1230.

While pivoting lockout body 1270 is in the pre-fired configuration (see FIGS. 23A and 24A) and the unlocked configuration (see FIG. 23B), the entirety of pivoting lockout body (1270) is located vertically between push coils 1508, 1510. While pivoting lockout body 1270 is in the locked-out configuration (see FIG. 24B), a portion of pivoting lockout body 1270 is located vertically outside the confines of push coils 1508, 1510.

During the firing process, knife 1206 actuates distally as shown in FIG. 23B. Because a pre-fired cartridge sled 1210A is present, lockout bypass surface 1274 engages cartridge sled 1210A in accordance with the description herein, thereby inhibiting spring 1264 from driving pivoting lockout body 1270 into the locked-out configuration. Additionally, engagement between lockout bypass surface 1274 may lift pivoting lockout body 1270 from the pre-fired configuration into the unlocked configuration such that distal sled driving surface 1276 suitably engages cartridge sled 1210A in accordance with the description herein. Additionally, in some instances lockout bypass surface 1275 may engage cartridge sled 1210 to further lift lower section 1280 to no longer engage the lower surface of lower knife channel 1230, as was the case while pivoting lockout body was in the pre-fried configuration.

Next, as shown in FIG. 23C, with pivoting lockout body 1270 in the unlocked configuration, knife 1206 and cartridge sled 1210A may be fired distally in accordance with the description herein, effectively bypassing the recessed pocket 1212 of cartridge jaw 1202.

FIGS. 24A-24B show an illustrative firing of knife 1206 while a staple cartridge 1210 is not present (i.e., an absent-cartridge condition). It should be understood that while FIGS. 24A-24B show an absent-cartridge condition, the same effect may take place when a spent cartridge condition is present. As mentioned above, in some instances, cartridge sled 1210A is configured to remain in the distal position after firing of end effector 1200 in accordance with the description herein. Therefore, in a spent cartridge condition, cartridge sled 1210A would be distal relative to the recessed pocket 1212, effectively rendering the same result as the absent-cartridge condition described below.

As shown in FIG. 24A, with no cartridge 1210 loaded, cartridge sled 1210A is absent and not interposed between pivoting lockout body 1270 and recessed pocket 1212. Pivoting lockout body 1270 is in the pre-fired configuration. Therefore, spring 1264 biases pivoting lockout body 1270 such that a section of lower portion 1280 engages a lower surface of lower knife channel 1230. As shown in FIG. 24B, if knife 1206 is fired in the absent-cartridge condition (or the spent-cartridge condition), spring 1264 biases pivoting lockout body 1270 downward as lockout body 1270 reaches a position adjacent to recessed pocket 1212. Distal lockout surface 1282 is effectively aligned with recessed pocket 1212 such that distal lockout surface 1282 engages recessed pocket 1212, thereby locking out knife 1206 from further distal actuation in accordance with the description herein.

B. Second Illustrative End Effector with Lockout Assembly

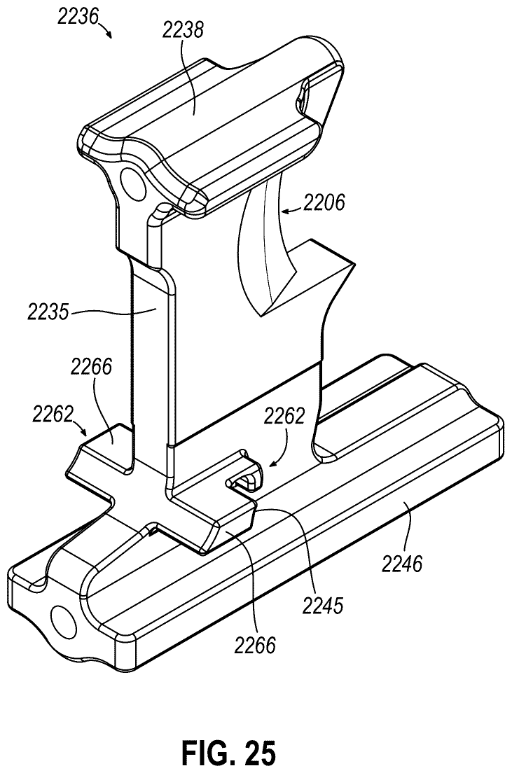

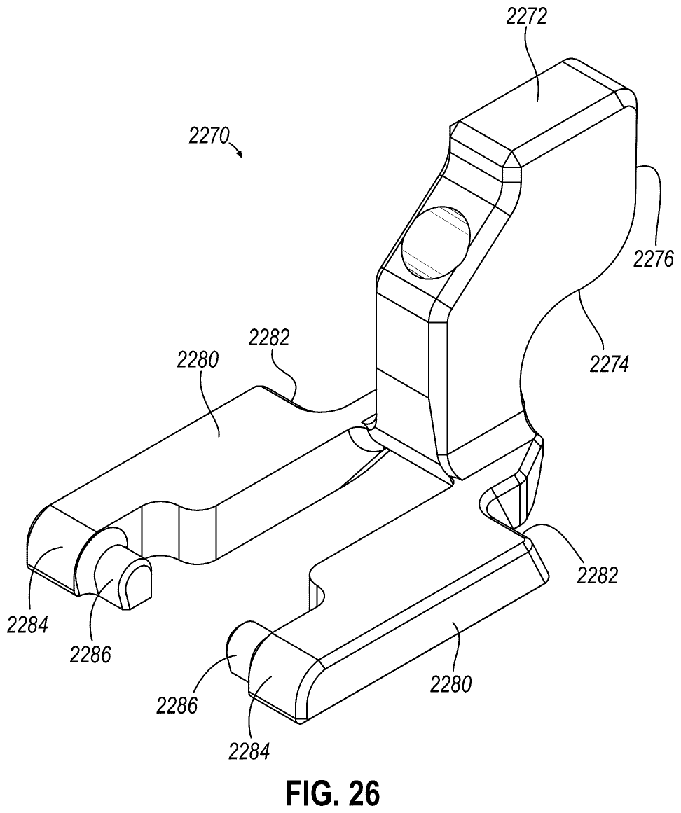

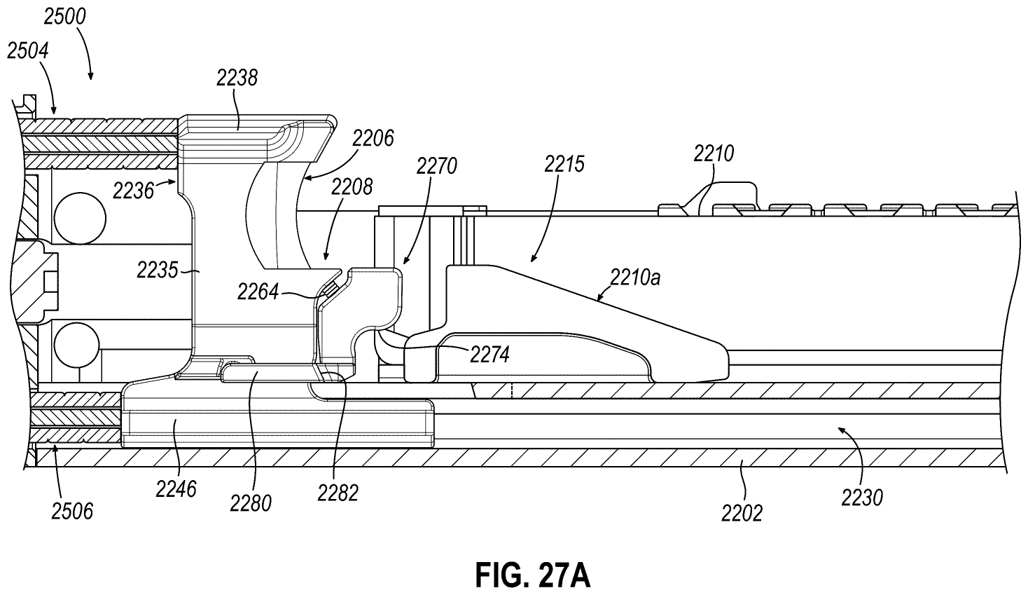

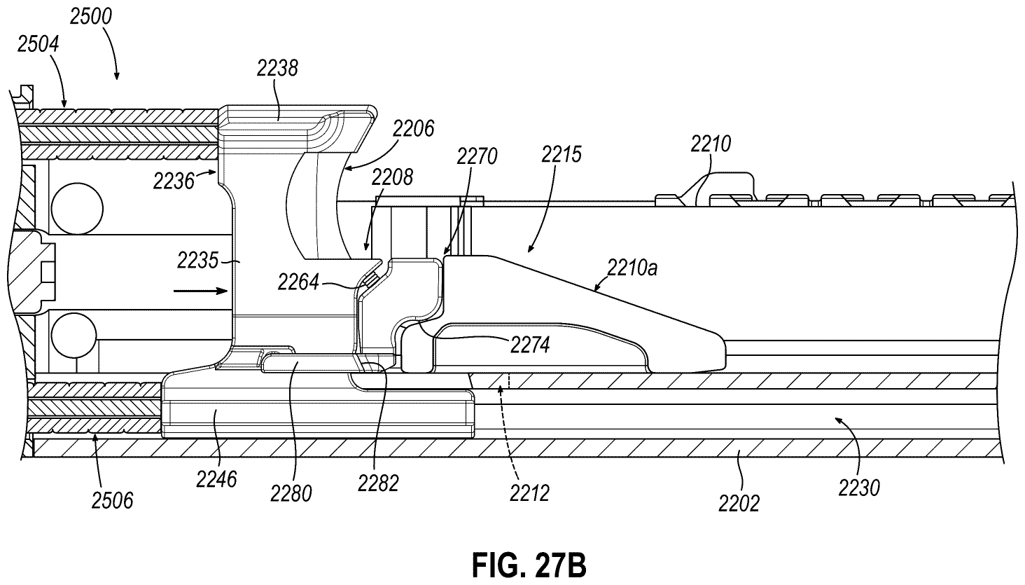

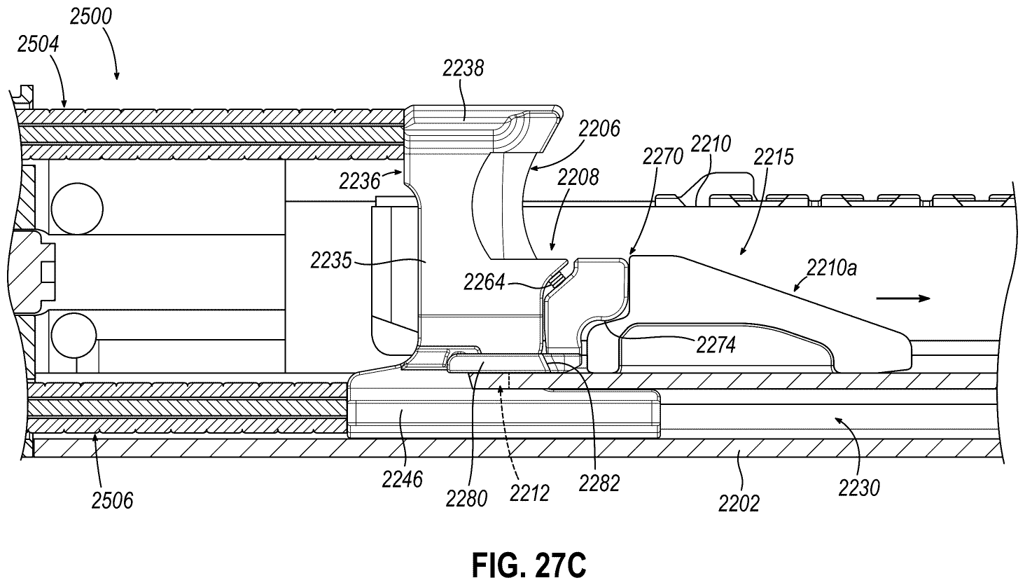

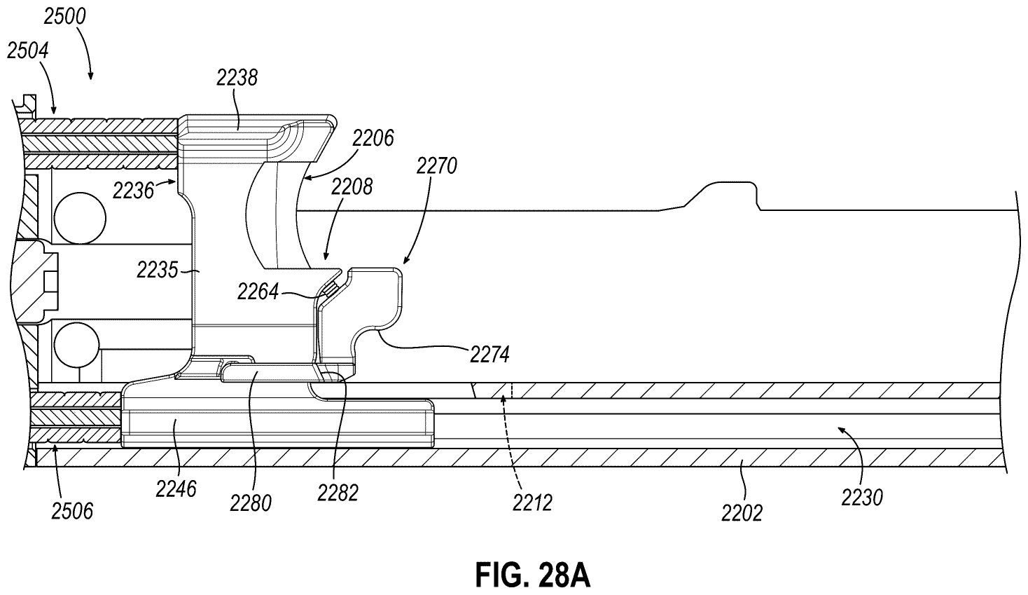

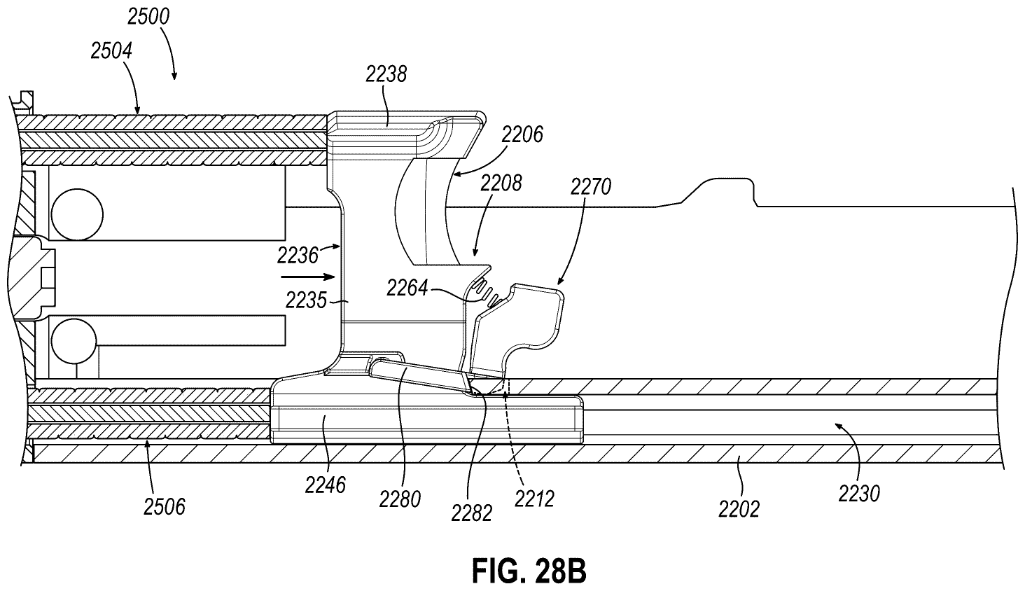

FIGS. 27A-28B show an illustrative end effector 2200 that may be readily incorporated into surgical instrument 1000 in replacement of end effector 200 described above, while FIGS. 25-26 show an illustrative knife 2206 and pivoting lockout body 2270, respectively, of end effector 2200. Therefore, end effector 2200 may be substantially similar to end effector 200, 1200 described above, with differences elaborated herein. In particular, as will be described in greater detail below, end effector 2200 includes a lockout assembly 2260 configured to inhibit firing of end effector 2200 in the spent-cartridge condition and the absent-cartridge condition.

End effector 2200 includes a cartridge jaw 2202, an anvil jaw (not shown), a staple cartridge 2210, a cartridge sled 2210A, a knife 2206, and knife sled 2236; which may be substantially similar to cartridge jaw 202, anvil jaw 204, staple cartridge 210, cartridge sled 210A, knife 206, and knife sled 236 described above, respectively, with differences elaborated herein. Therefore, cartridge jaw 2202 and anvil jaw 2204 are pivotally coupled to each other and may pivot relative to each other between an open position (similar to end effector 200 shown in FIG. 8A) and a clamping position (similar to end effector 200 shown in FIGS. 8C-8D), in response to movement of knife sled 2236 in accordance with the description herein.

Knife 2206 and knife sled 2236 are suitably coupled to first and second push rods 2504, 2506 of knife firing subsystem 2500; which is substantially similar to knife firing subsystem 500, 1500 described above.

Anvil jaw (not shown) includes a longitudinally extending upper knife channel (not shown) that may be substantially similar to longitudinally extending upper knife channel 224 described above. It should be understood that anvil jaw is omitted for purposes of clarity. Similarly, cartridge jaw 2202 includes an elongated channel 2208 and a lower knife channel 2230, which may be substantially similar to elongate channel 208, and lower knife channel 230 described above, respectively, with differences elaborated below.

Knife sled 2236 includes a vertical column 2235, an upper knife tab 2238, and lower knife tab 2246; which may be substantially similar to vertical column 235, upper knife tab 238, and lower knife tab 246 described above, respectively, with differences elaborated below. Therefore, upper knife tab 2238 and lower knife tab 2246 may actuate within upper knife channel 2224 and lower knife channel 2230, respectively, as knife sled 2236 acutes relative to jaws 2202, 2204 in accordance with the description herein, thereby assisting in pivoting anvil jaw 2204 from the open position into the clamping position.

Staple cartridge 2210 slidably houses cartridge sled 2210A. Staple cartridge defines an elongated knife channel 2215 that is substantially similar to elongated knife channel 215 described above, with differences elaborated herein. Elongated channel 2208 of cartridge jaw 2202 may selectively receive a first staple cartridge 2210 such that an unfired staple cartridge 2210 may be loaded into cartridge jaw 2202 and fired in accordance with the description herein. Next, the spent staple cartridge 2210 may be removed from cartridge jaw 2202 such that a new, unfired staple cartridge staple cartridge 2210 may be loaded into cartridge jaw 2202.

As mentioned above, end effector 2200 includes a lockout assembly 2260 configured to inhibit firing of end effector 2200 in both the spent-cartridge condition and the absent-cartridge condition. In the current illustrative example, lockout assembly 2260 includes a pivoting lockout body 2270 pivotally attached to knife sled 2236, and a biasing element, in the form of a spring 2264, that biases pivoting lockout body 2270 to pivot relative to knife sled 2236 toward a locked-out configuration (see FIG. 28B). Cartridge jaw 2202 also defines a recessed pocket 2212. Recessed pocket 2212 is located distally relative to a pre-fired cartridge sled 2210A when staple cartridge 2210 is suitably loaded within cartridge jaw 2202. While in the current example, recessed pocket 2212 is shown extending between elongated channel 2208 and lower knife channel 2230 of cartridge jaw 2202 (e.g., a through hole), this is merely optional. In some instances, recessed pocket 2212 has a floor such that pocket 2212 does not extend into lower knife channel 2230 of cartridge jaw 1202.

As will be described in greater detail below, when a pre-fired staple cartridge 2210 is suitably loaded into cartridge jaw 2202, pivoting lockout body 2270 is configured to engage a pre-fired cartridge sled 2210A as knife 2206 actuates distally from a proximal position, thereby bypassing recessed pocket 2212 defined by cartridge jaw 2202. As will also be described in greater detail below, if a spent-cartridge 2210 is loaded into cartridge jaw 2202 (i.e., a spent-cartridge condition), or a cartridge 2210 is absent from cartridge jaw 2202 (i.e., an absent-cartridge condition), spring 2264 will drive pivoting lockout body 2270 relative to knife sled 2236 into recessed pocket 2212, thereby inhibiting further distal actuation of knife 2206.

Spring 2264 is substantially similar to spring 1264 described above. Therefore, spring 2264 biases pivoting lockout body 2270 toward a lockout-out position (see FIG. 28B).

Knife sled 2236 includes a pair of lateral wings 2266 extending laterally away from vertical column 2235, which are located within elongated channel 2208. Wings 2266 includes a pivot coupling 2262 and a lockout engagement surface 2245. As will be described in greater detail below, lockout engagement surface 2245 is configured to receive a lockout force from pivot lockout body 2270 in the locked-out configuration. Pivot couplings 2262 are configured to pivotally receive pivot points 2286 of pivoting lockout body 2270, thereby allowing pivoting lockout body 2270 to pivot relative to knife sled 2236 between a pre-fired configuration (see FIGS. 27A and 28A), an unlocked configuration (see FIGS. 27B-27C), and a locked-out configuration (see FIG. 28B) in accordance with the teachings herein. Pivoting lockout body 2270 may be pivotally coupled to knife sled 2236 via any other suitable structures as would be apparent to one skilled in the art in view of the teachings herein. In the current illustrative example, the pivot axis of pivoting lockout body 2270 is located vertically between push rods 2504, 2406 (including push coils) of knife firing system 2500.

Pivoting lockout body 2270 includes an upper section 2272 and a lower section 2280. In the current example, both upper section 2272 and lower section 2280 are housed within elongate channel 2208 of cartridge jaw 2202. Upper section 2272 of pivoting lockout body 2270 is dimensioned to slidably fit within elongated knife channel 2215 of staple cartridge 2210, while lower section 2272 is configured to slidably fit within a lower pocket defined by staple cartridge 2210 that is in communication with elongated knife channel 2215. As will be described in greater detail below, when a pre-fired staple cartridge 2210 is suitably loaded, upper section 2272 is configured to suitably engage pre-fired cartridge sled 2210A in order to suitably bypass recessed pocket 2212 of cartridge jaw 2202. Additionally, as will be described in greater detail below, when either an absent-cartridge condition or a spent cartridge condition is present, lower section 2280 of pivoting lockout body 2280 is configured to engage recessed pocket 2212 of cartridge jaw 2202 to inhibit firing of knife 2206 in accordance with the description herein.

Upper section 2272 of pivoting lockout body 2270 is sustainably similar to upper section 1272 described above, with differences elaborated herein. Therefore, upper section 2272 includes a lockout bypass surface 2274 and a distal sled driving surface 2276, which are substantially similar to lockout bypass surface 1274 and distal sled driving surface 1276 described above. Upper section 2272 of the current example does not include any proximal projections to pivotally couple lockout body 2270 with knife sled 2236. Proximally facing surfaces of upper section 2272 suitably engages distally facing surface of vertical column 2235 of knife sled 2236 as knife 2206 is actuated distally in the unlocked configuration (see FIGS. 27B-27C), such that knife sled 2236 distally drives pivoting lockout body 2270 while knife 2206 is fired in accordance with the description herein.

Lower section 2280 includes a pair of arms that are laterally offset from upper section 2272 and extend proximally therefrom. Each arm of lower section 2280 includes pivot points 2286 as described above. Additionally, each arm of lower section 2280 includes a distal lockout surface 2282 and a proximal lockout surface 2284. When pivoting lockout body 2270 is driven into the locked-out configuration in accordance with the description herein, distal lockout surface 2282 engages a section of cartridge jaw 2202 defining recessed pocket 2212 while proximal lockout surface 2284 engages a lockout engagement surface 2245 of lateral wings 2266 attached to vertical column 2235. Therefore, lower section 2280 of pivoting lockout body 2270 may transfer the lockout force from engagement with recessed pocket 2212 in the locked-out configuration (see FIG. 28B) onto knife sled 2236, thereby inhibiting further distal actuation of knife sled 2236 in the locked out configuration.

Engagement surfaces 2245, 2284 are angled such that as knife 2206 attempts to actuate distally in the locked-out configuration, engagement between lockout engagement surface 2245 of lateral wing 2266 and proximal lockout surface 2284 of pivoting lockout body 2270 drives knife sled 2236 upwards. Such upward action of knife sled 2236 may promote engagement of lower knife tab 2246 with a surface of cartridge jaw 2202 defining lower knife channel 2230. Therefore, the lockout forces generated via engagement of pivoting lockout body 2270 and recessed pocket 2212 of cartridge jaw 2202 may be transferred onto knife sled 2236 and lower knife channel 2230 of cartridge jaw 2202 to thereby inhibit further distal actuation of knife 2206 in the locked-out configuration.

FIG. 27A-27C show an illustrative firing of knife 2206 while an unfired staple cartridge 2210 is suitably loaded into end effector 2200; which may be substantially similar to the illustrative firing of end effector 1200 with an unfired staple cartridge 1210 suitably loaded thereon. Therefore, as knife 2206 is actuated distally from the position shown in FIG. 27A to the position shown in FIG. 27B, lockout bypass surface 2274 engages cartridge sled 2210A in accordance with the description herein, thereby inhibiting spring 2264 from driving pivoting lockout body 2270 into the locked-out configuration. Additionally, distal sled driving surface 2276 suitably engages cartridge sled 2210A in accordance with the description herein. Next, as shown in FIG. 27C, with pivoting lockout body 2270 in the unlocked configuration, knife 2206 and cartridge sled 2210A may be fired distally in accordance with the description herein, effectively bypassing the recessed pocket 2212 of cartridge jaw 2202.

FIGS. 28A-28B show an illustrative firing of knife 2206 while a staple cartridge 2210 is not present (i.e., an absent-cartridge condition). It should be understood that while FIGS. 28A-28B show an absent-cartridge condition, the same effect may take place when a spent cartridge condition is present. As mentioned above, in a spent cartridge condition, cartridge sled 2210A would be distal relative to the recessed pocket 2212, effectively rendering the same result as the absent-cartridge condition described below.

As shown in FIG. 28A, with no cartridge 2210 loaded, cartridge sled 2210A is absent and not interposed between pivoting lockout body 2270 and recessed pocket 2212. Pivoting lockout body 2270 is in the pre-fired configuration. Therefore, spring 2264 biases pivoting lockout body 2270 such that a section of lower portion 2280 engages a lower surface of elongated channel 2208. As shown in FIG. 28B, if knife 2206 is fired in the absent-cartridge condition (or the spent-cartridge condition), spring 2264 biases pivoting lockout body 2270 downward as lockout body 2270 reaches a position adjacent to recessed pocket 2212. Distal lockout surface 2282 is effectively aligned with recessed pocket 2212 such that distal lockout surface 2282 engages recessed pocket 2212, thereby locking out knife 2206 from further distal actuation in accordance with the description herein.

C. Third Illustrative End Effector with Lockout Assembly

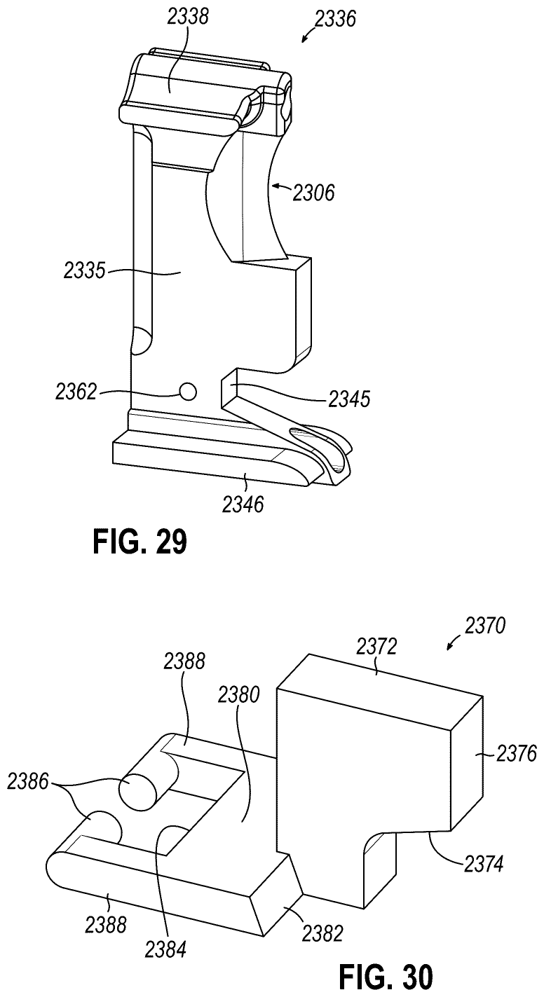

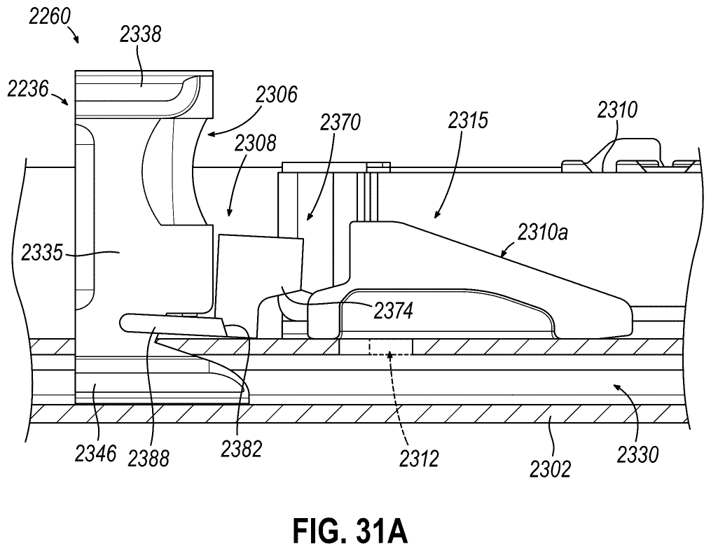

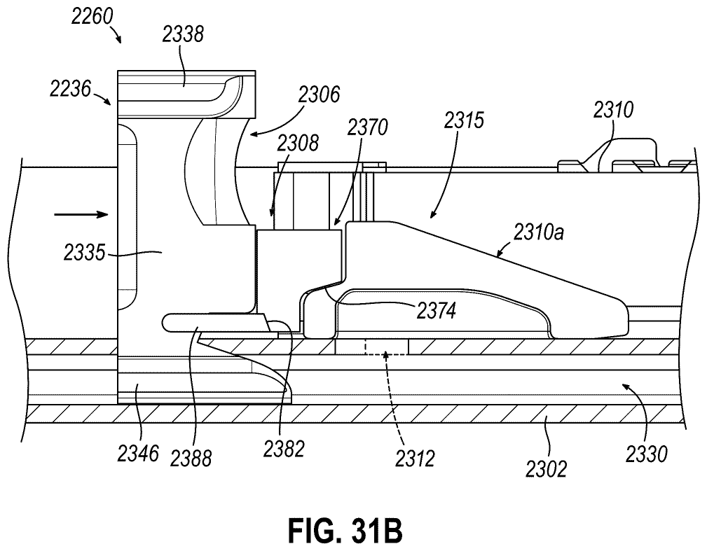

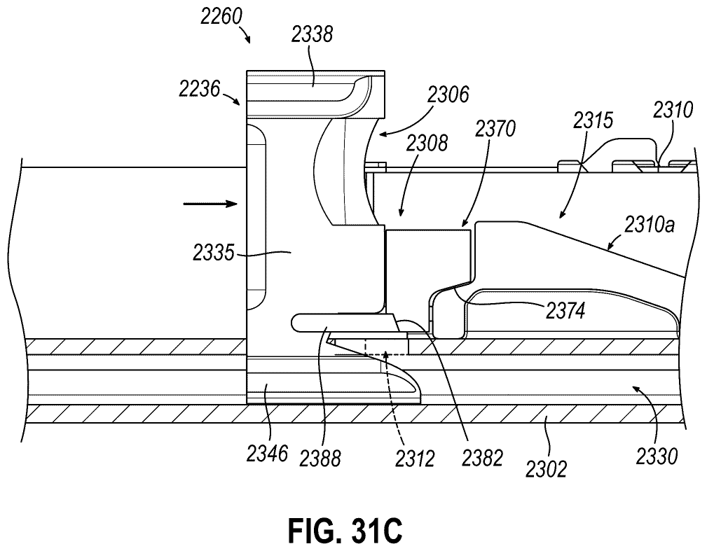

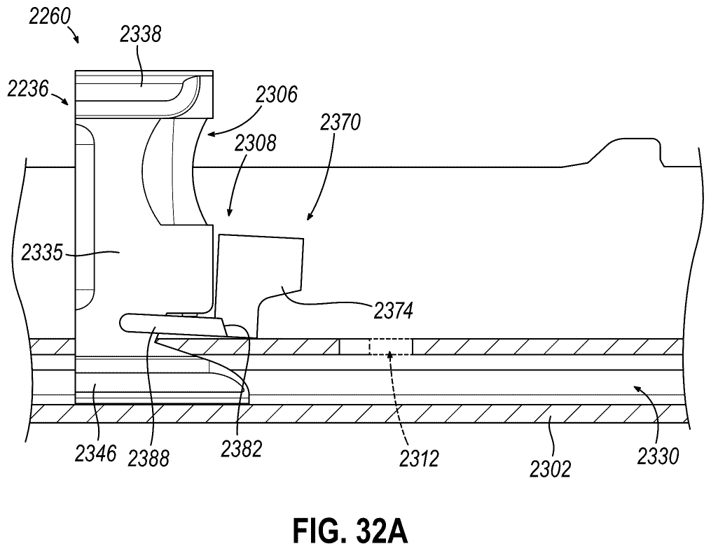

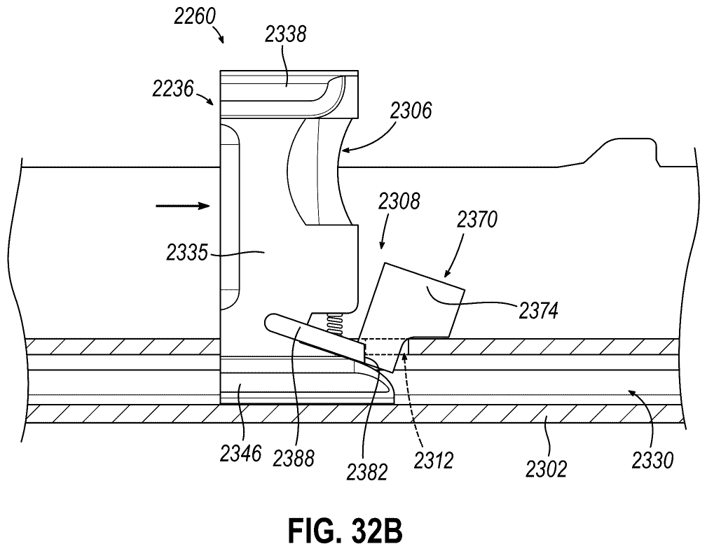

FIGS. 31A-32B show an illustrative end effector 2300 that may be readily incorporated into surgical instrument 1000 in replacement of end effector 200 described above, while FIGS. 29-30 show an illustrative knife 2306 and pivoting lockout body 2370, respectively, of end effector 2300. Therefore, end effector 2300 may be substantially similar to end effector 200, 1200, 2200 described above, with differences elaborated herein. In particular, as will be described in greater detail below, end effector 2300 includes a lockout assembly 2360 configured to inhibit firing of end effector 2300 in the spent-cartridge condition and the absent-cartridge condition.

End effector 2300 includes a cartridge jaw 2302, an anvil jaw 2304, a staple cartridge 2310, a cartridge sled 2310A, a knife 2306, and knife sled 2336; which may be substantially similar to cartridge jaw 202, anvil jaw 204, staple cartridge 210, cartridge sled 210A, knife 206, and knife sled 236 described above, respectively, with differences elaborated herein. Therefore, cartridge jaw 2302 and anvil jaw 2304 are pivotally coupled to each other and may pivot relative to each other between an open position (similar to end effector 200 shown in FIG. 8A) and a clamping position (similar to end effector 200 shown in FIGS. 8C-8D), in response to movement of knife sled 2336 in accordance with the description herein.

While not shown, knife 2306 and knife sled 2336 are coupled to a first and second push rods (not shown) of a knife firing subsystem (not shown), which are substantially similar to push rods 504, 506 and knife firing system 500 described above. Therefore, it should be understood that a first push rod (not shown) extending proximally from upper knife tab 2338 while a second push rod (not shown) extends proximally from lower knife tab 2346.

Anvil jaw (not shown) includes a longitudinally extending upper knife channel (not shown) that may be substantially similar to longitudinally extending upper knife channel 224 described above. It should be understood that anvil jaw is omitted for purposes of clarity. Similarly, cartridge jaw 2302 includes an elongated channel 2308 and a lower knife channel 2330, which may be substantially similar to elongate channel 208, and lower knife channel 230 described above, respectively, with differences elaborated below.

Knife sled 2336 includes a vertical column 2335, an upper knife tab 2338, and lower knife tab 2346; which may be substantially similar to vertical column 235, upper knife tab 238, and lower knife tab 246 described above, respectively, with differences elaborated below. Therefore, upper knife tab 2338 and lower knife tab 2346 may actuate within upper knife channel 2324 and lower knife channel 2330, respectively, as knife sled 2336 acutes relative to jaws 2302, 2304 in accordance with the description herein, thereby assisting in pivoting anvil jaw 2304 from the open position into the clamping position.

Staple cartridge 2310 slidably houses cartridge sled 2310A. Staple cartridge defines an elongated knife channel 2315 that is substantially similar to elongated knife channel 215 described above, with differences elaborated herein. Elongated channel 2308 of cartridge jaw 2302 may selectively receive a first staple cartridge 2310 such that an unfired staple cartridge 2310 may be loaded into cartridge jaw 2302 and fired in accordance with the description herein. Next, the spent staple cartridge 2310 may be removed from cartridge jaw 2302 such that a new, unfired staple cartridge staple cartridge 2310 may be loaded into cartridge jaw 2302.

As mentioned above, end effector 2300 includes a lockout assembly 2360 configured to inhibit firing of end effector 2300 in both the spent-cartridge condition and the absent-cartridge condition. In the current illustrative example, lockout assembly 2360 includes a pivoting lockout body 2370 pivotally attached to knife sled 2336, and a biasing element, in the form of a spring 2364, that biases pivoting lockout body 2370 to pivot relative to knife sled 2336 toward a locked-out configuration (see FIG. 32B). Cartridge jaw 2302 also defines a recessed pocket 2312. Recessed pocket 2312 is located distally relative to a pre-fired cartridge sled 2310A when staple cartridge 2310 is suitably loaded within cartridge jaw 2302. While in the current example, recessed pocket 2312 is shown extending between elongated channel 2308 and lower knife channel 2330 of cartridge jaw 2302 (e.g., a through hole), this is merely optional. In some instances, recessed pocket 2312 has a floor such that pocket 2312 does not extend into lower knife channel 2330 of cartridge jaw 1302.

As will be described in greater detail below, when a pre-fired staple cartridge 2310 is suitably loaded into cartridge jaw 2302, pivoting lock out body 2370 is configured to engage a pre-fired cartridge sled 2310A as knife 2306 actuates distally from a proximal position, thereby bypassing recessed pocket 2312 defined by cartridge jaw 2302. As will also be described in greater detail below, if a spent-cartridge 2310 is loaded into cartridge jaw 2302 (i.e., a spent-cartridge condition), or a cartridge 2310 is absent from cartridge jaw 2302 (i.e., an absent-cartridge condition), spring 2364 will drive pivoting lockout body 2370 relative to knife sled 2336 into recessed pocket 2312, thereby inhibiting further distal actuation of knife 2306.

Spring 2364 is substantially similar to spring 1264 described above. Therefore, spring 2364 biases pivoting lockout body 2370 toward a lockout-out position (see FIG. 32B).

Knife sled 2336 includes a pair of pivot locations 2362 located on laterally opposing sides of vertical column 2335. Knife sled 2336 also includes a lockout engagement surface 2345 located on a distally facing surface of vertical column 2335. As will be described in greater detail below, lockout engagement surface 2345 is configured to receive a lockout force from pivot lockout body 2370 in the locked out configuration. Lockout engagement surface 2345 of the current example is centrally located on vertical column 2335, as compared to lockout engagement surfaces 2245 of the previous embodiment, which were laterally offset from vertical column 22344 via lateral wings 2266. Pivot couplings 2362 are configured to pivotally receive pivot points 2386 of pivoting lockout body 2370, thereby allowing pivoting lockout body 2370 to pivot relative to knife sled 2336 between a pre-fired configuration (see FIGS. 31A and 31A), an unlocked configuration (see FIGS. 31B-31C), and a locked-out configuration (see FIG. 232B) in accordance with the teachings herein. Pivoting lockout body 2370 may be pivotally coupled to knife sled 2336 via any other suitable structures as would be apparent to one skilled in the art in view of the teachings herein.

Pivoting lockout body 2370 includes an upper section 2372 and a lower section 2380. In the current example, both upper section 2372 and lower section 2380 are housed within elongate channel 2308 of cartridge jaw 2302. Upper section 2372 of pivoting lockout body 2370 is dimensioned to slidably fit within elongated knife channel 2315 of staple cartridge 2310, while lower section 2372 is configured to slidably fit within a lower pocket defined by staple cartridge 2310 that is in communication with elongated knife channel 2315. As will be described in greater detail below, when a pre-fired staple cartridge 2310 is suitably loaded, upper section 2372 is configured to suitably engage pre-fired cartridge sled 2310A in order to suitably bypass recessed pocket 2312 of cartridge jaw 2302. Additionally, as will be described in greater detail below, when either an absent-cartridge condition or a spent cartridge condition is present, lower section 2380 of pivoting lockout body 2370 is configured to engage recessed pocket 2312 of cartridge jaw 2302 to inhibit firing of knife 2306 in accordance with the description herein.

Upper section 2372 of pivoting lockout body 2370 is sustainably similar to upper section 2272 described above, with differences elaborated herein. Therefore, upper section 2372 includes a lockout bypass surface 2374 and a distal sled driving surface 2376, which are substantially similar to lockout bypass surface 2274 and distal sled driving surface 2276 described above. Upper section 2372 of the current example does not include any proximal projections to pivotally couple lockout body 2370 with knife sled 2336. Proximally facing surfaces of upper section 2372 suitably engages distally facing surface of vertical column 2335 of knife sled 2336 as knife 2306 is actuated distally in the unlocked configuration (see FIGS. 31B-31C), such that knife sled 2336 distally drives pivoting lockout body 2370 while knife 2306 is fired in accordance with the description herein.

Lower section 2380 includes a pair of arms 2388 that are laterally offset from a base of lower section 2380 and extend proximally therefrom. Each arm 2388 of lower section 2380 includes pivot points 2386 as described above. The pivot axis of pivoting lockout body 2370 is located vertically between push rods (including push coils) of knife firing system.

Additionally, the base of lower section 2380 includes a pair of distal lockout surfaces 2382 and a centrally located proximal lockout surface 2484. When pivoting lockout body 2370 is driven into the locked-out configuration in accordance with the description herein, distal lockout surface 2382 engages a section of cartridge jaw 2302 defining recessed pocket 2312 while proximal lockout surface 2384 engages a lockout engagement surface 2345 of disposed on the distally facing surface of vertical column 2335. Therefore, lower section 2380 of pivoting lockout body 2370 may transfer the lockout force from engagement with recessed pocket 2312 in the locked-out configuration (see FIG. 32B) onto knife sled 2336, thereby inhibiting further distal actuation of knife sled 2336 in the locked-out configuration.

Engagement surfaces 2345, 2384 are angled such that as knife 2306 attempts to actuate distally in the locked-out configuration, engagement between lockout engagement surface 2345 of vertical column 2335 and proximal lockout surface 2384 of pivoting lockout body 2370 drives knife sled 2336 upwards. Such upward action of knife sled 2336 may promote engagement of lower knife tab 2346 with a surface of cartridge jaw 2302 defining lower knife channel 2330. Therefore, the lockout forces generated via engagement of pivoting lockout body 2370 and recessed pocket 2312 of cartridge jaw 2302 may be transferred onto knife sled 2336 and lower knife channel 2330 of cartridge jaw 2302 to thereby inhibit further distal actuation of knife 2306 in the locked-out configuration.

FIG. 31A-31C show an illustrative firing of knife 2306 while an unfired staple cartridge 2310 is suitably loaded into end effector 2300; which may be substantially similar to the illustrative firing of end effector 1200 with an unfired staple cartridge 1210 suitably loaded thereon. Therefore, as knife 2306 is actuated distally from the position shown in FIG. 31A to the position shown in FIG. 31B, lockout bypass surface 2374 engages cartridge sled 2310A in accordance with the description herein, thereby inhibiting spring 2364 from driving pivoting lockout body 2370 into the locked-out configuration. Additionally, distal sled driving surface 2376 suitably engages cartridge sled 2310A in accordance with the description herein. Next, as shown in FIG. 31C, with pivoting lockout body 2370 in the unlocked configuration, knife 2306 and cartridge sled 2310A may be fired distally in accordance with the description herein, effectively bypassing the recessed pocket 2312 of cartridge jaw 2302.

FIGS. 32A-32B show an illustrative firing of knife 2306 while a staple cartridge 2310 is not present (i.e., an absent-cartridge condition). It should be understood that while FIGS. 32A-32B show an absent-cartridge condition, the same effect may take place when a spent cartridge condition is present. As mentioned above, in a spent cartridge condition, cartridge sled 2310A would be distal relative to the recessed pocket 2312, effectively rendering the same result as the absent-cartridge condition described below.

As shown in FIG. 32A, with no cartridge 2310 loaded, cartridge sled 2310A is absent and not interposed between pivoting lockout body 2370 and recessed pocket 2312. Pivoting lockout body 2370 is in the pre-fired configuration. Therefore, spring 2364 biases pivoting lockout body 2370 such that a section of lower portion 2380 engages a lower surface of elongated channel 2308. As shown in FIG. 32B, if knife 2306 is fired in the absent-cartridge condition (or the spent-cartridge condition), spring 2364 biases pivoting lockout body 2370 downward as lockout body 2370 reaches a position adjacent to recessed pocket 2312. Distal lockout surface 2382 is effectively aligned with recessed pocket 2312 such that distal lockout surface 2382 engages recessed pocket 2312, thereby locking out knife 2306 from further distal actuation in accordance with the description herein.

D. Fourth Illustrative End Effector with Lockout Assembly

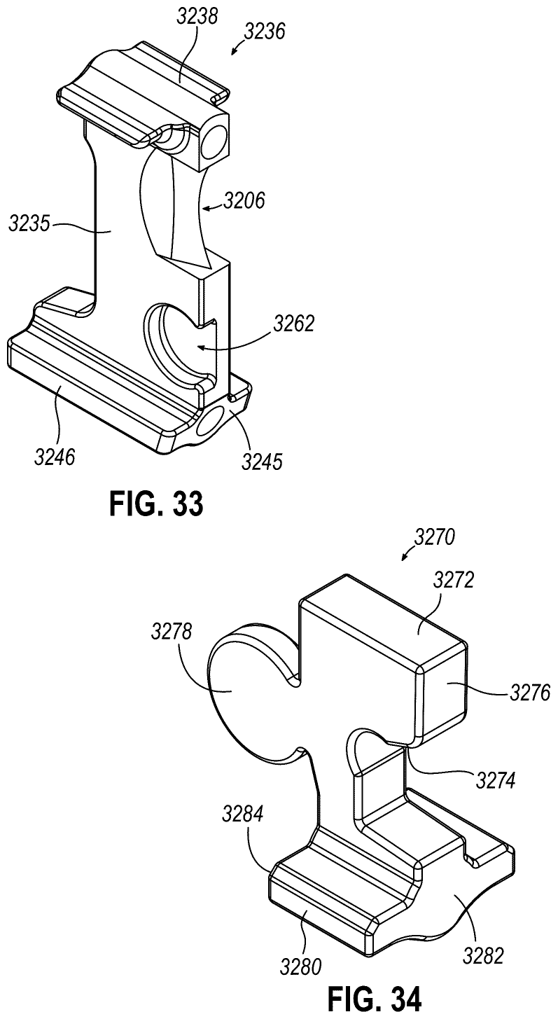

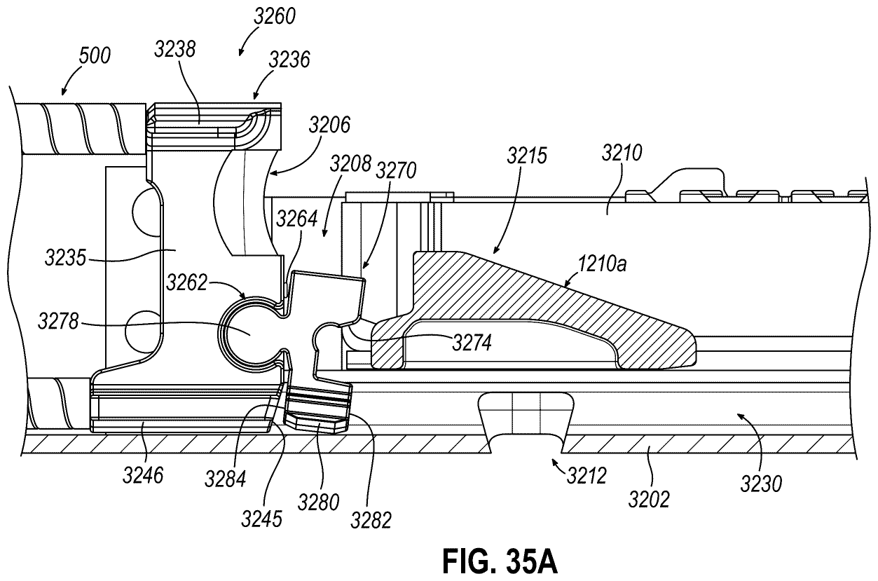

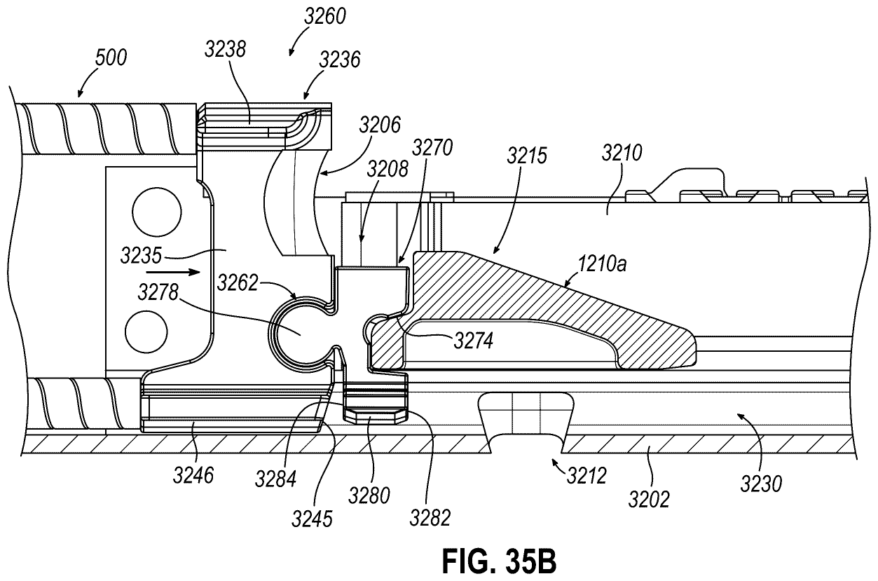

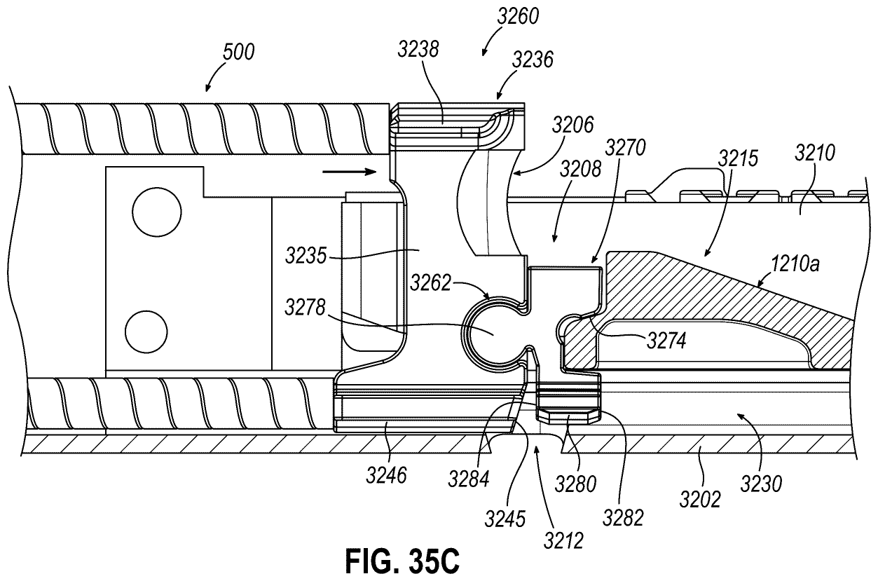

FIGS. 35A-35B show an illustrative end effector 3200 that may be readily incorporated into surgical instrument 1000 in replacement of end effector 200 described above, while FIGS. 33-34 show an illustrative knife 3206 and pivoting lockout body 3270, respectively, of end effector 3200. Therefore, end effector 3200 may be substantially similar to end effector 200, 1200, 2200, 2300 described above, with differences elaborated herein. In particular, as will be described in greater detail below, end effector 3200 includes a lockout assembly 3260 configured to inhibit firing of end effector 3200 in the spent-cartridge condition and the absent-cartridge condition.

End effector 3200 includes a cartridge jaw 3202, an anvil jaw 3204, a staple cartridge 3210, a cartridge sled 3210A, a knife 3206, and knife sled 3236; which may be substantially similar to cartridge jaw 202, anvil jaw 204, staple cartridge 210, cartridge sled 210A, knife 206, and knife sled 236 described above, respectively, with differences elaborated herein. Therefore, cartridge jaw 3202 and anvil jaw 3204 are pivotally coupled to each other and may pivot relative to each other between an open position (similar to end effector 200 shown in FIG. 8A) and a clamping position (similar to end effector 200 shown in FIGS. 8C-8D), in response to movement of knife sled 3236 in accordance with the description herein.

Knife 3206 and knife sled 3236 are suitably attached to knife firing subsystem 500 such that push coils 508, 510 extend proximally from upper knife tab 3238 and lower knife tab 3246, respectively.

Anvil jaw (not shown) includes a longitudinally extending upper knife channel (not shown) that may be substantially similar to longitudinally extending upper knife channel 224 described above. It should be understood that anvil jaw is omitted for purposes of clarity. Similarly, cartridge jaw 3202 includes an elongated channel 3208 and a lower knife channel 3230, which may be substantially similar to elongate channel 208, and lower knife channel 230 described above, respectively, with differences elaborated below.

Knife sled 3236 includes a vertical column 3235, an upper knife tab 3238, and lower knife tab 3246; which may be substantially similar to vertical column 235, upper knife tab 238, and lower knife tab 246 described above, respectively, with differences elaborated below. Therefore, upper knife tab 3238 and lower knife tab 3246 may actuate within upper knife channel 3224 and lower knife channel 3230, respectively, as knife sled 3236 acutes relative to jaws 3202, 3204 in accordance with the description herein, thereby assisting in pivoting anvil jaw 3204 from the open position into the clamping position.

Staple cartridge 3210 slidably houses cartridge sled 3210A. Staple cartridge defines an elongated knife channel 3215 that is substantially similar to elongated knife channel 215 described above, with differences elaborated herein. Elongated channel 3208 of cartridge jaw 3202 may selectively receive a first staple cartridge 3210 such that an unfired staple cartridge 3210 may be loaded into cartridge jaw 3202 and fired in accordance with the description herein. Next, the spent staple cartridge 3210 may be removed from cartridge jaw 3202 such that a new, unfired staple cartridge staple cartridge 3210 may be loaded into cartridge jaw 3202.

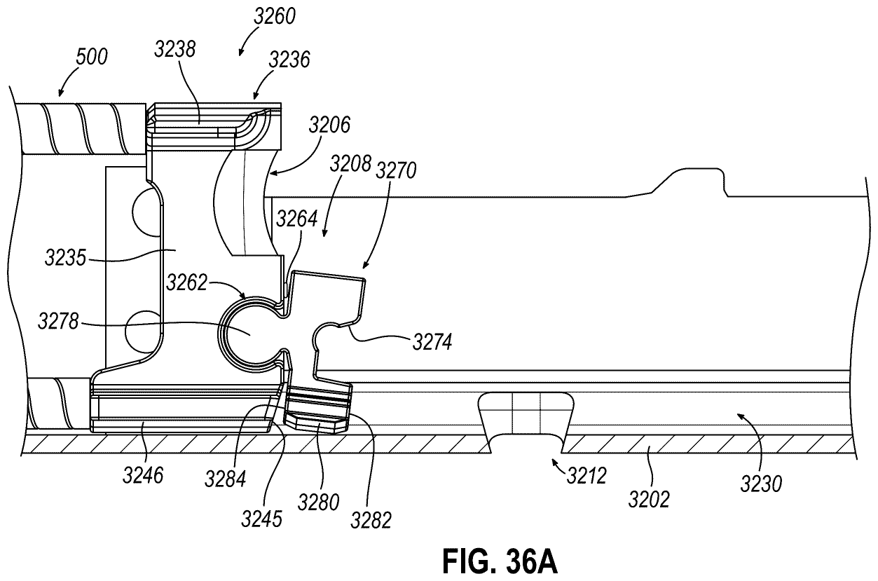

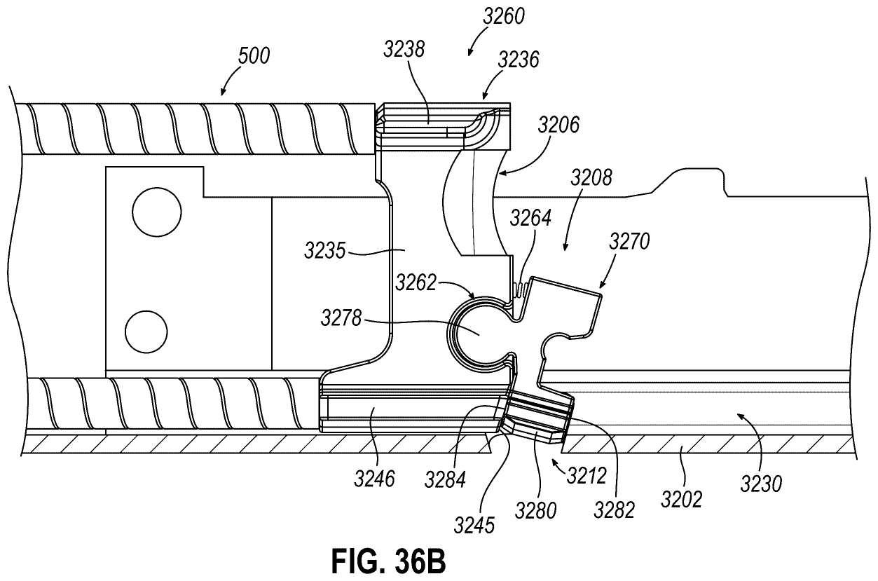

As mentioned above, end effector 3200 includes a lockout assembly 3260 configured to inhibit firing of end effector 3200 in both the spent-cartridge condition and the absent-cartridge condition. In the current illustrative example, lockout assembly 3260 includes a pivoting lockout body 3270 pivotally attached to knife sled 3236, and a biasing element, in the form of a spring 3264, that biases pivoting lockout body 3270 to pivot relative to knife sled 3236 toward a locked-out configuration (see FIG. 36B). Cartridge jaw 3202 also defines a recessed pocket 3212. Recessed pocket 3212 is located distally relative to a pre-fired cartridge sled 3210A when staple cartridge 3210 is suitably loaded within cartridge jaw 3202. While in the current example, recessed pocket 3212 is shown extending between an exterior surface of cartridge jaw 3202 and lower knife channel 3230 of cartridge jaw 3202 (e.g., a through hole), this is merely optional. In some instances, recessed pocket 3212 has a floor such that pocket 3212 does not extend into an exterior surface of cartridge jaw 3202.

As will be described in greater detail below, when a pre-fired staple cartridge 3210 is suitably loaded into cartridge jaw 3202, pivoting lockout body 3270 is configured to engage a pre-fired cartridge sled 3210A as knife 3206 actuates distally from a proximal position, thereby bypassing recessed pocket 3212 defined by cartridge jaw 3202. As will also be described in greater detail below, if a spent-cartridge 3210 is loaded into cartridge jaw 3202 (i.e., a spent-cartridge condition), or a cartridge 3210 is absent from cartridge jaw 3202 (i.e., an absent-cartridge condition), spring 3264 will drive pivoting lockout body 3270 relative to knife sled 3236 into recessed pocket 3212, thereby inhibiting further distal actuation of knife 3206.

Spring 3264 is substantially similar to spring 1264 described above. Therefore, spring 3264 biases pivoting lockout body 3270 toward a lockout-out position (see FIG. 36B).

Knife sled 3236 defines a lateral cutout 3262 on a side of vertical column 3235. Lateral cutout 3262 pivotally houses a substantially circular proximal projection 3278 of pivoting lockout body 3270. Engagement between lateral cutout 3263 and proximal projection 3278 pivotally couples lockout body 3270 to knife sled 3236, thereby allowing lockout body 3270 to pivot relative to knife sled 3236 between a pre-fired configuration (see FIGS. 35A and 36A), an unlocked configuration (see FIGS. 35B-35C), and a locked-out configuration (see FIG. 36B) in accordance with the teachings herein. The pivot axis of pivoting lockout body 3270 is located vertically between push coil 508, 510 of knife firing system. Of course, lockout body 3270 may be pivotally coupled to knife sled 3236 via any other suitable means as would be apparent to one skilled in the art in view of the teachings herein.

Pivoting lockout body 3270 includes an upper section 3272 and a lower section 3280; which may be substantially similair to upper section 1272 and lower section 1280 described above, with differences elaborated herein. In particular, upper section has a substantially circular proximal projection 3278 as compared to the pair of arched proximal projections 1278 described above. The substantially circular geometry of proximal project 3278 limits movement of pivoting lockout body 3270 to pivotal movement, as compared to lockout body 1270 described above which translates and pivots relative to knife sled 1236.

Upper section includes a lockout bypass surface 3274 and a distal sled driving surface 3276; which are substantially similar to lockout bypass surface 1274 and distal sled driving surface 1276 described above. Lower section 3280 includes a distal lockout surface 3292 and a proximal lockout surface 3284, which are substantially similar to distal lockout surface 1282 and proximal lockout surface 1284 described above. Additionally, knife sled includes a lockout engagement surface 3245 that is substantially similar to lockout engagement surface 1245 described above.

FIG. 35A-35C show an illustrative firing of knife 3206 while an unfired staple cartridge 3210 is suitably loaded into end effector 3200; which may be substantially similar to the illustrative firing of end effector 1200 with an unfired staple cartridge 1210 suitably loaded thereon. Therefore, as knife 3206 is actuated distally from the position shown in FIG. 35A to the position shown in FIG. 35B, lockout bypass surface 3274 engages cartridge sled 3210A in accordance with the description herein, thereby inhibiting spring 3264 from driving pivoting lockout body 3270 into the locked-out configuration. Additionally, distal sled driving surface 3276 suitably engages cartridge sled 3210A in accordance with the description herein. Next, as shown in FIG. 35C, with pivoting lockout body 3270 in the unlocked configuration, knife 3206 and cartridge sled 3210A may be fired distally in accordance with the description herein, effectively bypassing the recessed pocket 3212 of cartridge jaw 3202.

FIGS. 36A-36B show an illustrative firing of knife 3206 while a staple cartridge 3210 is not present (i.e., an absent-cartridge condition). It should be understood that while FIGS. 36A-36B show an absent-cartridge condition, the same effect may take place when a spent cartridge condition is present. As mentioned above, in a spent cartridge condition, cartridge sled 3210A would be distal relative to the recessed pocket 3212, effectively rendering the same result as the absent-cartridge condition described below.

As shown in FIG. 36A, with no cartridge 3210 loaded, cartridge sled 3210A is absent and not interposed between pivoting lockout body 3270 and recessed pocket 3212. Pivoting lockout body 3270 is in the pre-fired configuration. Therefore, spring 3264 biases pivoting lockout body 3270 such that a section of lower portion 3280 engages a lower surface of lower knife channel 3230. As shown in FIG. 36B, if knife 3206 is fired in the absent-cartridge condition (or the spent-cartridge condition), spring 3264 biases pivoting lockout body 3270 downward as lockout body 3270 reaches a position adjacent to recessed pocket 3212. Distal lockout surface 3282 is effectively aligned with recessed pocket 3212 such that distal lockout surface 3282 engages recessed pocket 3212, thereby locking out knife 3206 from further distal actuation in accordance with the description herein.

While pivoting lockout body 3270 is in the pre-fired configuration (see FIGS. 35A and 36A) and the unlocked configuration (see FIG. 35B), the entirety of pivoting lockout body 3270 is located vertically between push coils 508, 510. While pivoting lockout body 3270 is in the locked-out configuration (see FIG. 36B), a portion of pivoting lockout body 3270 is located vertically outside the confines of push coils 508, 510.

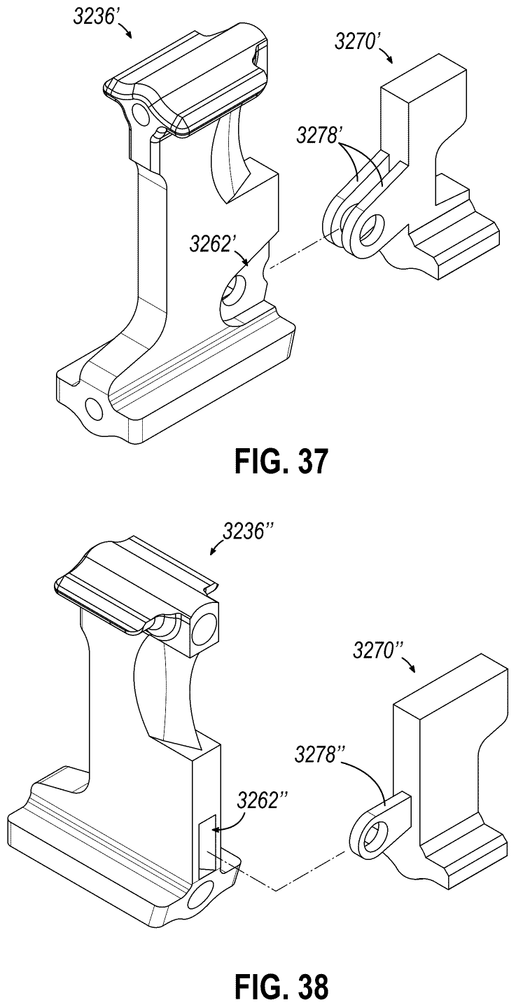

As mentioned herein, lockout bodies may be pivotally coupled to knife sleds using any suitable structures as would be apparent to one skilled in the art in view of the teachings herein. FIGS. 37-38 show illustrative alternative ways to pivotally couple such components together. FIG. 37 shows a modified knife sled 3236′ and modified lockout body 3270′ that may be readily incorporated into any lockout assembly described herein. Modified knife sled 3236′ includes a modified cutout 3262′ with a pivot through hole extending between lateral sides thereof. Further, modified lockout body 3270′ includes a pair or proximally extending projections 3278′ that complement cutout 3262′ and also have pivot through holes. Pivot through holes may receive a pin in order to pivotally couple with each other.

FIG. 38 shows another modified knife sled 3236″ and modified lockout body 3270″ that may be readily incorporated into any lockout assembly described herein. Modified knife sled 3236″ includes a modified cutout 3262″ located on a distally facing surface thereof. Further, modified lockout body 3270″ includes a single proximally extending projection 3278″ that fits within cutout 3262′ and also has a pivot through hole. Pivot through hole may receive a pin in order to pivotally couple sled 3236″ with lockout body 3270″.

It should be understood that pivoting locking bodies described herein may be formed of multiple pieces and may interact with knife sleds described herein utilizing any suitable structures as would be apparent to one skilled in the art in view of the teachings herein.

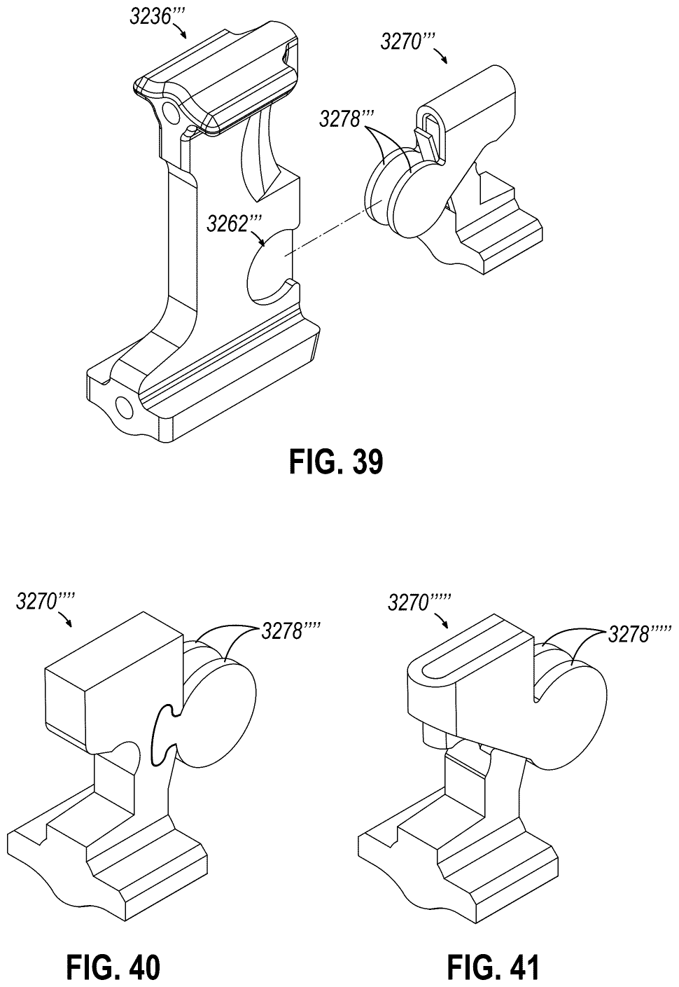

FIG. 39 shows another modified knife sled 3236′″ and modified lockout body 3270′″ that may be readily incorporated into any lockout assembly described herein. Modified knife sled 3236′″ includes a pair of recessed cutouts 3262′″ that are configured to receive a first piece of lockout body 3270′″. The first piece of lockout body 3270′″ may vertically attach to a second piece of lockout body 3270′″ having a torsion spring.

FIG. 40 shows another modified lockout body 3270″″ being formed of multiple pieces that coupled with each other via a puzzle piece relationship, with proximally extending projections 3278″″ being separated from the rest of lockout body 3270″″.

FIG. 41 shows another modified lockout body 3270′″ that includes proximally extending projections 3278′″″ that may couple with the rest of lockout body 3270′″″ via longitudinal motion relative to each other.

E. Fifth Illustrative End Effector with Lockout Assembly

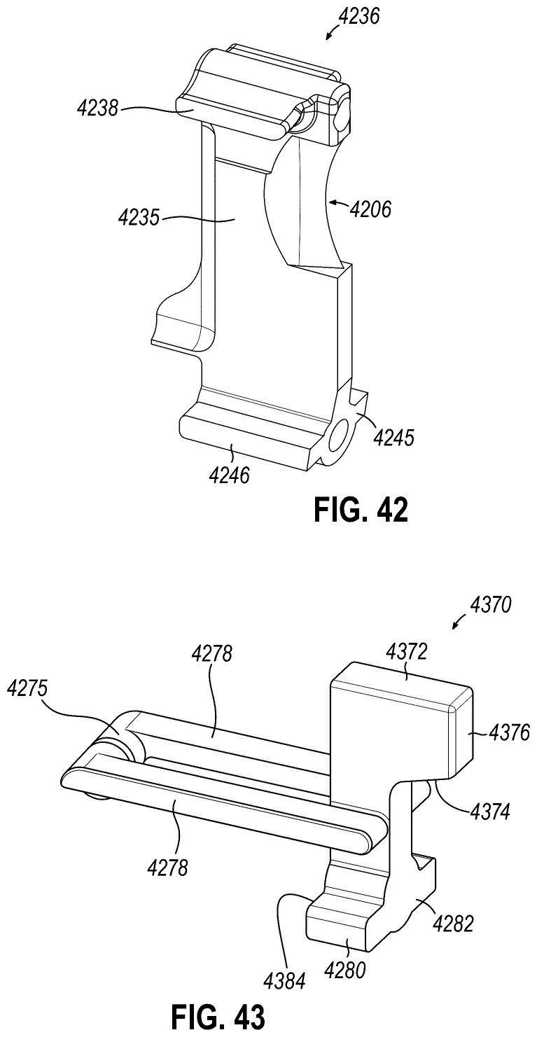

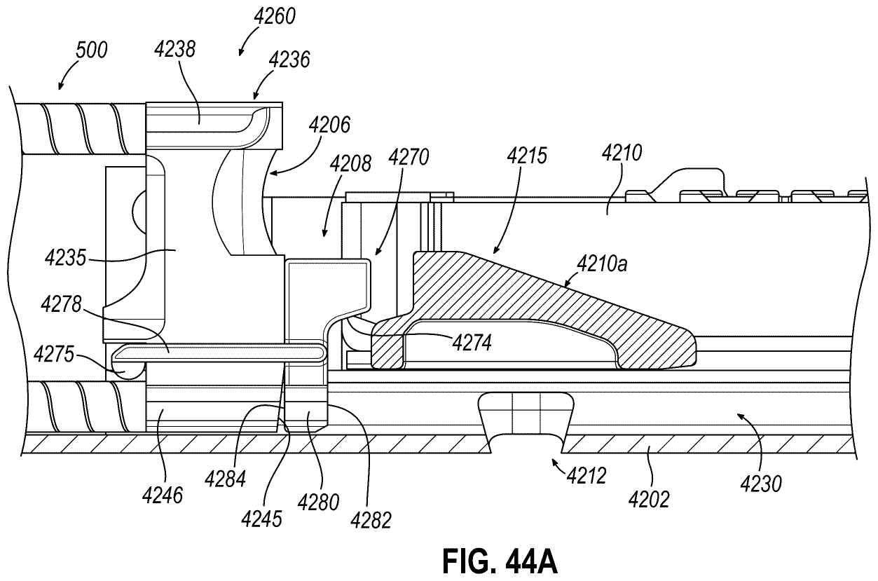

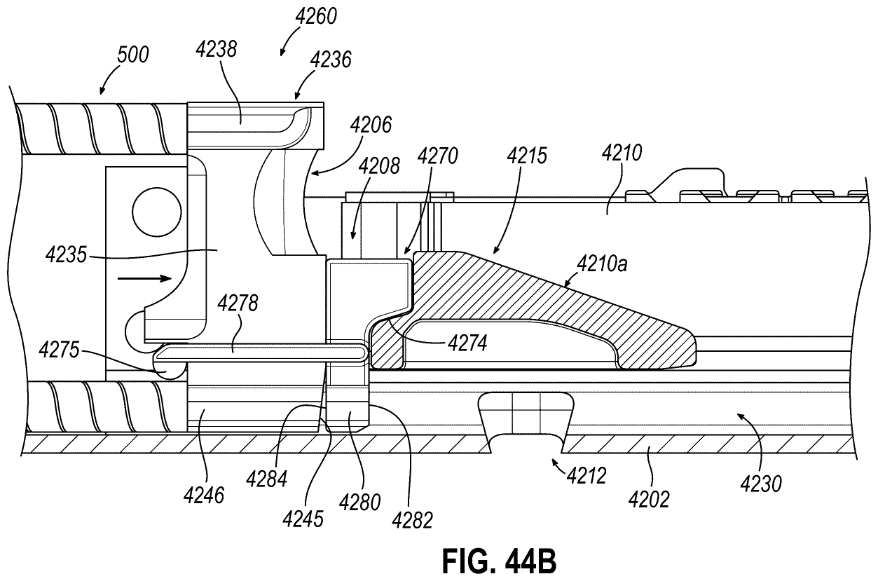

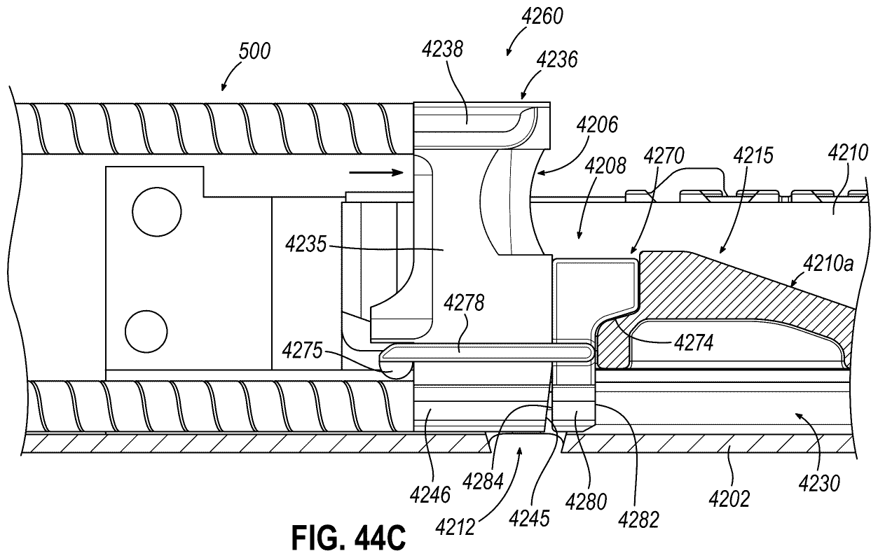

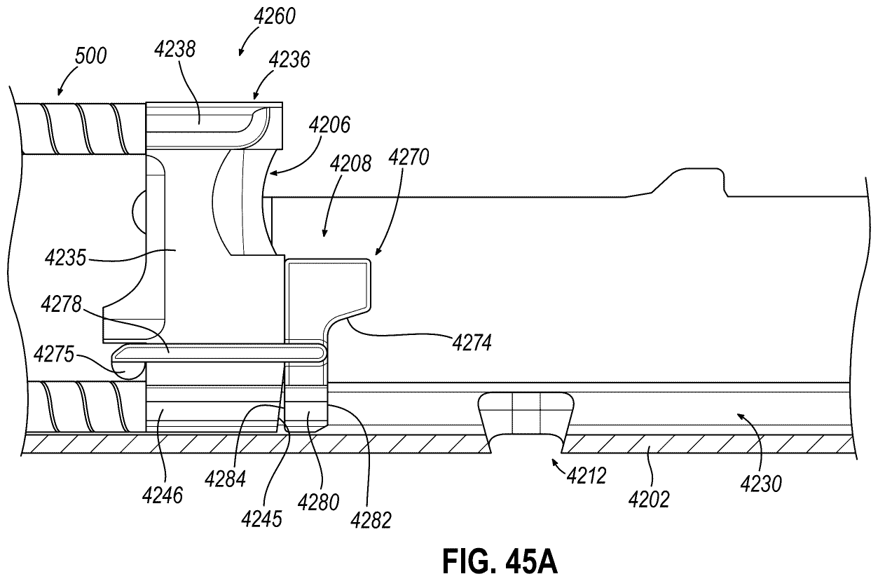

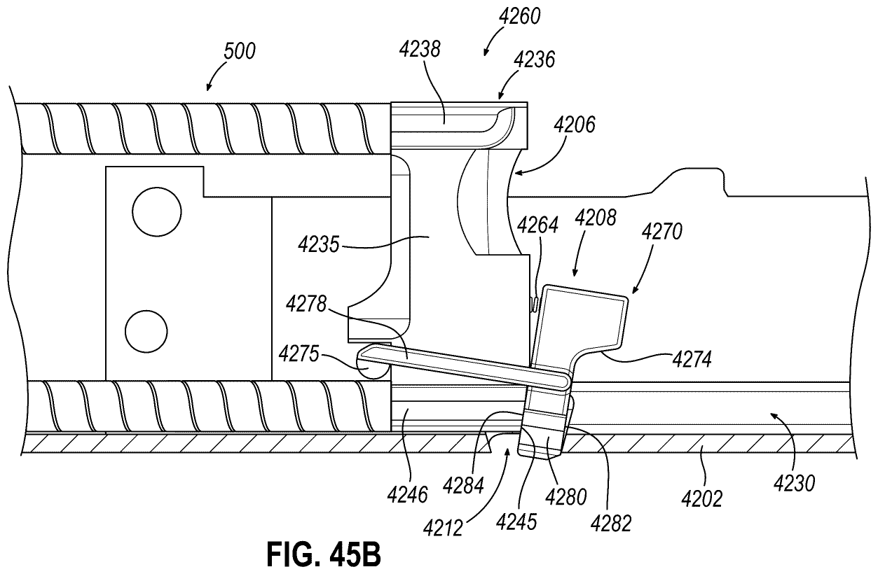

FIGS. 44A-45B show an illustrative end effector 4200 that may be readily incorporated into surgical instrument 1000 in replacement of end effector 200 described above, while FIGS. 42-43 show an illustrative knife 4206 and pivoting lockout body 4270, respectively, of end effector 4200. Therefore, end effector 4200 may be substantially similar to end effector 200, 1200, 2200, 2300, 3200 described above, with differences elaborated herein. In particular, as will be described in greater detail below, end effector 4200 includes a lockout assembly 4260 configured to inhibit firing of end effector 4200 in the spent-cartridge condition and the absent-cartridge condition.

End effector 4200 includes a cartridge jaw 4202, an anvil jaw 4204, a staple cartridge 4210, a cartridge sled 4210A, a knife 4206, and knife sled 4236; which may be substantially similar to cartridge jaw 202, anvil jaw 204, staple cartridge 210, cartridge sled 210A, knife 206, and knife sled 236 described above, respectively, with differences elaborated herein. Therefore, cartridge jaw 4202 and anvil jaw 4204 are pivotally coupled to each other and may pivot relative to each other between an open position (similar to end effector 200 shown in FIG. 8A) and a clamping position (similar to end effector 200 shown in FIGS. 8C-8D), in response to movement of knife sled 4236 in accordance with the description herein.

Knife 4206 and knife sled 4236 are suitably attached to knife firing subsystem 500 such that push coils 508, 510 extend proximally from upper knife tab 4238 and lower knife tab 4246, respectively.

Anvil jaw (not shown) includes a longitudinally extending upper knife channel (not shown) that may be substantially similar to longitudinally extending upper knife channel 224 described above. It should be understood that anvil jaw is omitted for purposes of clarity. Similarly, cartridge jaw 4202 includes an elongated channel 4208 and a lower knife channel 4230, which may be substantially similar to elongate channel 208, and lower knife channel 230 described above, respectively, with differences elaborated below.

Knife sled 4236 includes a vertical column 4235, an upper knife tab 4238, and lower knife tab 4246; which may be substantially similar to vertical column 235, upper knife tab 238, and lower knife tab 246 described above, respectively, with differences elaborated below. Therefore, upper knife tab 4238 and lower knife tab 4246 may actuate within upper knife channel 4224 and lower knife channel 4230, respectively, as knife sled 4236 acutes relative to jaws 4202, 4204 in accordance with the description herein, thereby assisting in pivoting anvil jaw 4204 from the open position into the clamping position.

Staple cartridge 4210 slidably houses cartridge sled 4210A. Staple cartridge defines an elongated knife channel 4215 that is substantially similar to elongated knife channel 215 described above, with differences elaborated herein. Elongated channel 4208 of cartridge jaw 4202 may selectively receive a first staple cartridge 4210 such that an unfired staple cartridge 4210 may be loaded into cartridge jaw 4202 and fired in accordance with the description herein. Next, the spent staple cartridge 4210 may be removed from cartridge jaw 4202 such that a new, unfired staple cartridge staple cartridge 4210 may be loaded into cartridge jaw 4202.

As mentioned above, end effector 4200 includes a lockout assembly 4260 configured to inhibit firing of end effector 4200 in both the spent-cartridge condition and the absent-cartridge condition. In the current illustrative example, lockout assembly 4260 includes a pivoting lockout body 4270 pivotally attached to knife sled 4236, and a biasing element, in the form of a spring 4264, that biases pivoting lockout body 4270 to pivot relative to knife sled 4236 toward a locked-out configuration (see FIG. 45B). Cartridge jaw 4202 also defines a recessed pocket 4212. Recessed pocket 4212 is located distally relative to a pre-fired cartridge sled 4210A when staple cartridge 4210 is suitably loaded within cartridge jaw 4202. While in the current example, recessed pocket 4212 is shown extending between an exterior surface of cartridge jaw 4202 and lower knife channel 4230 of cartridge jaw 4202 (e.g., a through hole), this is merely optional. In some instances, recessed pocket 4212 has a floor such that pocket 4412 does not extend into an exterior surface of cartridge jaw 4202.

As will be described in greater detail below, when a pre-fired staple cartridge 4210 is suitably loaded into cartridge jaw 4202, pivoting lockout body 4270 is configured to engage a pre-fired cartridge sled 4210A as knife 4206 actuates distally from a proximal position, thereby bypassing recessed pocket 4212 defined by cartridge jaw 4202. As will also be described in greater detail below, if a spent-cartridge 4210 is loaded into cartridge jaw 4202 (i.e., a spent-cartridge condition), or a cartridge 4210 is absent from cartridge jaw 4202 (i.e., an absent-cartridge condition), spring 4264 will drive pivoting lockout body 4270 relative to knife sled 4236 into recessed pocket 4212, thereby inhibiting further distal actuation of knife 4206.

Spring 4264 is substantially similar to spring 1264 described above. Therefore, spring 4264 biases pivoting lockout body 4270 toward a lockout-out position (see FIG. 45B).

An upper section 4272 of pivoting lockout body 4270 includes a pair of legs 4278 each terminating into a pivot location 4275. Legs 4278 are resiliently flexible. The sections of each leg 4278 terminating into the pivot location 4275 may flex away from each other during assembly such that legs 4278 may be inserted around vertical column 4235 of knife sled 4236. Once pivot locations 4275 reach a location that is proximal relative to vertical column 4235, the resilient nature of legs 4278 urges pivot locations 4275 toward each other, thereby pivotally coupling lockout body 4270 to knife sled 4236 at pivot location 4275. Therefore, lockout body 4270 pivots relative to knife sled 4236 about pivot location 4275 between a pre-fired configuration (see FIGS. 44A and 45A), an unlocked configuration (see FIGS. 44B-44C), and a locked-out configuration (see FIG. 45B) in accordance with the teachings herein. The pivot axis of pivoting lockout body 4270 is located vertically between push coil 508, 510 of knife firing system. Of course, lockout body 4270 may be pivotally coupled to knife sled 4236 via any other suitable means as would be apparent to one skilled in the art in view of the teachings herein.

Pivoting lockout body 4270 includes an upper section 4272 and a lower section 4280; which may be substantially similair to upper section 1272 and lower section 1280 described above, with differences elaborated herein. In particular, upper section has a substantially resilient legs 4278 that pivotally couple lockout body 4270 to knife sled 4236 as compared the pair of arched proximal projections 1278 described above.

Upper section includes a lockout bypass surface 4274 and a distal sled driving surface 4276; which are substantially similar to lockout bypass surface 1274 and distal sled driving surface 1276 described above. Lower section 4280 includes a distal lockout surface 4292 and a proximal lockout surface 4284, which are substantially similar to distal lockout surface 1282 and proximal lockout surface 1284 described above. Additionally, knife sled includes a lockout engagement surface 4245 that is substantially similar to lockout engagement surface 1245 described above.

FIG. 44A-44C show an illustrative firing of knife 4206 while an unfired staple cartridge 4210 is suitably loaded into end effector 4200; which may be substantially similar to the illustrative firing of end effector 1200 with an unfired staple cartridge 1210 suitably loaded thereon. Therefore, as knife 4206 is actuated distally from the position shown in FIG. 44A to the position shown in FIG. 44B, lockout bypass surface 4274 engages cartridge sled 4210A in accordance with the description herein, thereby inhibiting spring 4264 from driving pivoting lockout body 4270 into the locked-out configuration. Additionally, distal sled driving surface 4276 suitably engages cartridge sled 4210A in accordance with the description herein. Next, as shown in FIG. 44C, with pivoting lockout body 4270 in the unlocked configuration, knife 4206 and cartridge sled 4210A may be fired distally in accordance with the description herein, effectively bypassing the recessed pocket 4212 of cartridge jaw 4202.

FIGS. 45A-45B show an illustrative firing of knife 4206 while a staple cartridge 4210 is not present (i.e., an absent-cartridge condition). It should be understood that while FIGS. 45A-45B show an absent-cartridge condition, the same effect may take place when a spent cartridge condition is present. As mentioned above, in a spent cartridge condition, cartridge sled 4210A would be distal relative to the recessed pocket 4212, effectively rendering the same result as the absent-cartridge condition described below.

As shown in FIG. 45A, with no cartridge 4210 loaded, cartridge sled 4210A is absent and not interposed between pivoting lockout body 4270 and recessed pocket 4212. Pivoting lockout body 4270 is in the pre-fired configuration. Therefore, spring 4264 biases pivoting lockout body 4270 such that a section of lower portion 4280 engages a lower surface of lower knife channel 4230. As shown in FIG. 45B, if knife 4206 is fired in the absent-cartridge condition (or the spent-cartridge condition), spring 4264 biases pivoting lockout body 4270 downward as lockout body 4270 reaches a position adjacent to recessed pocket 4212. Distal lockout surface 4282 is effectively aligned with recessed pocket 4212 such that distal lockout surface 4282 engages recessed pocket 4212, thereby locking out knife 4206 from further distal actuation in accordance with the description herein.

While pivoting lockout body 4270 is in the pre-fired configuration (see FIGS. 44A and 45A) and the unlocked configuration (see FIG. 44B), the entirety of pivoting lockout body 4270 is located vertically between push coils 508, 510. While pivoting lockout body 4270 is in the locked-out configuration (see FIG. 45B), a portion of pivoting lockout body 4270 is located vertically outside the confines of push coils 508, 510.

F. Sixth Illustrative End Effector with Lockout Assembly

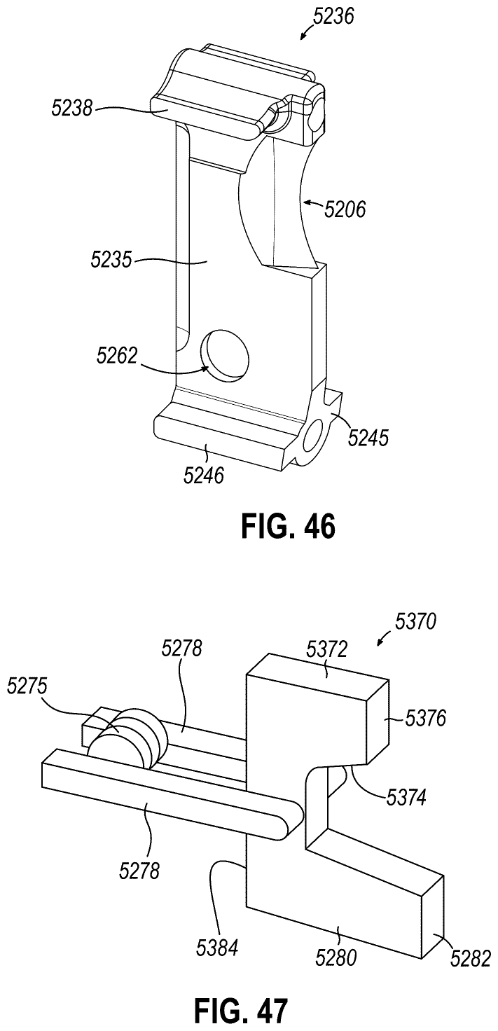

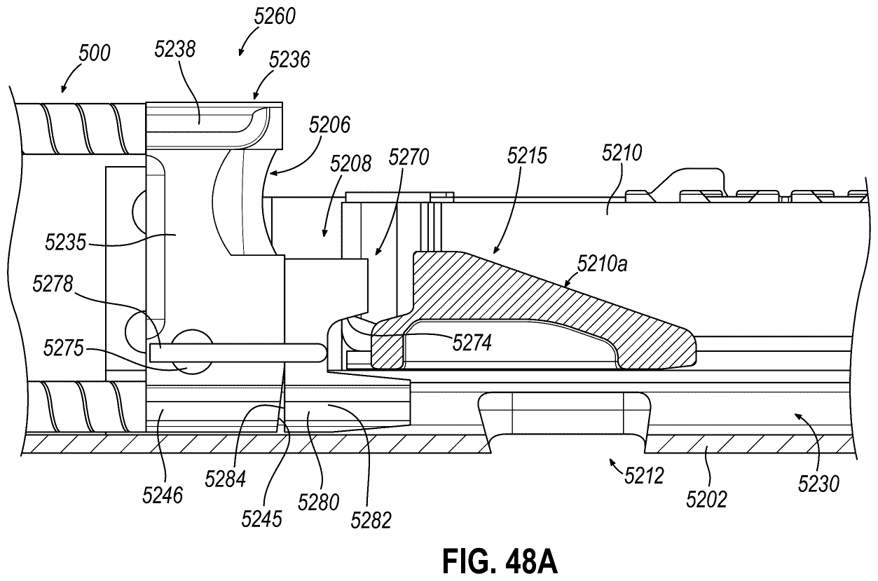

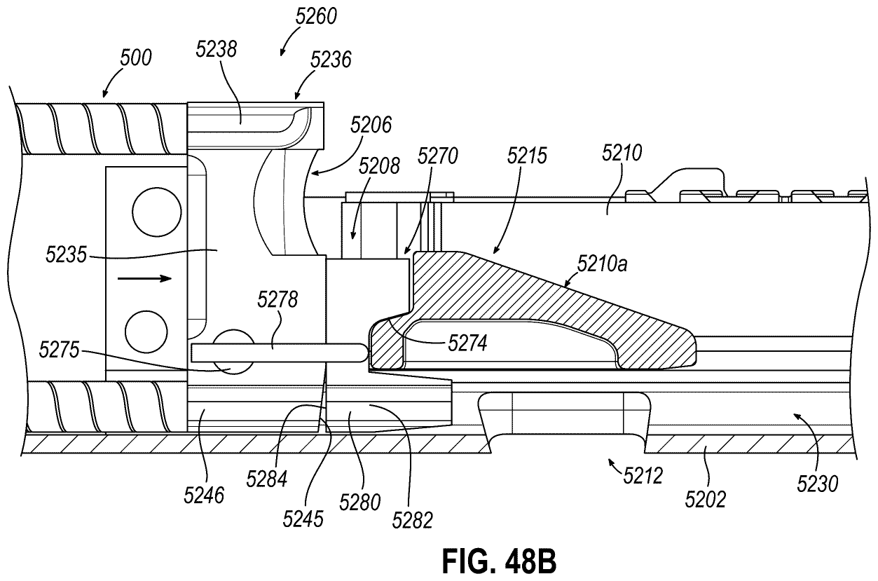

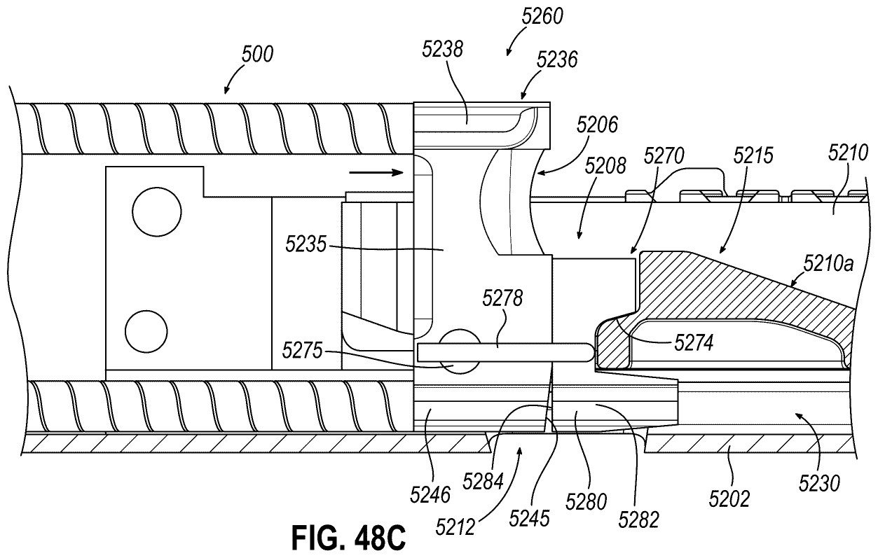

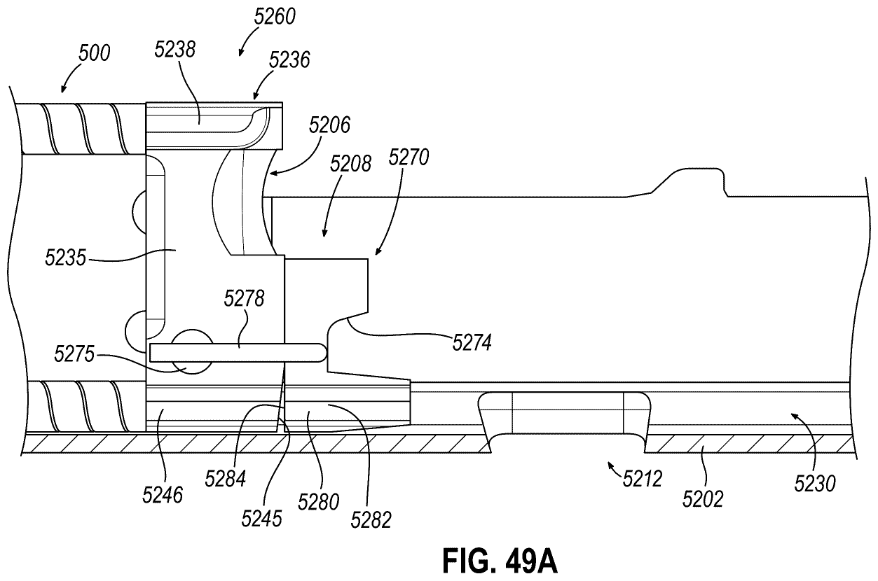

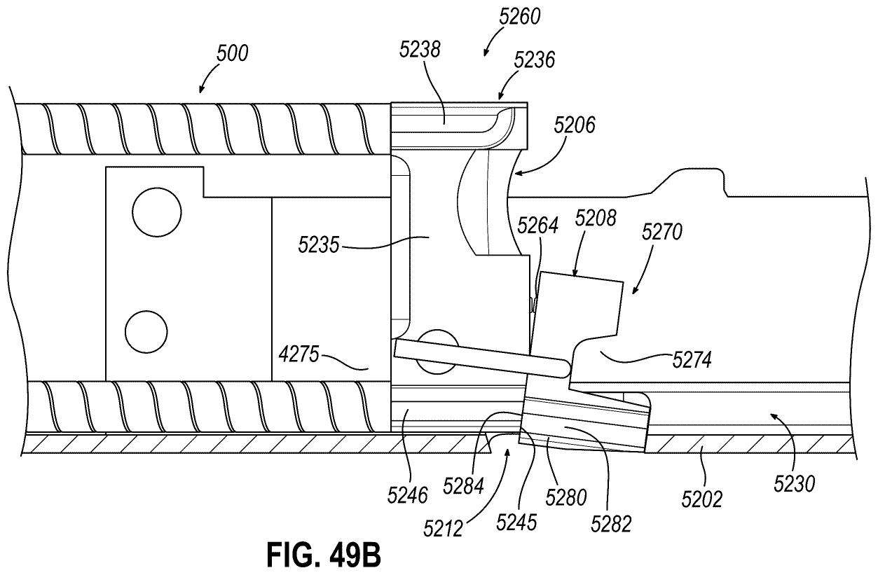

FIGS. 48A-49B show an illustrative end effector 5200 that may be readily incorporated into surgical instrument 1000 in replacement of end effector 200 described above, while FIGS. 46-47 show an illustrative knife 5206 and pivoting lockout body 5270, respectively, of end effector 5200. Therefore, end effector 5200 may be substantially similar to end effector 200, 1200, 2200, 2300, 3200, 4200 described above, with differences elaborated herein. In particular, as will be described in greater detail below, end effector 5200 includes a lockout assembly 5260 configured to inhibit firing of end effector 5200 in the spent-cartridge condition and the absent-cartridge condition.

End effector 5200 includes a cartridge jaw 5202, an anvil jaw 5204, a staple cartridge 5210, a cartridge sled 5210A, a knife 5206, and knife sled 5236; which may be substantially similar to cartridge jaw 202, anvil jaw 204, staple cartridge 210, cartridge sled 210A, knife 206, and knife sled 236 described above, respectively, with differences elaborated herein. Therefore, cartridge jaw 5202 and anvil jaw 5204 are pivotally coupled to each other and may pivot relative to each other between an open position (similar to end effector 200 shown in FIG. 8A) and a clamping position (similar to end effector 200 shown in FIGS. 8C-8D), in response to movement of knife sled 5236 in accordance with the description herein.

Knife 5206 and knife sled 5236 are suitably attached to knife firing subsystem 500 such that push coils 508, 510 extend proximally from upper knife tab 5238 and lower knife tab 5246, respectively.

Anvil jaw (not shown) includes a longitudinally extending upper knife channel (not shown) that may be substantially similar to longitudinally extending upper knife channel 224 described above. It should be understood that anvil jaw is omitted for purposes of clarity. Similarly, cartridge jaw 5202 includes an elongated channel 5208 and a lower knife channel 5230, which may be substantially similar to elongate channel 208, and lower knife channel 230 described above, respectively, with differences elaborated below.

Knife sled 5236 includes a vertical column 5235, an upper knife tab 5238, and lower knife tab 5246; which may be substantially similar to vertical column 235, upper knife tab 238, and lower knife tab 246 described above, respectively, with differences elaborated below. Therefore, upper knife tab 5238 and lower knife tab 5246 may actuate within upper knife channel 5224 and lower knife channel 5230, respectively, as knife sled 5236 acutes relative to jaws 5202, 5204 in accordance with the description herein, thereby assisting in pivoting anvil jaw 5204 from the open position into the clamping position.

Staple cartridge 5210 slidably houses cartridge sled 5210A. Staple cartridge defines an elongated knife channel 5215 that is substantially similar to elongated knife channel 215 described above, with differences elaborated herein. Elongated channel 5208 of cartridge jaw 5202 may selectively receive a first staple cartridge 5210 such that an unfired staple cartridge 5210 may be loaded into cartridge jaw 5202 and fired in accordance with the description herein. Next, the spent staple cartridge 5210 may be removed from cartridge jaw 5202 such that a new, unfired staple cartridge staple cartridge 5210 may be loaded into cartridge jaw 5202.

As mentioned above, end effector 5200 includes a lockout assembly 5260 configured to inhibit firing of end effector 5200 in both the spent-cartridge condition and the absent-cartridge condition. In the current illustrative example, lockout assembly 5260 includes a pivoting lockout body 5270 pivotally attached to knife sled 5236, and a biasing element, in the form of a spring 5264, that biases pivoting lockout body 5270 to pivot relative to knife sled 5236 toward a locked-out configuration (see FIG. 49B). Cartridge jaw 5202 also defines a recessed pocket 5212. Recessed pocket 5212 is located distally relative to a pre-fired cartridge sled 5210A when staple cartridge 5210 is suitably loaded within cartridge jaw 5202. While in the current example, recessed pocket 5212 is shown extending between an exterior surface of cartridge jaw 5202 and lower knife channel 5230 of cartridge jaw 5202 (e.g., a through hole), this is merely optional. In some instances, recessed pocket 5212 has a floor such that pocket 5412 does not extend into an exterior surface of cartridge jaw 5202.

As will be described in greater detail below, when a pre-fired staple cartridge 5210 is suitably loaded into cartridge jaw 5202, pivoting lockout body 5270 is configured to engage a pre-fired cartridge sled 5210A as knife 5206 actuates distally from a proximal position, thereby bypassing recessed pocket 5212 defined by cartridge jaw 5202. As will also be described in greater detail below, if a spent-cartridge 5210 is loaded into cartridge jaw 5202 (i.e., a spent-cartridge condition), or a cartridge 5210 is absent from cartridge jaw 5202 (i.e., an absent-cartridge condition), spring 5264 will drive pivoting lockout body 5270 relative to knife sled 5236 into recessed pocket 5212, thereby inhibiting further distal actuation of knife 5206.

Spring 5264 is substantially similar to spring 4264 described above. Therefore, spring 5264 biases pivoting lockout body 5270 toward a lockout-out position (see FIG. 49B).

An upper section 5272 of pivoting lockout body 5270 includes a pair of legs 5278 each terminating into a pivot location 5275. Legs 5278 are resiliently flexible. The sections of each leg 5278 terminating into the pivot location 5275 may flex away from each other during assembly such that legs 5278 may be inserted around vertical column 5235 of knife sled 5236 until pivot locations 5275 reach a corresponding pivot recess 5262 defined by the lateral sides of vertical column 5235 of knife sled 5236. Once pivot locations 5275 reach a corresponding pivot recess 5262, the resilient nature of legs 5278 urges pivot locations 5275 into pivot recesses 5262, thereby pivotally coupling lockout body 5270 to knife sled 5236 at pivot location 5275. Therefore, lockout body 5270 pivots relative to knife sled 5236 about pivot location 5275 and pivot recesses 5262 between a pre-fired configuration (see FIGS. 48A and 49A), an unlocked configuration (see FIGS. 48B-48C), and a locked-out configuration (see FIG. 49B) in accordance with the teachings herein. The pivot axis of pivoting lockout body 5270 is located vertically between push coil 508, 510 of knife firing system Of course, lockout body 5270 may be pivotally coupled to knife sled 5236 via any other suitable means as would be apparent to one skilled in the art in view of the teachings herein.

Pivoting lockout body 5270 includes an upper section 5272 and a lower section 5280; which may be substantially similar to upper section 1272 and lower section 1280 described above, with differences elaborated herein. In particular, upper section has a substantially resilient legs 5278 that pivotally couple lockout body 5270 to knife sled 5236 as compared the pair of arched proximal projections 1278 described above.

Upper section includes a lockout bypass surface 5274 and a distal sled driving surface 5276; which are substantially similar to lockout bypass surface 1274 and distal sled driving surface 1276 described above. Lower section 5280 includes a distal lockout surface 5292 and a proximal lockout surface 5284, which are substantially similar to distal lockout surface 1282 and proximal lockout surface 1284 described above. Additionally, knife sled includes a lockout engagement surface 5245 that is substantially similar to lockout engagement surface 1245 described above.

FIG. 48A-48C show an illustrative firing of knife 5206 while an unfired staple cartridge 5210 is suitably loaded into end effector 5200; which may be substantially similar to the illustrative firing of end effector 1200 with an unfired staple cartridge 1210 suitably loaded thereon. Therefore, as knife 5206 is actuated distally from the position shown in FIG. 48A to the position shown in FIG. 48B, lockout bypass surface 5274 engages cartridge sled 5210A in accordance with the description herein, thereby inhibiting spring 5264 from driving pivoting lockout body 5270 into the locked-out configuration. Additionally, distal sled driving surface 5276 suitably engages cartridge sled 5210A in accordance with the description herein. Next, as shown in FIG. 48C, with pivoting lockout body 5270 in the unlocked configuration, knife 5206 and cartridge sled 5210A may be fired distally in accordance with the description herein, effectively bypassing the recessed pocket 5212 of cartridge jaw 5202.

FIGS. 49A-49B show an illustrative firing of knife 5206 while a staple cartridge 5210 is not present (i.e., an absent-cartridge condition). It should be understood that while FIGS. 49A-49B show an absent-cartridge condition, the same effect may take place when a spent cartridge condition is present. As mentioned above, in a spent cartridge condition, cartridge sled 5210A would be distal relative to the recessed pocket 5212, effectively rendering the same result as the absent-cartridge condition described below.

As shown in FIG. 49A, with no cartridge 5210 loaded, cartridge sled 5210A is absent and not interposed between pivoting lockout body 5270 and recessed pocket 5212. Pivoting lockout body 5270 is in the pre-fired configuration. Therefore, spring 5264 biases pivoting lockout body 5270 such that a section of lower portion 5280 engages a lower surface of lower knife channel 5230. As shown in FIG. 49B, if knife 5206 is fired in the absent-cartridge condition (or the spent-cartridge condition), spring 5264 biases pivoting lockout body 5270 downward as lockout body 5270 reaches a position adjacent to recessed pocket 5212. Distal lockout surface 5282 is effectively aligned with recessed pocket 5212 such that distal lockout surface 5282 engages recessed pocket 5212, thereby locking out knife 5206 from further distal actuation in accordance with the description herein.

While pivoting lockout body 5270 is in the pre-fired configuration (see FIGS. 48A and 49A) and the unlocked configuration (see FIG. 48B), the entirety of pivoting lockout body 5270 is located vertically between push coils 508, 510. While pivoting lockout body 5270 is in the locked-out configuration (see FIG. 49B), a portion of pivoting lockout body 5270 is located vertically outside the confines of push coils 508, 510.

G. Seventh Illustrative End Effector with Lockout Assembly

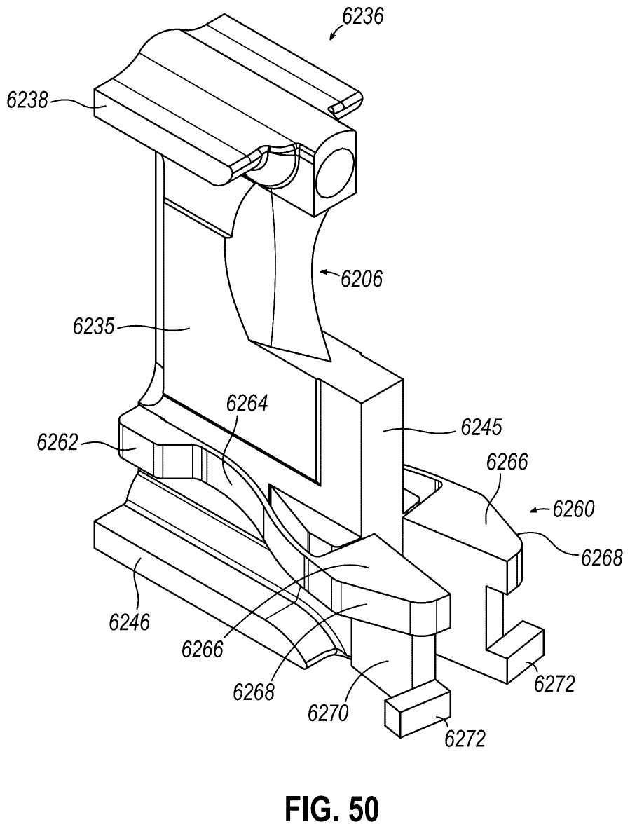

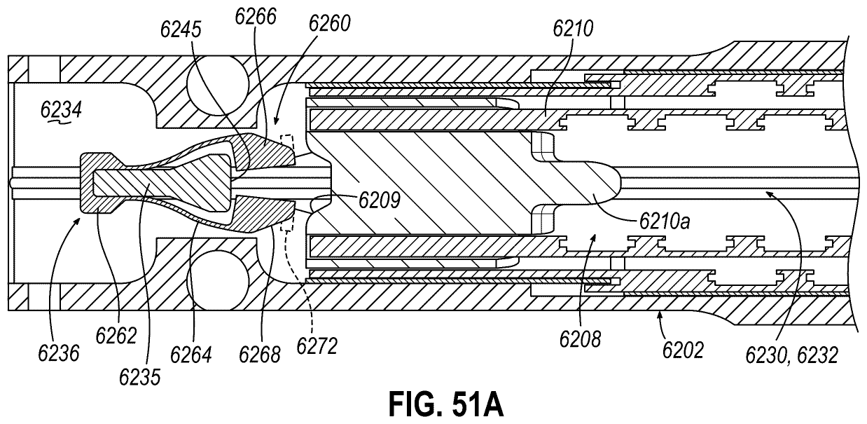

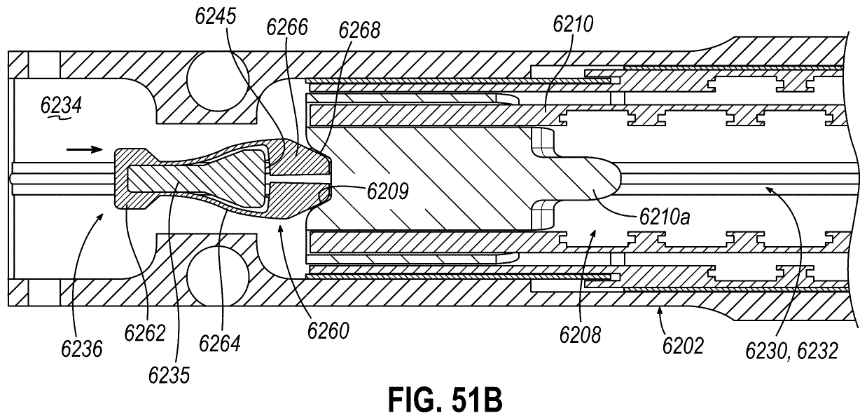

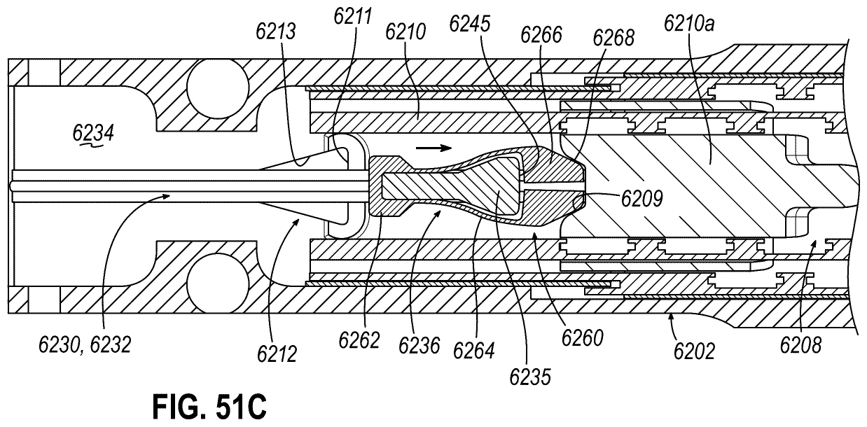

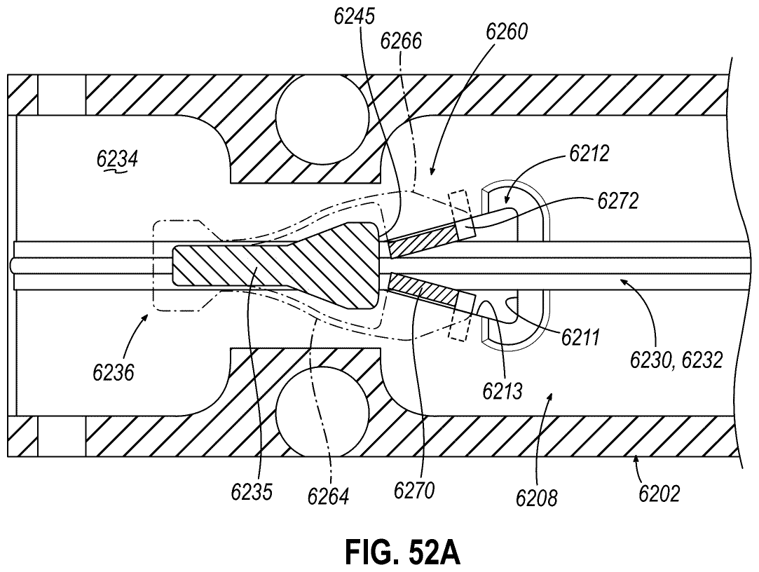

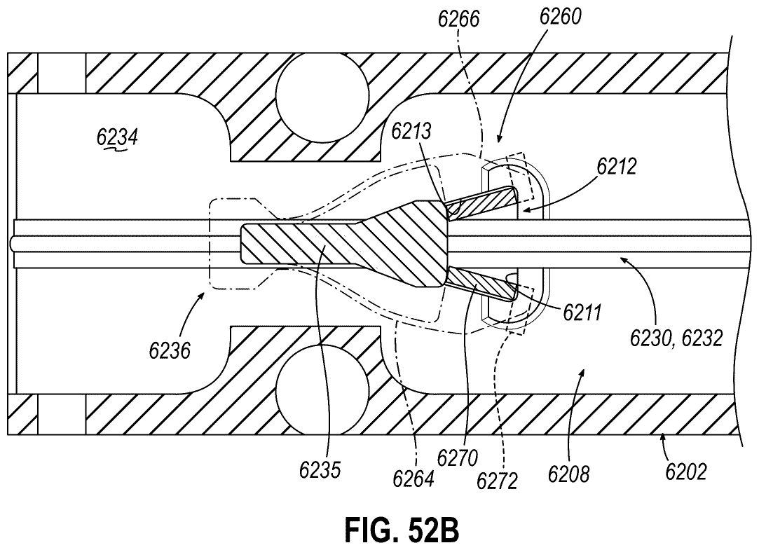

FIGS. 51A-52B show an illustrative end effector 6200 that may be readily incorporated into surgical instrument 1000 in replacement of end effector 200 described above, while FIG. 50 shows an illustrative knife 6206 and lockout ledge engagement body 6270 of end effector 6200. Therefore, end effector 6200 may be substantially similar to end effector 200, 1200, 2200, 2300, 3200, 4200, 5200 described above, with differences elaborated herein. As will be described in greater detail below, end effector 6200 includes a lockout assembly 6260 configured to inhibit firing of end effector 6200 in the spent-cartridge condition and the absent-cartridge condition.

End effector 6200 includes a cartridge jaw 6202, an anvil jaw 6204, a staple cartridge 6210, a cartridge sled 6210A, a knife 6206, and knife sled 6236; which may be substantially similar to cartridge jaw 202, anvil jaw 204, staple cartridge 210, cartridge sled 210A, knife 206, and knife sled 236 described above, respectively, with differences elaborated herein. Therefore, cartridge jaw 6202 and anvil jaw 6204 are pivotally coupled to each other and may pivot relative to each other between an open position (similar to end effector 200 shown in FIG. 8A) and a clamping position (similar to end effector 200 shown in FIGS. 8C-8D), in response to movement of knife sled 6236 in accordance with the description herein.

Anvil jaw (not shown) includes a longitudinally extending upper knife channel (not shown) that may be substantially similar to longitudinally extending upper knife channel 224 described above. It should be understood that anvil jaw is omitted for purposes of clarity. Similarly, cartridge jaw 6202 includes an elongated channel 6208 and a lower knife channel 6230, which may be substantially similar to elongate channel 208, and lower knife channel 230 described above, respectively, with differences elaborated below. Lower knife channel 6230 includes a central lower knife channel portion 6232 and at least one lateral lower knife channel wing 6234; which may be substantially similar to central lower knife channel portion 232 and lower knife channel wing 6234 described above, with differences elaborated below.

Knife sled 6236 includes a vertical column 6235, an upper knife tab 6238, and lower knife tab 6246; which may be substantially similar to vertical column 235, upper knife tab 238, and lower knife tab 246 described above, respectively, with differences elaborated below. Therefore, upper knife tab 6238 and lower knife tab 6246 may actuate within upper knife channel 6224 and lower knife channel 6230, respectively, as knife sled 6236 acutes relative to jaws 6202, 6204 in accordance with the description herein, thereby assisting in pivoting anvil jaw 6204 from the open position into the clamping position.

Staple cartridge 6210 slidably houses cartridge sled 6210A. Staple cartridge defines an elongated knife channel 6215 that is substantially similar to elongated knife channel 215 described above, with differences elaborated herein. Elongated channel 6208 of cartridge jaw 6202 may selectively receive a first staple cartridge 6210 such that an unfired staple cartridge 6210 may be loaded into cartridge jaw 6202 and fired in accordance with the description herein. Next, the spent staple cartridge 6210 may be removed from cartridge jaw 6202 such that a new, unfired staple cartridge staple cartridge 6210 may be loaded into cartridge jaw 6202.

As mentioned above, end effector 6200 includes a lockout assembly 6260 configured to inhibit firing of end effector 6200 in both the spent-cartridge condition and the absent-cartridge condition. In the current illustrative example, lockout assembly 6260 includes a pair of leaf springs 6264 attached to a proximal end of knife sled 6236 at a coupling 6262, and a pair of lockout blocks 6266, each attached to a distal end of a respective leaf spring 6264. Therefore, leaf springs 6264 and lockout blocks 6266 are configured to actuate with knife 6206 in accordance with the description herein.

Each lockout block includes a bypass camming surface 6268 configured to engage a pre-fired cartridge sled 6210A during the firing of knife 6206 in accordance with the description herein. Engaged between bypass camming surface 6268 and a pre-fired cartridge sled 6210A urges lockout blocks 6266 laterally toward each other such that lockout blocks 6266 effectively bypass a lockout ledge 6211 of cartridge jaw 6202, thereby allowing knife 6206 to be fired distally in accordance with the description herein. In other words, engagement between bypass camming surface 6288 and a pre-fired cartridge sled 6120A prevents lockout blocks 6266 from contacting lockout ledge 6211 of cartridge jaw 6202, which allows for knife 6206 to be fired distally in accordance with the description herein.

Additionally, each lockout ledge engagement body 6270 configured to engage a lockout ledge 6211 of cartridge jaw 6202 when knife 6206 is fired in the spent-cartridge condition of the absent-cartridge condition, and transmit the lockout load received from engagement with lockout ledge 6211 onto a lockout ledge engagement surface 6245 of vertical column 6235 of knife sled 6236. Further, each lockout block 6266 includes a lower knife channel body 6272 configured to actuate within lower knife channel 6230.

Leaf springs 6264 bias a respective lockout block 6266 laterally away from each other toward a respective sidewall of cartridge jaw 6202. As shown in both FIGS. 51A and 52A, leaf springs 6264 bias lockout ledge engagement body 6270 to abut against a surface of centrally disposed lower knife channel portion 6232 of lower knife channel 6230 that is in communication with elongated channel 6208 of cartridge jaw 6202. Leaf springs 6264 biases a respective lockout block 6266 toward a lockout-out configuration (see FIG. 52B).

Cartridge jaw 6202 also defines a recessed pocket 6212 having a lockout ledge 6211 and an oblique surface 6213. Recessed pocket 6212 is located distally relative to a pre-fired cartridge sled 5210A when staple cartridge 5210 is suitably loaded within cartridge jaw 5202. Therefore, if a pre-fired staple cartridge 6210 is loaded, lockout blocks 6266 will suitably engage a pre-fired cartridge sled 6210A prior to reaching recessed pocket 6212. Each recessed pocket 6212 is defined by the same portion of cartridge jaw 6202 which a respective lockout ledge engagement body 6270 is biased against by leaf spring 6264. Therefore, if knife 6206 is fired in either the spent-cartridge condition or the absent-cartridge condition, leaf spring 6264 biases lockout ledge engagement body 6270 into recessed pocket 6212 once lockout ledge engagement body 6270 is adjacent to recessed pockets 6212. In particular, oblique surface 6213 initially receives lockout ledge engagement body 6270, thereby allowing engagement body 6270 to flare laterally outward via the laterally outward bias of leaf spring 6264. Then, with lockout ledge engagement body 6270 flared laterally outward, engage body 6270 then engages lockout ledge 6211, which inhibits further distally advancement of engagement body 6270. A proximal end of engagement body 6270 then transfers the load received by lockout ledge 6211 onto knife sled 6236 via engagement with lockout engagement surface 6245 of vertical column 6235.

FIG. 51A-51C show an illustrative firing of knife 6206 while an unfired staple cartridge 6210 is suitably loaded into end effector 6200. As knife 6206 is actuated distally from the position shown in FIG. 51A to the position shown in FIG. 51B, bypass camming surface 6268 of each lockout block 6266 engages a respective lateral inward presented camming surface 6209 cartridge sled 6210A in accordance with the description herein, thereby inhibiting leaf spring 6264 from driving lockout blocks 6266 into the locked-out configuration. Next, as shown in FIG. 51C, with lockout blocks 6266 in the unlocked configuration, knife 6206 and cartridge sled 6210A may be fired distally in accordance with the description herein, effectively bypassing the recessed pockets 6212 of cartridge jaw 6202.

FIGS. 52A-52B show an illustrative firing of knife 6206 while a staple cartridge 6210 is not present (i.e., an absent-cartridge condition). It should be understood that while FIGS. 52A-52B show an absent-cartridge condition, the same effect may take place when a spent cartridge condition is present. As mentioned above, in a spent cartridge condition, cartridge sled 6210A would be distal relative to the recessed pocket 6212, effectively rendering the same result as the absent-cartridge condition described below.

As shown in FIG. 52A, with no cartridge 6210 loaded, cartridge sled 6210A is absent and not interposed between lockout blocks 6266 and recessed pocket 6212. Lockout blocks 6266 are in the pre-fired configuration. Therefore, leaf springs 6264 bias a respective lockout block 6266 against a surface of lower knife channel 6230 in communication with elongated channel 6208. As shown in FIG. 52B, if knife 6206 is fired in the absent-cartridge condition (or the spent-cartridge condition), spring 6264 biases lockout blocks 6266 laterally outward as lockout blocks 6266 reach a position adjacent to recessed pocket 6212. Lockout ledge engagement body 6270 is engaged with recessed pocket 6212 and transmitting that load onto lockout engagement surface 62456 of knife sled 6236, thereby locking out knife 6206 from further distal actuation in accordance with the description herein.

H. Eighth Illustrative End Effector with Lockout Assembly

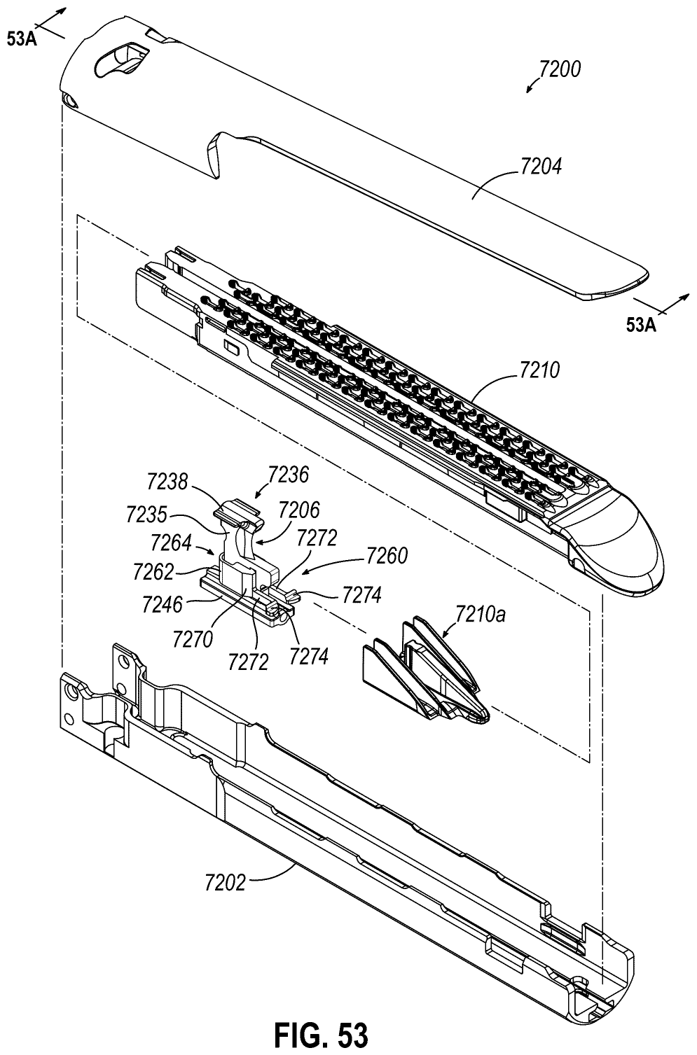

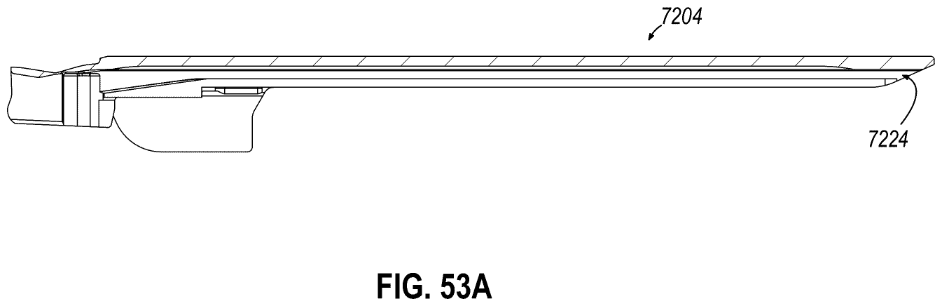

FIGS. 53-55B show an illustrative end effector 7200 that may be readily incorporated into surgical instrument 1000 in replacement of end effector 200 described above. Therefore, end effector 7200 may be substantially similar to end effector 200, 1200, 2200, 2300, 3200, 4200, 5200, 6200 described above, with differences elaborated herein. As will be described in greater detail below, end effector 7200 includes a lockout assembly 7260 configured to inhibit firing of end effector 7200 in the spent-cartridge condition and the absent-cartridge condition.

End effector 7200 includes a cartridge jaw 7202, an anvil jaw 7204, a staple cartridge 7210, a cartridge sled 7210A, a knife 7206, and knife sled 7236; which may be substantially similar to cartridge jaw 202, anvil jaw 204, staple cartridge 210, cartridge sled 210A, knife 206, and knife sled 236 described above, respectively, with differences elaborated herein. Therefore, cartridge jaw 7202 and anvil jaw 7204 are pivotally coupled to each other and may pivot relative to each other between an open position (similar to end effector 200 shown in FIG. 8A) and a clamping position (similar to end effector 200 shown in FIGS. 8C-8D), in response to movement of knife sled 7236 in accordance with the description herein.

As shown in FIG. 53A, anvil jaw 7204 includes a longitudinally extending upper knife channel 7224 that may be substantially similar to longitudinally extending upper knife channel 224 described above. Similarly, cartridge jaw 7202 includes an elongated channel 7208 and a lower knife channel 7230, which may be substantially similar to elongate channel 208, and lower knife channel 230 described above, respectively, with differences elaborated below. A proximal portion of cartridge jaw 7202 defining elongated channel 7208 includes camming lockout surfaces 7212 that oppose each other to define a channel dimensioned to receive knife 7206 and knife sled 7236.

Knife sled 7236 includes a vertical column 7235, an upper knife tab 7238, and lower knife tab 7246; which may be substantially similar to vertical column 235, upper knife tab 238, and lower knife tab 246 described above, respectively, with differences elaborated below. Therefore, upper knife tab 7238 and lower knife tab 7246 may actuate within upper knife channel 7224 and lower knife channel 7230, respectively, as knife sled 7236 acutes relative to jaws 7202, 7204 in accordance with the description herein, thereby assisting in pivoting anvil jaw 7204 from the open position into the clamping position.

Staple cartridge 7210 slidably houses cartridge sled 7210A. Staple cartridge defines an elongated knife channel 7215 that is substantially similar to elongated knife channel 215 described above, with differences elaborated herein. Elongated channel 7208 of cartridge jaw 7202 may selectively receive a first staple cartridge 7210 such that an unfired staple cartridge 7210 may be loaded into cartridge jaw 7202 and fired in accordance with the description herein. Next, the spent staple cartridge 7210 may be removed from cartridge jaw 7202 such that a new, unfired staple cartridge staple cartridge 7210 may be loaded into cartridge jaw 7202.

As mentioned above, end effector 7200 includes a lockout assembly 7260 configured to inhibit firing of end effector 7200 in both the spent-cartridge condition and the absent-cartridge condition. In the current example, lockout assembly 7260 includes a knife clip 7262 that is slidably housed within the distal retainer 324 and a proximal portion of cartridge jaw 7202. As will be described in greater detail below, when an unfired staple cartridge 7210 is suitably loaded into cartridge jaw 7202, knife clip 7262 is configured to release knife sled 7236, thereby allowing distal firing of knife 7206 in accordance with the description herein. However, as will also be described in greater detail below, if a spent-cartridge 7210 is loaded into cartridge jaw 7202 (i.e., a spent-cartridge condition), or a cartridge 7210 is absent from cartridge jaw 7202 (i.e., an absent-cartridge condition), knife clip 7262 will cam against camming lockout surfaces 7212 to thereby compress suitable portions of knife clip 7262 against the side walls of knife sled 7236, thereby inhibiting further distal actuation of knife 7206.

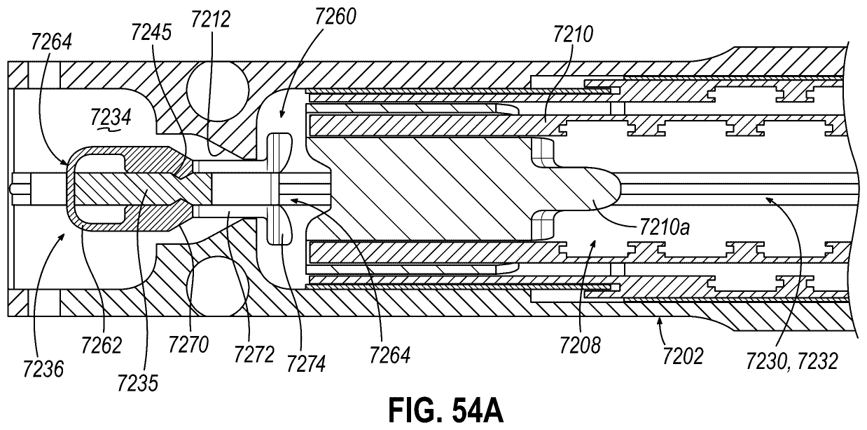

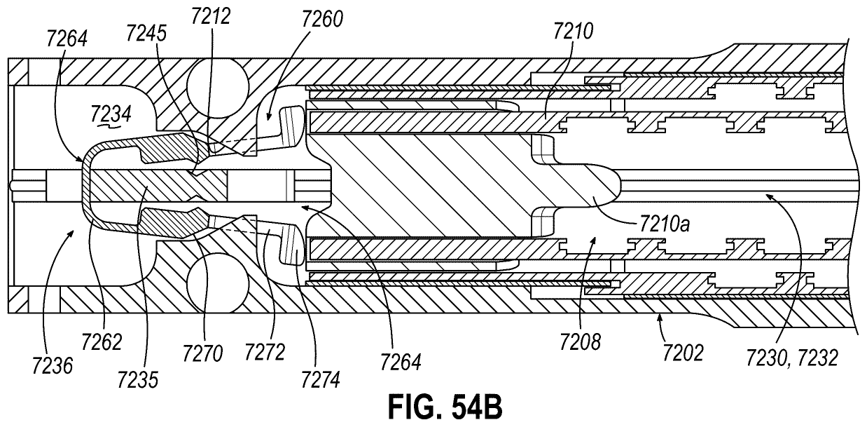

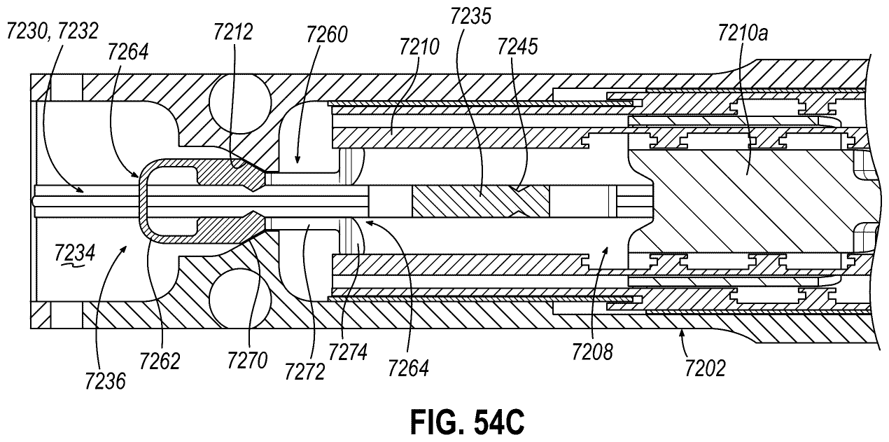

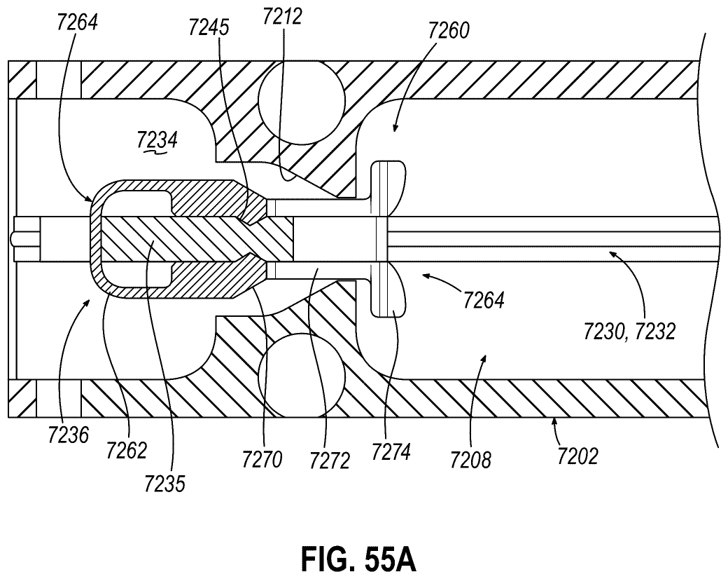

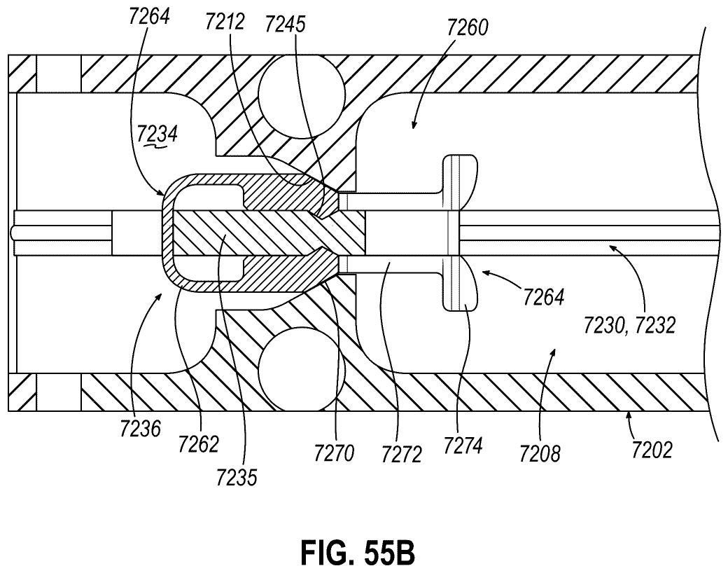

Knife clip 7262 includes a pair of resilient legs 7272 that couple to each other at a closed proximal end 7264 and are spaced apart from each other at an open distal end 7266. Resilient legs 7272 define a knife receiving slot 7268 dimensioned to suitably house knife sled 7236 in the pre-fired position (see FIGS. 54A and 55A). When a pre-fired staple cartridge 7210 is suitably loaded, knife 7206 is configured to acuate distally out of the confines of knife receiving slot 7268 via open distal end 7266, and then actuate proximally back within the confines of knife receiving slot 7268 after being fired in accordance with the description herein. In other words, knife clip 7262 is configured to repeatedly release and receive knife sled 7236 such that knife 7206 may disassociate with knife clip 7262 while fired, and then reassociate with knife clip 7262 once knife 7206 actuated back into the pre-fired position.

An outer surface of each resilient leg 7272 includes a lockout cam surface 7270, while a distal end of each resilient leg 7272 includes a sled engagement surface 7274. Resilient legs 7272 are sufficiently resilient in order to compress laterally inward toward each other (to thereby cinch onto and inhibit distal firing of knife 7206) as well as expand laterally outward away from each other (to thereby release and promote distal firing of knife 7206). While knife sled 7236 is in a proximal position (see FIGS. 54A and 55A), resilient legs 7272 of knife clip 7262 suitably engage a lockout engagement surface 7245 knife sled 7236 such that knife clip 7262 initially actuates distally with knife sled 7236.

Resilient legs 7272 are suitably long enough such that when a pre-fired staple cartridge 7210 is loaded in cartridge jaw 7202 in accordance with the teachings herein, and knife 7206 is initially actuated distally from the proximal position (see FIG. 54A), sled engagement surface 7274 suitably engages a pre-fired cartridge sled 7210A prior to lockout cam surfaces 7270 engaging camming lockout surfaces 7212 of cartridge jaw 7202 (see FIG. 54B). Sled engagement surfaces 7274 have an arched profile such that initial engagement between sled engagement surface 7274 and a pre-fired cartridge sled 7210A laterally expands both resilient legs 7272 away from each other. When resilient legs 7272 laterally expand away from each other, knife clip 7262 releases knife sled 7236, thereby allowing knife 7206 to be fired distally in accordance with the teachings herein (see FIG. 54C).

If a pre-fired staple cartridge 7210 is not loaded into cartridge jaw 7202 (either the spent cartridge condition or the absent-cartridge condition), when knife 7206 is initially actuated distally from the proximal position (see FIG. 55A), sled engagement surfaces 7274 fail to engage a pre-fired cartridge sled 7210A, since no sled 7210A is located at the pre-fired position. Therefore, lockout cam surfaces 7270 of resilient legs 7272 engage camming lockout surfaces 7212 of cartridge jaw 7202 while knife clip 7262 is still attached to knife sled 7236. Engagement between lockout cam surfaces 7270 and lockout surfaces 7212 of cartridge jaw 7202 laterally compress resilient legs 7272 toward each other, further cinching down on lockout engagement surfaces 7245 of knife sled 7236 (see FIG. 55B), thereby inhibiting knife 7206 from being fired distally.

Therefore, lockout assembly 7260 may allow firing of knife 7206 when a pre-fired cartridge 7210 is suitably loaded into cartridge jaw 7202 in accordance with the description herein. Additionally, lockout assembly 7260 is configured to inhibit firing of knife 7206 when an absent-cartridge condition or a spent-cartridge condition is present.

I. Ninth Illustrative End Effector with Lockout Assembly

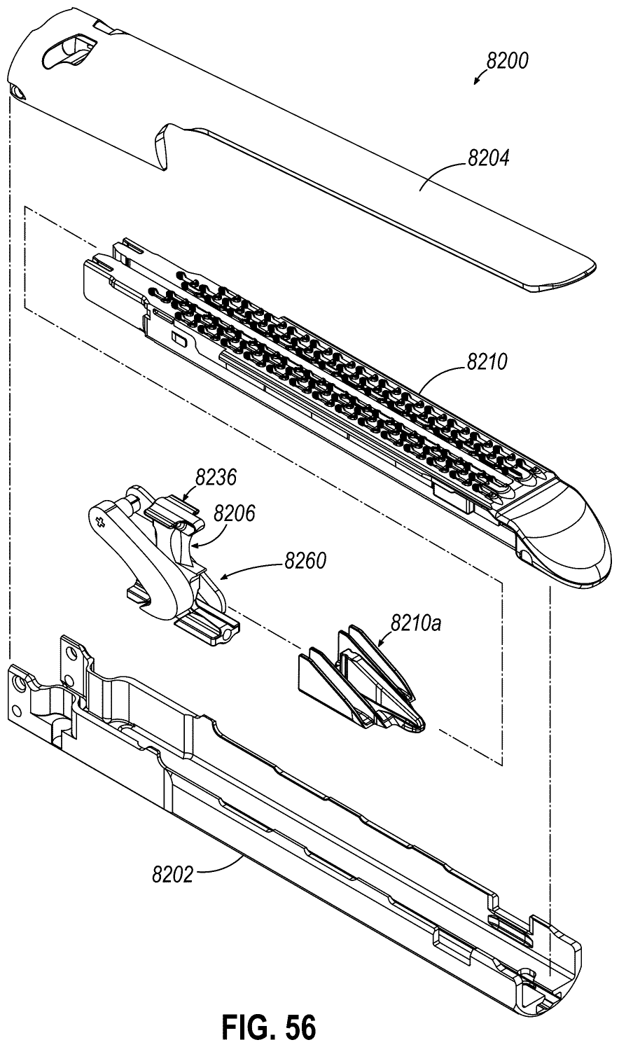

FIGS. 56-60B show an illustrative end effector 8200 that may be readily incorporated into surgical instrument 1000 in replacement of end effector 200 described above. Therefore, end effector 8200 may be substantially similar to end effector 200, 1200, 2200, 2300, 3200, 4200, 5200, 6200, 7200 described above, with differences elaborated herein. As will be described in greater detail below, end effector 8200 includes a lockout assembly 8260 configured to inhibit firing of end effector 8200 in the spent-cartridge condition and the absent-cartridge condition.

End effector 8200 includes a cartridge jaw 8202, an anvil jaw 8204, a staple cartridge 8210, a cartridge sled 8210A, a knife 8206, and knife sled 8236; which may be substantially similar to cartridge jaw 202, anvil jaw 204, staple cartridge 210, cartridge sled 210A, knife 206, and knife sled 236 described above, respectively, with differences elaborated herein. Therefore, cartridge jaw 8202 and anvil jaw 8204 are pivotally coupled to each other and may pivot relative to each other between an open position (similar to end effector 200 shown in FIG. 8A) and a clamping position (similar to end effector 200 shown in FIGS. 8C-8D), in response to movement of knife sled 8236 in accordance with the description herein.

Anvil jaw 8204 includes a longitudinally extending upper knife channel 8224 that may be substantially similar to longitudinally extending upper knife channel 224 described above. Similarly, cartridge jaw 8202 includes an elongated channel 8208 and a lower knife channel 8230, which may be substantially similar to elongate channel 208, and lower knife channel 230 described above, respectively, with differences elaborated below.

Knife sled 8236 includes a vertical column 8235, an upper knife tab 8238, and lower knife tab 8246; which may be substantially similar to vertical column 235, upper knife tab 238, and lower knife tab 246 described above, respectively, with differences elaborated below. Therefore, upper knife tab 8238 and lower knife tab 8246 may actuate within upper knife channel 8224 and lower knife channel 8230, respectively, as knife sled 8236 acutes relative to jaws 8202, 8204 in accordance with the description herein, thereby assisting in pivoting anvil jaw 8204 from the open position into the clamping position.

Staple cartridge 8210 slidably houses cartridge sled 8210A. Staple cartridge defines an elongated knife channel 8215 that is substantially similar to elongated knife channel 215 described above, with differences elaborated herein. Elongated channel 8208 of cartridge jaw 8202 may selectively receive a first staple cartridge 8210 such that an unfired staple cartridge 8210 may be loaded into cartridge jaw 8202 and fired in accordance with the description herein. Next, the spent staple cartridge 8210 may be removed from cartridge jaw 8202 such that a new, unfired staple cartridge staple cartridge 8210 may be loaded into cartridge jaw 8202.

As mentioned above, end effector 8200 includes a lockout assembly 8260 configured to inhibit firing of end effector 8200 in both the spent-cartridge condition and the absent-cartridge condition. In the current example, lockout assembly 8260 includes a hook 8262, a rocker 8264, and a lever 8266. Hook 8262 and rocker 8264 are each pivotally attached to distal retainer 324 such that hook 8262 and rocker 8264 may pivot about axis 8270 relative to distal retainer 324 and cartridge jaw 8202. Hook 8262 and rocker 8264 are suitably attached to each other such that pivotal movement of rocker 8264 about axis 8270 causes corresponding pivotal movement of hook 8262 about axis 8270, and vice versa.

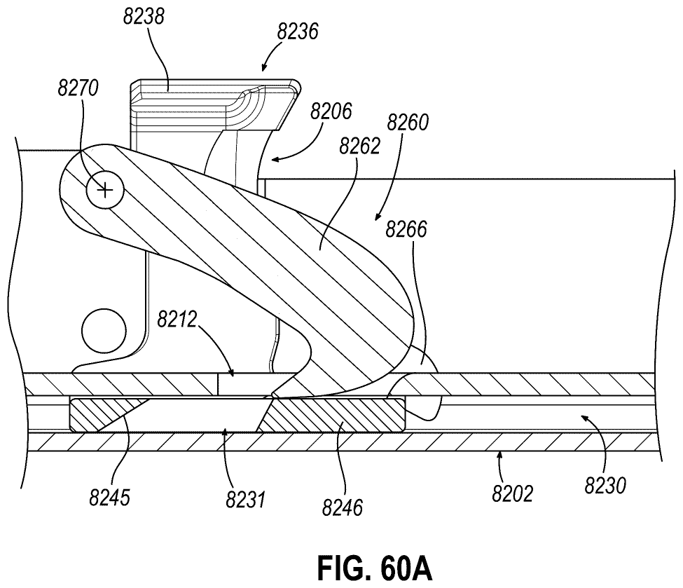

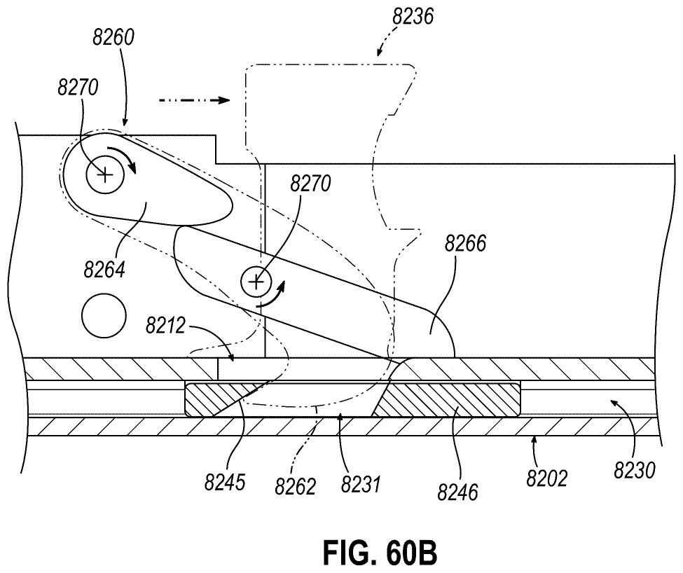

Cartridge jaw 8202 defines an opening 8212 adjacent to hook 8262 such that a distal end of hook 8262 may pivot into lower knife channel 8230. Further, lower knife tab 8246 of knife sled 8236 defines an opening 8231 that is dimensioned to receive a distal end of hook 8262 in the locked-out configuration (see FIG. 60B), thereby allowing hook 8262 to inhibit distal actuation of knife 8206 in accordance with the teachings herein. In particular, in the locked-out configuration, hook 8262 engages a lockout engagement surface 8245 of lower knife tab 8246, thereby inhibiting further distal actuation of knife 8206. Hook 8262 is biased toward the locked-out configuration. Hook 8262 may be biased toward the locked-out configuration using any suitable means as would be apparent to one skilled in the art in view of the teachings herein. For example, a torsion spring may be utilized to bias hook 8262 toward the locked-out configuration. As another example, the weight of hook 8262 may suitably bias hook 8262 toward the locked-out configuration.

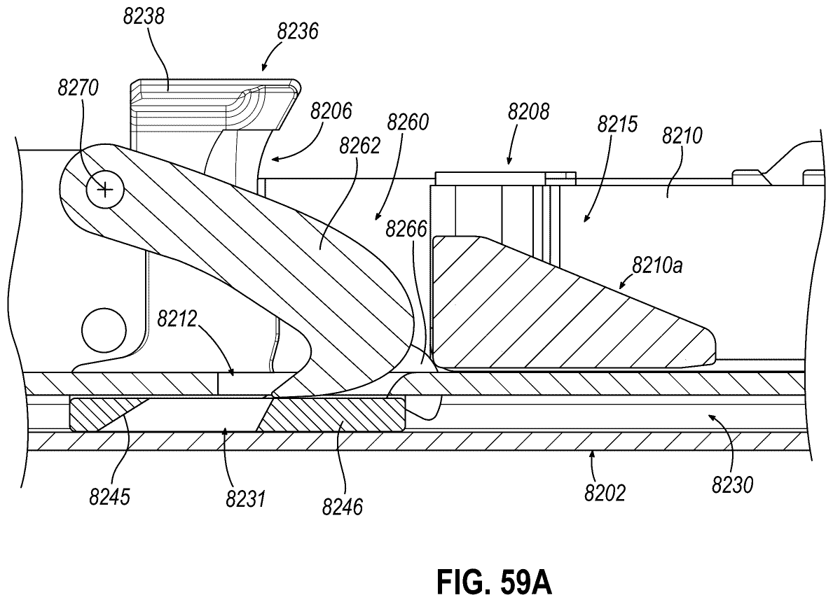

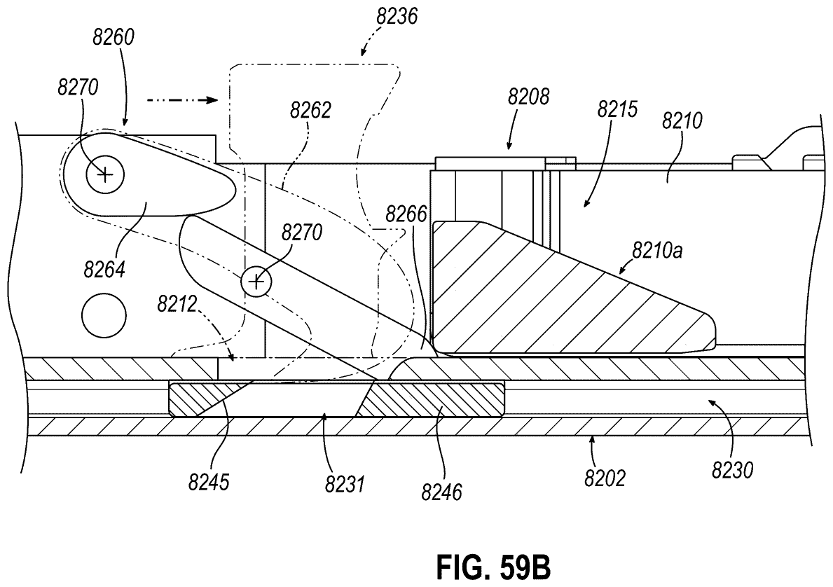

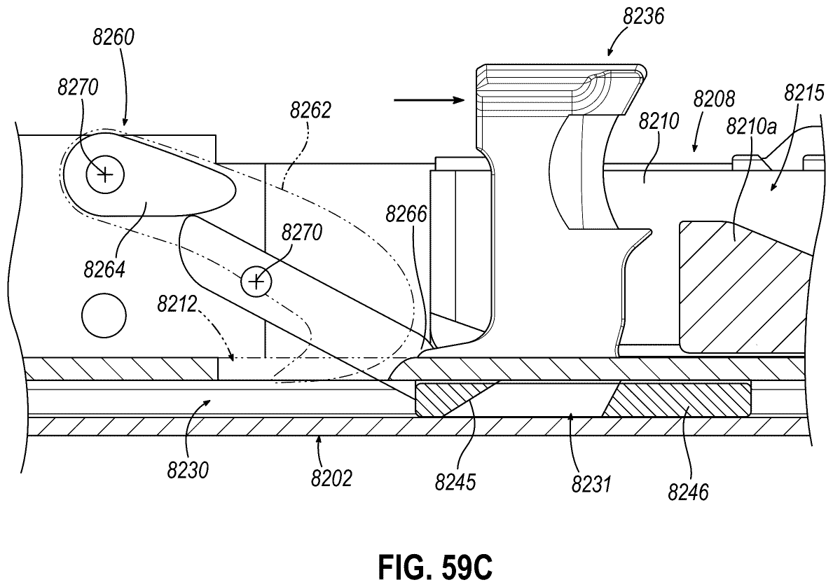

Lever 8266 is pivotally attached to knife sled 8236 such that lever 8266 actuates with knife sled 8236 and knife 8206 in accordance with the description herein, but also such that lever 8266 may pivot about axis 8272 relative to knife sled 8236. While knife 8206 is in and/or adjacent to the proximal position (see FIGS. 59A and 60A), a proximal end of lever 8266 is engaged with rocker 8264. Therefore, while knife 8206 is located at/near the proximal position (see FIGS. 59A and 60A), the pivotal position of lever 8266 about axis 8272 may dictate the pivotal position of rocker 8264 and hook 8262 via engagement between lever 8266 and rocker 8264.

Lever 8266 is suitably long enough such that when a pre-fired staple cartridge 8210 is loaded into cartridge jaw 8202 in accordance with the teachings herein, and knife 8206 is initially actuated distally from the proximal position (see FIG. 59A), a proximal end of lever 8266 suitably engages a pre-fired cartridge sled 8210 prior to hook 8262 engaging lockout engagement surface 8245 (see FIG. 59B). Engagement between the pre-fired cartridge sled 8210 and lever 8266 pivots lever 8266 about axis 8272, which in turn pivots rocker 8264 and hook 8262 upwards such that the distal end of hook 8262 may not engage lockout engagement surface 8245 of lower knife tab 8246. With hook 8262 pivoted upwards, knife 8206 may be fired distally in accordance with the teachings herein (see FIG. 59C). As knife 8206 is fired through jaws 8202, 8204 and cartridge 8210 in accordance with the description herein, lever 8266 is suitably dimensioned to fit within an elongated slot of cartridge 8210 such that lever 8266 does not inhibit distal actuation of knife 8206 during a suitable firing stroke in accordance with the description herein. It should be understood that when knife 8206 actuates proximally back into a pre-fired position after completing a firing stroke in accordance with the teachings herein, the proximal end of lever 8226 may suitably reengage the underside of rocker 8264.

If a spent-cartridge 8210 is loaded into cartridge jaw 8202 (i.e., a spent-cartridge condition), or a cartridge 8210 is absent from cartridge jaw 8202 (i.e., an absent-cartridge condition), when knife 8206 is initially actuated distally from the proximal position (see FIG. 60A), lever 8226 will refrain from pivoting rocker 8264 and hook 8262 upwards since lever 8226 does not engage a cartridge sled 8210. Therefore, the biased nature of hook 8262 drives hook 8254 into engagement with lockout engagement surface 8245 of lower knife tab 8246 (see FIG. 60B), thereby inhibiting further distal actuation of knife 8206 in accordance with the description herein.

### III. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus (1000) comprising: (a) an end effector (1200, 2200, 2300, 3200, 4200, 5200) operable to clamp, staple, and cut tissue, comprising: (i) a first jaw (1202, 2202, 2302, 3202, 4202, 5202) configured to selectively receive a staple cartridge (1210, 2210, 2310, 3210, 4210, 5210), (ii) a second jaw (1202), wherein the first jaw and the second jaw are configured to transition between an open position and a closed position, and (iii) a knife (1206, 2206, 2306, 3206, 4206, 5206) configured to actuate relative to the first jaw and the second jaw from a pre-fired position along a firing stroke while the staple cartridge is housed within the first jaw to thereby cut and staple tissue clamped by the first and second jaws; (b) a flexible coil (508, 1508, 510, 1510) operatively attached to the knife, wherein the flexible coil is configured to drive the knife distally from the pre-fired position along the firing stroke; and (c) a pivoting lock (1270, 2270, 2370, 3270, 4270, 5270) configured to pivot about a pivot axis relative to the knife between an unlocked configuration and a locked-out configuration, wherein the pivoting lock is configured to inhibit distal actuation of the knife in the locked-out configuration and permit distal translation of the knife in the unlocked configuration, wherein the pivoting lock is configured to assume the locked-out configuration in the absence of an unspent staple cartridge in the first jaw and the unlocked configuration in the presence of an unspent staple cartridge in the first jaw, wherein the knife is configured to actuate the pivoting lock relative to the first jaw and the second jaw along the firing stroke, wherein the pivot axis is vertically offset relative to the flexible coil.

### Example 2

The apparatus (1000) of Example 1, wherein the flexible coil comprises a first flexible coil (508, 1508) operatively attached to an upper portion (1238, 2238, 2338, 3238, 4238, 5238) of the knife (1206, 2206, 2306, 3206, 4206, 5206), wherein the apparatus further comprises a second flexible coil (510, 1510) operatively attached to a lower portion (1246, 2246, 2346, 3246, 4246, 5246) of the knife, wherein the first flexible coil and the second flexible coil are configured to cooperatively drive the knife (1206, 2206, 2306, 3206, 4206, 5206) distally, wherein the pivot axis is located vertically between the first flexible coil and the second flexible coil.

### Example 3

The apparatus (1000) of Example 2, wherein, while in the unlocked configuration, the pivoting lock (1270, 2270, 2370, 3270, 4270, 5270) is located, in its entirety, vertically between the first flexible coil (508, 1508) and the second flexible coil (510, 1510).

### Example 4

The apparatus (1000) of Example 2 or 3, wherein, while in the locked-out configuration, at least a portion of the pivoting lock (1270, 2270, 2370, 3270, 4270, 5270) is located distally relative to the first flexible coil (508, 1508) and the second flexible coil (510, 1510).

### Example 5

The apparatus (1000) of any one or more of Examples 2 through 4, wherein the first flexible coil (508, 1508) and the second flexible coil (510, 1510) engage the knife (1206, 2206, 2306, 3206, 4206, 5206) at a first location and a second location, respectively.

### Example 6

The apparatus (1000) of Example 5, wherein, while in the unlocked configuration, the pivoting lock (1270, 2270, 2370, 3270, 4270, 5270) is located, in its entirety, vertically between the first location and the second location.

### Example 7

The apparatus (1000) of any one or more of Examples 5 through 6, wherein, while in the locked-out configuration, at least a portion of the pivoting lock (1270, 2270, 2370, 3270, 4270, 5270) is located vertically outside the first location and the second location.

### Example 8

The apparatus (1000) of any one or more of Examples 1 through 7, wherein the pivoting lock (1270, 2270, 2370, 3270, 4270, 5270) is biased toward the locked-out configuration.

### Example 9

The apparatus (1000) of any one or more of Examples 1 through 8, wherein the pivoting lock (1270, 2270, 2370, 3270, 4270, 5270) is configured to enter the locked-out configuration when the knife (1206, 2206, 2306, 3206, 4206, 5206) is actuated distally from the pre-fired position without a staple cartridge (1210, 2210, 2310, 3210, 4210, 5210) in the first jaw (1202, 2202, 2302, 3202, 4202, 5202).

### Example 10

The apparatus (1000) of any one or more of Examples 1 through 9, wherein the pivoting lock (1270, 2270, 2370, 3270, 4270, 5270) is configured to enter the locked-out configuration when the knife (1206, 2206, 2306, 3206, 4206, 5206) is actuated distally from the pre-fired position with a spent staple cartridge (1210, 2210, 2310, 3210, 4210, 5210) in the first jaw (1202, 2202, 2302, 3202, 4202, 5202).

### Example 11

The apparatus (1000) of any one or more of Examples 1 through 10, wherein the firs jaw (1202, 2202, 2302, 3202, 4202, 5202) defines a recessed pocket (1212, 2212, 2312, 3212, 4212, 5212) configured to engage the pivoting lock (1270, 2270, 2370, 3270, 4270, 5270) in the locked-out configuration.

### Example 12

The apparatus (1000) of Example 11, wherein the end effector (1200, 2200, 2300, 3200, 4200, 5200) further comprises the staple cartridge (1210, 2210, 2310, 3210, 4210, 5210), wherein the staple cartridge comprises a sled (1210A, 2210A, 2310A, 3210A, 4210A, 5210A), wherein the sled, while in a pre-fired position, is interposed between the recessed pocket (1212, 2212, 2312, 3212, 4212, 5212) and the pivoting lock (1270, 2270, 2370, 3270, 4270, 5270).

### Example 13

The apparatus (1000) of any one or more of Examples 1 through 12, wherein the knife (1206, 2206, 2306, 3206, 4206, 5206) is configured to actuate the first jaw (1202, 2202, 2302, 3202, 4202, 5202) and the second jaw (1202) into the closed position in response to the knife actuating distally from the pre-fired position.

### Example 14

The apparatus (1000) of any one or more of Examples 1 through 13, further comprising a spring (1265, 2264, 2364, 3264, 4264, 5264) interposed between the pivoting lock (1270, 2270, 2370, 3270, 4270, 5270) and the knife (1206, 2206, 2306, 3206, 4206, 5206).

### Example 15

The apparatus (1000) of Example 13, wherein the spring (1265, 2264, 2364, 3264, 4264, 5264) comprises either a coil spring, a torsion spring, or a leaf spring.

The following clauses also relate to various non-exhaustive ways in which the teachings herein may be combined or applied.

Clause 1. An apparatus comprising:
(a) an end effector operable to clamp, staple, and cut tissue, comprising:
   (i) a first jaw configured to selectively receive a staple cartridge,
   (ii) a second jaw, wherein the first jaw and the second jaw are configured to transition between an open position and a closed position, and
   (iii) a knife configured to actuate relative to the first jaw and the second jaw from a pre-fired position along a firing stroke while the staple cartridge is housed within the first jaw to thereby cut and staple tissue clamped by the first and second jaws;
(b) a flexible coil operatively attached to the knife, wherein the flexible coil is configured to drive the knife distally from the pre-fired position along the firing stroke; and
(c) a pivoting lock configured to pivot about a pivot axis relative to the knife between an unlocked configuration and a locked-out configuration, wherein the pivoting lock is configured to inhibit distal actuation of the knife in the locked-out configuration and permit distal translation of the knife in the unlocked configuration, wherein the pivoting lock is configured to assume the locked-out configuration in the absence of an unspent staple cartridge in the first jaw and the unlocked configuration in the presence of an unspent staple cartridge in the first jaw, wherein the knife is configured to actuate the pivoting lock relative to the first jaw and the second jaw along the firing stroke, wherein the pivot axis is vertically offset relative to the flexible coil.

Clause 2. The apparatus of clause 1, wherein the flexible coil comprises a first flexible coil operatively attached to an upper portion of the knife, wherein the apparatus further comprises a second flexible coil operatively attached to a lower portion of the knife, wherein the first flexible coil and the second flexible coil are configured to cooperatively drive the knife distally, wherein the pivot axis is located vertically between the first flexible coil and the second flexible coil.

Clause 3. The apparatus of clause 2, wherein, while in the unlocked configuration, the pivoting lock is located, in its entirety, vertically between the first flexible coil and the second flexible coil.

Clause 4. The apparatus of clause 3, wherein, while in the locked-out configuration, at least a portion of the pivoting lock is located distally relative to the first flexible coil and the second flexible coil.

Clause 5. The apparatus of clause 4, wherein the first flexible coil and the second flexible coil engage the knife at a first location and a second location, respectively.

Clause 6. The apparatus of clause 5, wherein, while in the unlocked configuration, the pivoting lock is located, in its entirety, vertically between the first location and the second location.

Clause 7. The apparatus of clause 6, wherein, while in the locked-out configuration, at least a portion of the pivoting lock is located vertically outside the first location and the second location.

Clause 8. The apparatus of clause 1, wherein the pivoting lock is biased toward the locked-out configuration.

Clause 9. The apparatus of clause 1, wherein the pivoting lock is configured to enter the locked-out configuration when the knife is actuated distally from the pre-fired position without a staple cartridge in the first jaw.

Clause 10. The apparatus of clause 1, wherein the pivoting lock is configured to enter the locked-out configuration when the knife is actuated distally from the pre-fired position with a spent staple cartridge in the first jaw.

Clause 11. The apparatus clause 1, wherein the first jaw defines a recessed pocket configured to engage the pivoting lock in the locked-out configuration.

Clause 12. The apparatus of clause 11, wherein the end effector further comprises the staple cartridge, wherein the staple cartridge comprises a sled, wherein the sled, while in a pre-fired position, is interposed between the recessed pocket and the pivoting lock.

Clause 13. The apparatus of clause 1, wherein the knife is configured to actuate the first jaw and the second jaw into the closed position in response to the knife actuating distally from the pre-fired position.

Clause 14. The apparatus of clause 1, further comprising a spring interposed between the pivoting lock and the knife.

Clause 15. The apparatus of clause 14, wherein the spring comprises either a coil spring, a torsion spring, or a leaf spring.

Clause 16. An apparatus comprising:
(a) an end effector operable to clamp, staple, and cut tissue, comprising:
   (i) a first jaw configured to selectively receive a staple cartridge,
   (ii) a second jaw, wherein the first jaw and the second jaw are configured to transition between an open position and a closed position, and
   (iii) a knife configured to actuate relative to the first jaw and the second jaw from a pre-fired position along a firing stroke while the staple cartridge is housed within the first jaw to thereby cut and staple tissue clamped by the first and second jaws;
(b) a flexible coil operatively attached to an upper portion of the knife, wherein the flexible coil is configured to drive the knife distally from the pre-fired position along the firing stroke;
(c) a pivoting lock pivotally coupled to the knife about a pivot axis, wherein the pivoting lock is configured to inhibit distal actuation of the knife in a locked-out configuration and permit distal translation of the knife in an unlocked configuration, wherein the pivoting lock is configured to assume the locked-out configuration in the absence of an unspent staple cartridge in the first jaw and the unlocked configuration in the presence of an unspent staple cartridge in the first jaw, wherein the knife is configured to actuate the pivoting lock and the pivot axis relative to the first jaw and the second jaw along the firing stroke.

Clause 17. The apparatus of clause 16, wherein the knife comprises an upper tab and a lower tab, wherein the upper tab is attached to the flexible coil, wherein the apparatus further comprises a second flexible coil configured to cooperatively drive the knife distally in conjunction with the flexible coil, wherein the lower tab is attached to the second flexible coil.

Clause 18. The apparatus of clause 17, wherein the pivot axis is located between the upper tab and the lower tab.

Clause 19. The apparatus of clause 16, wherein the pivoting lock extends distally from the knife.

Clause 20. An apparatus, comprising
(a) an end effector operable to clamp, staple, and cut tissue, comprising:
   (i) a first jaw configured to selectively receive a staple cartridge,
   (ii) a second jaw pivotally attached to the second jaw, wherein the first jaw and the second jaw are configured to transition between an open position and a closed position, and
   (iii) a knife configured to actuate relative to the first jaw and the second jaw from a pre-fired position along a firing stroke while the staple cartridge is housed within the first jaw to thereby cut and staple tissue clamped by the first and second jaws, wherein the knife is configured to drive the first jaw and the second jaw toward the closed position;
(b) an articulation section configured to deflect the end effector within a first plane of articulation and a second plane of articulation;
(c) a flexible coil operatively attached to an upper portion of the knife, wherein the flexible coil is configured to drive the knife distally from the pre-fired position along the firing stroke;
and
(d) a pivoting lock pivotally coupled to the knife about a pivot axis, wherein the pivoting lock is configured to inhibit distal actuation of the knife in the locked-out configuration and permit distal translation of the knife in the unlocked configuration, wherein the pivoting lock is configured to assume the locked-out configuration in the absence of an unspent staple cartridge in the first jaw and the unlocked configuration in the presence of an unspent staple cartridge in the first jaw, wherein the knife is configured to actuate the pivoting lock and the pivot axis relative to the first jaw and the second jaw along the firing stroke.

### IV. Miscellaneous

It should be understood that any one or more of the teachings, expressions, versions, examples, etc. described herein may be combined with any one or more of the other teachings, expressions, versions, examples, etc. that are described herein. The above-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Furthermore, any one or more of the teachings herein may be combined with any one or more of the teachings disclosed in U.S. Pat. App. No. [Atty. Ref. END9622USNP2], entitled “Surgical Stapler with Firing Lockout Feature Coupled to End Effector Jaw,” filed on even date herewith; and/or U.S. Pat. App. No. [Atty. Ref. END9622USNP3], entitled “Surgical Stapler with Firing Lockout Feature Coupled to End Effector Retainer,” filed on even date herewith. The disclosure of each of the above patent references is incorporated by reference herein, in its entirety.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as those made available by Auris Health, Inc. of Redwood City, CA or by Intuitive Surgical, Inc., of Sunnyvale, California.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1.-15. (canceled)

16. An apparatus comprising:
(a) an end effector operable to clamp, staple, and cut tissue, comprising:
(i) a first jaw configured to selectively receive a staple cartridge,
(ii) a second jaw, wherein the first jaw and the second jaw are configured to transition between an open position and a closed position, and
(iii) a knife configured to actuate relative to the first jaw and the second jaw from a pre-fired position along a firing stroke while the staple cartridge is housed within the first jaw to thereby cut and staple tissue clamped by the first and second jaws, wherein the knife comprises a column and a tab;
(b) a flexible coil operatively attached to the knife, wherein the flexible coil is configured to drive the knife distally from the pre-fired position along the firing stroke; and
(c) a pivoting lock configured to pivot about a pivot axis relative to the knife between an unlocked configuration and a locked-out configuration, wherein the pivoting lock is configured to receive and transfer a firing force from the knife to the first jaw via engagement of the pivoting lock with the tab of the knife to thereby inhibit distal actuation of the knife in the locked-out configuration, wherein the pivoting lock is configured to permit distal translation of the knife in the unlocked configuration, wherein the pivoting lock is configured to assume the locked-out configuration in the absence of an unspent staple cartridge in the first jaw and the unlocked configuration in the presence of an unspent staple cartridge in the first jaw, wherein the knife is configured to actuate the pivoting lock relative to the first jaw and the second jaw along the firing stroke, wherein the pivot axis is vertically offset relative to the flexible coil.

17. The apparatus of claim 16, wherein the flexible coil comprises a first flexible coil operatively attached to an upper portion of the knife, wherein the apparatus further comprises a second flexible coil operatively attached to a lower portion of the knife, wherein the first flexible coil and the second flexible coil are configured to cooperatively drive the knife distally, wherein the pivot axis is located vertically between the first flexible coil and the second flexible coil.

18. The apparatus of claim 17, wherein, while in the unlocked configuration, the pivoting lock is located, in its entirety, vertically between the first flexible coil and the second flexible coil.

19. The apparatus of claim 18, wherein, while in the locked-out configuration, at least a portion of the pivoting lock is located distally relative to the first flexible coil and the second flexible coil.

20. The apparatus of claim 19, wherein the first flexible coil and the second flexible coil engage the knife at a first location and a second location, respectively.

21. The apparatus of claim 20, wherein, while in the unlocked configuration, the pivoting lock is located, in its entirety, vertically between the first location and the second location.

22. The apparatus of claim 21, wherein, while in the locked-out configuration, at least a portion of the pivoting lock is located vertically outside the first location and the second location.

23. The apparatus of claim 16, wherein the pivoting lock is biased toward the locked-out configuration.

24. The apparatus of claim 16, wherein the pivoting lock is configured to enter the locked-out configuration when the knife is actuated distally from the pre-fired position without a staple cartridge in the first jaw.

25. The apparatus of claim 16, wherein the pivoting lock is configured to enter the locked-out configuration when the knife is actuated distally from the pre-fired position with a spent staple cartridge in the first jaw.

26. The apparatus claim 16, wherein the first jaw defines a recessed pocket configured to engage the pivoting lock in the locked-out configuration.

27. The apparatus of claim 26, wherein the end effector further comprises the staple cartridge, wherein the staple cartridge comprises a sled, wherein the sled, while in a pre-fired position, is interposed between the recessed pocket and the pivoting lock.

28. The apparatus of claim 16, wherein the knife is configured to actuate the first jaw and the second jaw into the closed position in response to the knife actuating distally from the pre-fired position.

29. The apparatus of claim 16, further comprising a spring interposed between the pivoting lock and the knife.

30. The apparatus of claim 29, wherein the spring comprises either a coil spring, a torsion spring, or a leaf spring.

31. An apparatus comprising:
(a) an end effector operable to clamp, staple, and cut tissue, comprising:
(i) a first jaw configured to selectively receive a staple cartridge, wherein the first jaw defines a first knife channel,
(ii) a second jaw defining a second knife channel, wherein the first jaw and the second jaw are configured to transition between an open position and a closed position, and
(iii) a knife configured to actuate relative to the first jaw and the second jaw from a pre-fired position along a firing stroke while the staple cartridge is housed within the first jaw to thereby cut and staple tissue clamped by the first and second jaws, wherein the knife comprises a first tab and a second tab, wherein the first tab is slidably housed within the first knife channel of the first jaw along at least a portion of the firing stroke, wherein the second tab is slidably housed within the second knife channel of the second jaw along at least the portion of the firing stroke;
(b) a flexible coil operatively attached to an upper portion of the knife, wherein the flexible coil is configured to drive the knife distally from the pre-fired position along the firing stroke;
(c) a pivoting lock pivotally coupled to the knife about a pivot axis, wherein the pivoting lock is configured to inhibit distal actuation of the knife in a locked-out configuration and permit distal translation of the knife in an unlocked configuration, wherein the pivoting lock is configured to assume the locked-out configuration in the absence of an unspent staple cartridge in the first jaw and the unlocked configuration in the presence of an unspent staple cartridge in the first jaw, wherein the knife is configured to actuate the pivoting lock relative to the first jaw and the second jaw along the firing stroke,
wherein the first tab and the second tab are configured to rotate into contact with the first jaw and the second jaw, respectively, in response to the pivoting lock inhibiting distal actuation of the knife in the locked-out configuration.

32. The apparatus of claim 31, wherein the knife comprises an upper tab and a lower tab, wherein the upper tab is attached to the flexible coil, wherein the apparatus further comprises a second flexible coil configured to cooperatively drive the knife distally in conjunction with the flexible coil, wherein the lower tab is attached to the second flexible coil.

33. The apparatus of claim 32, wherein the pivot axis is located between the upper tab and the lower tab.

34. The apparatus of claim 31, wherein the pivoting lock extends distally from the knife.

35. An apparatus, comprising
(a) an end effector operable to clamp, staple, and cut tissue, comprising:
(i) a first jaw configured to selectively receive a staple cartridge,
(ii) a second jaw pivotally attached to the first jaw, wherein the first jaw and the second jaw are configured to transition between an open position and a closed position, and
(iii) a knife configured to actuate relative to the first jaw and the second jaw from a pre-fired position along a firing stroke while the staple cartridge is housed within the first jaw to thereby cut and staple tissue clamped by the first and second jaws, wherein the knife is configured to drive the first jaw and the second jaw toward the closed position, wherein the knife comprises a column having a side, wherein the side defines an angled cutout;
(b) an articulation section configured to deflect the end effector within a first plane of articulation and a second plane of articulation;
(c) a flexible coil operatively attached to an upper portion of the knife, wherein the flexible coil is configured to drive the knife distally from the pre-fired position along the firing stroke; and
(d) a pivoting lock comprising a proximal projection slidably contained within the angled cutout of the knife, wherein the pivoting lock is pivotally coupled to the knife about a pivot axis via the proximal projection and the angled cutout, wherein the pivoting lock is configured to inhibit distal actuation of the knife in the locked-out configuration and permit distal translation of the knife in the unlocked configuration, wherein the pivoting lock is configured to assume the locked-out configuration in the absence of an unspent staple cartridge in the first jaw and the unlocked configuration in the presence of an unspent staple cartridge in the first jaw, wherein the knife is configured to actuate the pivoting lock relative to the first jaw and the second jaw along the firing stroke.
